(19) United States
(12) Patent Application Publication
(10) Pub. No.: US 2019/0004052 A1
(43) Pub. Date: Jan. 3, 2019

(54) METHODS OF CHARACTERISING CANCER

(71) Applicants: Thomas Mole HERD, Eastleigh Hampshire (GB); Robert Scott GREENFIELD, Hartsdale, NY (US)

(72) Inventors: Thomas Mole HERD, Eastleigh Hampshire (GB); Robert Scott GREENFIELD, Hartsdale, NY (US)

(21) Appl. No.: 15/737,613

(22) PCT Filed: Jun. 20, 2016
(86) PCT No.: PCT/EP2016/064219

§ 371 Date: Dec. 18, 2017

Related U.S. Application Data
(60) Provisional application No. 62/181,429, filed on Jun. 18, 2015.

(51) Int. Cl. G01N 33/574; C07K 16/36

(52) U.S. Cl. CPC G01N 33/57488; G01N 2440/14; G01N 2333/7454; C07K 16/36

(57) ABSTRACT

The present invention concerns the use of immunoassays for the characterisation, diagnosis, screening and monitoring of cancer and other diseases. In particular said immunoassays involve the use of polyclonal and monoclonal antibodies against different regions in tissue factor, particularly urinary tissue factor, and methods for generating the same. Preferably said antibodies are specific for the tissue factor signal transduction peptide region (anti-TF-STP antibodies). In particular, the present invention relates to antibodies and the use thereof which are specific for phosphorylated isoforms of TF-STP. The invention further concerns use of these anti-TF-STP antibodies to quantitate TF-STP isoforms in biological fluids, in particular urine. Also described herein is the use of said antibodies in immunoassays for the characterisation, diagnosis, screening and monitoring of cancer and other diseases, specific polyclonal and monoclonal antibodies against different regions in tissue factor, and methods for generating the same.

### FIELD

The present invention concerns methods of quantifying Tissue factor (TF, CD142). The invention further concerns the use of TF in immunoassays for the characterisation, diagnosis, screening and monitoring of cancer and other diseases. Furthermore, the present invention discloses polyclonal and monoclonal antibodies against different regions in TF, particularly signal transduction peptide (STP) portion of TF and methods for generating the same. In particular, the present invention relates to antibodies which are specific for phosphorylated isoforms of TF-STP, and methods of detecting and quantifying said phosphorylated TF. The invention further concerns use of these anti-TF-STP antibodies to quantitate TF-STP in biological fluids, in particular urine.

### BACKGROUND

### Description of the Related Art

A series of coordinated reactions takes place in the body whenever blood clots. The major physiological initiator of these reactions is a membrane-bound glycoprotein known as Tissue Factor (TF), which is normally separated from the blood stream by the vascular endothelium. Bleeding, caused by injury or tissue damage, activates a complex enzyme cascade as TF becomes exposed to the bloodstream. This activates the extrinsic pathway of the coagulation system. TF binds to and activates Factor VII to FVIIa leading to activation of Factor X to Xa and ultimately the activation of Prothrombin to thrombin (see FIG. 1).

However, in disease states, leucocytes or the vascular endothelium may abnormally express TF to cause intravascular coagulation. The blood coagulation cascade is also relevant to diseases such as haemophilia in which sufferers are deficient in blood proteins necessary for normal clotting, and is linked to vascular diseases such as heart attacks and strokes in which clotting may lead to occlusion of blood vessels.

Coagulation is also activated in inflammatory illnesses via induction of TF by inflammatory cells. Cancer is also associated with hyper thrombotic states due to up-regulation of TF in the cancer cells themselves. Tissue Factor plays in these processes and also the highly significant part it plays in angiogenesis, cancer, metastasis, inflammation, drug resistance, fertility and wound healing.

TF Immunoassays

TF has been measured in urine and other body fluids using immunoassays such as ELISA formats (Fareed et al, 1995). Immunoassays utilize antibodies against the tissue factor protein to measure TF antigen or protein levels rather than the biological activity of TF. TF ELISAs in the past have used antibodies directed against the extracellular domain (e.g. Imubind Tissue Factor ELISA (REF 845) Sekisui Diagnostics, Stamford, Conn.). The extracellular domain is mainly responsible for the procoagulant activity of TF. Such ELISAs measure various forms of TF that contain the intact extracellular domain which may not be adequate as suggested above for the KCA activity assays. The presence of proteolytic fragments of TF in a sample would interfere with the measurement of full length TF (i.e. urinary TF) because the proteolytic fragments would block the binding of antibodies to the full length antibody. Other forms of TF (i.e. alternately spliced TFs) which also have a native extracellular domain would also interfere with the measurement of TF for similar reasons.

Immunoassays directed against the extracellular portion of TF are not suitable for specifically detecting and quantitating different modifications of the signal transduction peptide (STP) portion of TF. Such modifications include phosphorylation at Ser253 and Ser258. Thus, to accurately measure the various TF-STP isoforms, for example the TF-STP in urine (uTF-STP), there is a requirement for a different approach to the design of the immunoassay. More specifically it requires a novel set of antibodies that will distinguish the TF-STP isoforms in a two site immunoassay format.

The potential for other uses of immunoassays against TF-STP (for example uTF-STP) are also possible. According to scientific evidence, the uTF and hence the uTF-STP isoforms in the urine are derived from kidney tubular cells. According to theory, it is believed that kidney tubular cells are acted upon by various biological mediators (e.g. cytokines, chemokines, lymphokines, TNF, etc) and are induced to express TF on their cell surface. The sloughing off of the induced membrane-bound TF into microparticles or the destruction of the kidney tubular cells via apoptosis or other cytocidal mechanisms cause TF of various forms (phosphorylated and unphosphorylated) to appear in the urine. Thus, elevation of TF-STP in the urine may be an indicator of the presence of disease due to the sloughing into the urine of TF-STP isoforms made in the kidney cells. Therefore TF-STP, and particularly uTF-STP, may be a biological marker for cancer and other disease states. Furthermore, the quantitation of TF-STP, in particular in urine (uTF-STP), may be useful in the diagnosis and detection of cancer and other disease conditions. In addition to cancer, treatment of such medical conditions as kidney failure, diabetes, inflammation, autoimmune diseases, and sepsis may benefit from knowing the TF-STP isoform levels. This also suggests that drugs targeted against the TF-STP may be useful for treating such diseases.

Koyama et al [1994] (British J Haematology 87:343-347) reported quantitation of TF in blood and urine from normal and patients with cancer and other diseases using an ELISA directed against the extracellular portion of TF. Koyama observed that the TF in biological fluids was present in the expected molecular weight range of 45 kD but was also found to be degraded or present in a high molecular weight aggregated form. They reported low levels of TF in urine of healthy patients but did not report on urinary TF levels in cancer or other disease states. However, Koyama reported that TF levels in plasma of healthy patients (149 pg/ml) was not significantly different from cancer patients with various lymphomas and leukemias (115-174 pg/ml). Thus, an ELISA directed at the extracellular domain of TF was not suitable for quantitation of TF for diagnosing cancer based upon elevation of TF levels in biological fluids.

Monoclonal antibodies have been made against amino acids (aa) 254-263 in the c-terminal region of TF (Carson and Yoder, Blood Coagul Fibrinolysis (1992) 3: 779-787). They utilized the monoclonal antibodies to show that the c-terminal peptide can be proteolytically cleaved from TF, to characterize the distribution of vesicles containing TF and to determine the orientation of TF within the vesicles. There are reports describing the construction and use of single-site immunoassays against TF-STP or the phosphorylated isoforms of the TF-STP to investigate biochemical mechanisms involving TF, however, there are no reports suggesting that quantitating TF-STP and TF-STP isoforms are useful for the purpose of diagnosing, screening or monitoring cancer.

### SUMMARY

It is thus an aim of the present invention to provide methods and kits for characterising diseases by determining the quantities of the different isoforms of the intracellular domain of tissue factor.

In a first embodiment therefore, the present invention provides a method quantifying one or more tissue factor signal transduction peptide (TF-STP) isoforms in a test sample, preferably in a urine sample.

In a further embodiment, the present invention provides a method for characterising a cancer or other disease comprising the steps of first quantifying one or more tissue factor signal transduction peptide (TF-STP) isoforms in a test sample obtained from a subject and, secondly, comparing the quantity of the one or more TF-STP isoforms or the ratio of the quantities of two or more TF-STP isoforms in the test sample to the quantity of those TF-STP isoforms or the ratio thereof in a normal or healthy control sample, wherein a quantity of the one or more TF-STP isoform in the test sample or ratio thereof which falls outside of a predetermined normal range is indicative of a characteristic of a disease. The predetermined normal range in this embodiment may be the same as, different to, and/or or overlapping with the predetermined normal range for that isoform in another embodiment.

Methods according to the present invention are also used in screening a population for disease. For example, the method may be applied to samples obtained from every member of a population. Additionally, results from each sample may be collated and analysed statistically.

Methods according to the present invention may characterise various cancer types or potentially other diseases such as kidney disease, hyperthyroidism, glomerulonephritis, or diabetes. In particular said method may characterise solid cancers of the breast, ovary, colon, central nervous system, kidney, prostate, bladder, colorectal, liver, lung (non-small cell and small cell), brain, pancreas, stomach, oesophagus, head, or neck cancer type, or Hodgkins lymphoma, non-Hodgkins lymphoma, chronic lymphocytic leukemia, acute lymphocytic leukemia, chronic myeloid leukemia, or acute myeloid leukemia, or dialysis-dependent chronic kidney disease, nondialysis dependent chronic kidney disease, glomerulonephritis, nephropathy, nephritis, or renal failure.

In one embodiment of the invention the one or more TF-STP isoform is selected from the group consisting of phosphorylated TF-STP and unphosphorylated TF-STP, preferably wherein the phosphorylated TF-STP isoform is selected from TF-STP-Phosphoserine253 (TF-STP-Pser253, or when found in urine uTF-STP-Pser253) and TF-STP-Phosphoserine258 (TF-STP-Pser258 or when found in urine uTF-STP-Pser258) and TF-STP-Phosphoserine253,258 (or when found in urine, uTF-STP-Phosphoserine253,258).

In some embodiments, the test sample obtained from the subject is selected from the group consisting of fractionated or unfractionated urine, saliva, whole blood, plasma, platelet rich plasma (PRP), platelet poor plasma (PPP), and pooled normal plasma (PNP), preferably wherein the test sample is fractionated or unfractionated urine.

In another embodiment, two or more TF-STP isoforms are quantified by a method according to the invention. The ratio of the two or more TF-STP isoforms may then be calculated and compared to the ratio of those isoforms in a normal or healthy control sample, wherein a ratio of the two or more TF-STP isoforms in the test sample which falls outside of a predetermined normal range is indicative of a characteristic of a disease. The predetermined normal range for that ratio in this embodiment may be the same as, different to, and/or or overlapping with the predetermined normal range in another embodiment. Such characteristics of disease may include the nature of the disease, the presence of the disease, the stage of the disease, the grade of the disease, or another characteristic.

In a further, related embodiment, two or more of the quantities and/or ratios may be measured. In this embodiment, two or more of the quantities and/or ratios falling outside of a predetermined normal range is indicative of a characteristic of a disease. The predetermined normal range for those quantities and/or ratios in this embodiment may be the same as, different to, and/or or overlapping with the predetermined normal range in another embodiment. Such characteristics of disease may include the nature of the disease, the presence of the disease, the stage of the disease, the grade of the disease, or another characteristic.

In embodiments wherein the disease is a cancer, the characteristic of the cancer is the cancer type, the presence of the cancer, the stage of an associated tumor, and/or the grade of an associated tumor. For example, the cancer type may be breast, bladder, colon, prostate, or lung cancer, or a leukaemia, the stage of the tumor may be stage 0, I, II, Ill, or IV, and the grade of the tumor may be grade G1, G2, or G3.

Another embodiment envisaged is a method according to the present invention for in vitro diagnosing a disease wherein at least one step is performed outside of the human or animal body. For example, each step of an ELISA according to the present invention, carried out on a sample from a human subject is performed in vitro outside the human body. Diseases include cancer, kidney disease, hyperthyroidism, glomerulonephritis, or diabetes. In particular a breast, ovary, colon, central nervous system, kidney, prostate, bladder, colorectal, liver, lung (non-small cell and small cell), brain, pancreas, stomach, oesophagus, head, or neck cancer or Hodgkins lymphoma, non-Hodgkins lymphoma, chronic lymphocytic leukemia, acute lymphocytic leukemia, chronic myeloid leukemia, or acute myeloid leukemia, or dialysis-dependent chronic kidney disease, nondialysis dependent chronic kidney disease, glomerulonephritis, nephropathy, nephritis, or renal failure.

In another embodiment, a method according to the present invention may be repeated at least once on samples obtained from the same subject at defined intervals. It is envisaged that this practise may, for example, allow for the progression of a disease to be monitored by detecting the stage of the disease at each interval, or, for the effectiveness of a treatment to be monitored. It is envisaged that this practise may apply to any disease according to the invention as herein described.

In another embodiment, the test sample is obtained from a subject previously diagnosed with the disease being characterised. It is envisaged that this practise may, for example, identify whether the disease is still present in the subject or not. For example, it may be detected whether or not a treatment has successfully caused a cancer to enter remission.

In a further embodiment of the present invention, the one or more TF-STP isoforms (such as uTF-STP isoforms) in a test sample is quantified by non-immunological methods such as mass spectroscopy, HPLC, gas chromatography, or by immunological methods employing one or more antibodies that specifically bind to phosphorylated or unphosphorylated TF-STP isoforms. Suitable immunological methods include immunoassays, for example a radioimmunoassay or an Enzyme-Linked Immunosorbent Assay (ELISA), such as an antibody-sandwich ELISA. Antibodies suitable for use in embodiments employing an immunological quantification method include antibodies, antibody conjugates, or antigen-binding fragments thereof which specifically bind an unphosphorylated or phosphorylated TF-STP isoform, preferably wherein the antibodies, antibody conjugates, or antigen-binding fragments thereof are specific for any of phosphorylated TF-STP, TF-STP-Pser253 or TF-STP-Pser258 (uTF-STP-Pser253 or uTF-STP-Pser258).

In another embodiment of the invention, a kit is provided comprising at least a first antibody, antibody conjugate, or antigen binding fragment thereof specific for phosphorylated or unphosphorylated uTF-STP isoform, preferably wherein the at least first antibody, antibody conjugate, or antigen binding fragment thereof is specific for one of unphosphorylated TF-STP, TF-STP-Pser253 or TF-STP-Pser258, and a ligand that binds to the antibody, antibody conjugate, or antigen binding fragment thereof, wherein the ligand comprises a member with reporter activity, preferably wherein the member with reporter activity is Horse Radish Peroxidase.

In a further embodiment of the invention, a kit is provided comprising a second antibody, antibody conjugate, or antigen-binding fragment thereof, wherein the second antibody, antibody conjugate, or antigen-binding fragment thereof is specific for a different epitope on the same TF-STP isoform for which the first antibody, antibody conjugate, or antigen-binding fragment thereof is specific.

A further embodiment of the invention provides a kit wherein the ligand binds to the conjugated moiety of the antibody conjugate, for example wherein the conjugated moiety comprises biotin or poly-biotin and the ligand comprises streptavidin.

In another embodiment, a kit is provided wherein the ligand comprises a label. Said label may for example comprise anti-immunoglobulin antibody, an anti-immunoglobulin peptide, protein A, protein G, and Protein L.

In some embodiments, the kit includes a microtiter plate comprising immobilised anti-TF-STP antibody, preferably specific for one of unphosphorylated TF-STP, TF-STP-Pser253 or TF-STP-Pser258.

Also herein are described specific antibodies, preferably comprising SEQ ID NOs: 11 and/or 12.

Other embodiments and advantages of the invention will be apparent in part in the description, examples, and figures, which follow, and in part, may be obvious from this description, examples, and figures, or may be learned from the practice of the invention.

### BRIEF DESCRIPTION OF THE FIGURES

FIG. 1: A pictorial representation of Cellular tissue factor initiated coagulation, illustrating he role of Tissue Factor (TF) and TF-bearing cells.

FIG. 2: A pictorial representation of the mature 263 amino acid sequence of human TF (see also SEQ ID NO:10). The extracellular, membrane (or transmembrane) and C-terminal cytoplasmic (or intracellular) domains are illustrated thereon. This figure excludes the 32 amino acid signal sequence which is cleaved.

FIG. 3: A pictorial model of TF showing a representation of the three domains in situ in the cell.

FIG. 4: Pictorial illustration of a two site immunoassays against the TF-STP.

FIG. 5: Signal transduction pathways involving tissue factor

FIG. 6a: Anti-TF1 screening data showing the binding affinity of the supernatant sample towards each antigen when probed with goat-anti-mouse-HRP as a secondary antibody (Biorad, Catalogue number:—170-6516) at 1:5000 dilution in P.B.S. Sample was added at 1:100 dilution in P.B.S.

FIG. 6b: Anti-TF2 screening data showing the binding affinity of the supernatant sample towards each antigen when probed with goat-anti-mouse-HRP as a secondary antibody (Biorad, Catalogue number:—170-6516) at 1:5000 dilution in P.B.S. Serum was added at 1:100 dilution in P.B.S.

FIG. 6c: Specificity of anti-TF-Pser²⁵⁸ MAb which is of the IgG isotype

FIG. 6d: Specificity of clones producing anti-TF-Pser253 MAbs which are of the IgM isotype

FIG. 7: Optical density measurements indicating the specificity of binding of hybrid monoclonal anti-TF-Pser²⁵³ antibody to TF2-Pser²⁵³ peptide and lack of binding to unphosphorylated TF3 or TF1-Pser²⁵⁸ peptide.

FIG. 8: ELISA plates were coated with anti-TF1 Pab and anti-TF3 Pab-biotinylated was used as detection antibody. TF3 synthetic peptide CRKAGVGQSWKENSPLNVS comprising the last entire c-terminal region of TF was used as the antigen

FIG. 9: ELISA plates were coated with anti-TF2 MAb and anti-TF3 Pab-biotinylated was used as detection antibody in the two site ELISA. 9A: Peptide TF3 CRKAGVGQSWKENSPLNVS was used as the antigen standard. 9B: Full length (aa1-263) recombinant TF was used as the antigen standard.

FIG. 10a: Effect of different buffer solutions on readings from ELISA using anti-TF2 MAb as capture antibody and anti-TF3 Pab-biotin as the detection antibody.

FIG. 10b: Effect of buffer conditions on ELISA using recombinant TF as antigen

FIG. 11: Specificity of ELISA Format for unphosphorylated TF-STP

FIG. 12a: ELISA standard curve for detection of TF-Pser258 antigen

FIG. 12b: The ELISA was specific for TF3-Pser258 as there was no detection of unphosphorylated TF3 peptide nor unphosphorylated rTF at 200 ug/ml levels of each antigen.

FIG. 13: Elisa specific for quantitation of TF-Pser253.

FIG. 14: Quantities of a) unphosphorylated uTF, b) uTF-STP-Pser²⁵⁸, and c) uTF-STP-Pser²⁵³ measured by ELISA according to the present invention in urine samples from patients with bladder, breast, colon, or prostate cancer, or healthy individuals.

FIG. 15: Dot plot summarizing results of quantitation of uTF in fractionated urine samples from 28 normal, 25 colon cancer, 60 prostate cancer, and 5 bladder cancer subjects by ELISA.

FIG. 15a. Bar chart showing results of uTF-STP-Pser258 quantitation by ELISA of unfractionated urine samples. Levels of uTF-STP-Pser258 are significantly higher (P<0.001) in bladder cancer compared to healthy, colon cancer, breast cancer and prostate cancer.

FIG. 15b. ROC curve showing 86% sensitivity and 91% specificity of the uTF-STP-Pser258 biomarker in ELISA of unfractionated bladder cancer and healthy urine samples. FIG. 15c. Bar chart showing results of uTF-STP-Pser258 quantitation by ELISA of fractionated urine samples. Levels of uTF-STP-Pser258 are significantly higher (P<0.001) in bladder cancer compared to healthy, colon cancer, breast cancer and prostate cancer.

FIG. 15d. ROC curve showing 89% sensitivity and 88% specificity of the uTF-STP-Pser258 biomarker in ELISA of fractionated bladder cancer and healthy urine samples in the detection of bladder cancer.

FIG. 16a. Bar chart showing results of unphosphorylated uTF-STP (TF3) quantitation by ELISA of unfractionated urine samples. Levels of unphosphorylated uTF-STP are significantly higher (P<0.001) in bladder cancer compared to healthy, colon cancer, breast cancer and prostate cancer.

FIG. 16b. ROC curve showing 76% sensitivity and 77% specificity of the unphosphorylated uTF-STP (TF3) biomarker in ELISA of unfractionated bladder cancer and healthy urine samples in the detection of bladder cancer.

FIG. 16c. Bar chart showing results of unphosphorylated uTF-STP (TF3) quantitation by ELISA of fractionated urine samples. Levels of uTF-STP are significantly higher (P<0.001) in bladder cancer compared to healthy, colon cancer, breast cancer and prostate cancer.

FIG. 16d. ROC curve showing 76% sensitivity and 77% specificity of the unphosphorylated uTF-STP (TF3) biomarker in ELISA of fractionated bladder cancer and healthy urine samples in detecting bladder cancer

FIG. 17a. Bar chart showing results of uTF-STP-Pser253 quantitation by ELISA of unfractionated urine samples. Levels of uTF-STP-Pser253 are significantly higher in colon cancer compared to bladder and breast cancer (P<0.001), healthy individuals (P<0.01), and prostate cancer (P<0.05).

FIG. 17b. ROC curve showing 71% sensitivity and 69% specificity of the uTF-STP-Pser253 biomarker in ELISA of unfractionated colon cancer and healthy urine samples in detection of colon cancer

FIG. 18a. Bar chart showing ratios of unphosphorylated uTF-STP in fractionated and unfractionated urine, determined by ELISA, from healthy individuals and patients with colon cancer, breast cancer, prostate cancer and bladder cancer.

FIG. 18b. ROC curve showing 75% sensitivity and 73% specificity of the unfractionated/fractionated uTF-STP (TF3) ratio in prostate cancer and healthy urine samples in detection of prostate cancer.

FIG. 18c. ROC curve showing 70% sensitivity and 64% specificity of the unfractionated/fractionated uTF-STP (TF3) ratio in breast cancer and healthy urine samples in detection of breast cancer.

FIG. 19a. Bar chart showing ratios of uTF-STP-Pser253 in fractionated and unfractionated urine, detected by ELISA, from healthy individuals and patients with colon cancer, breast cancer, prostate cancer and bladder cancer.

FIG. 19b. ROC curve showing 59% sensitivity and 51% specificity of the unfractionated/fractionated uTF-STP-Pser253 ratio in colon cancer and healthy urine samples in detection of colon cancer.

FIG. 20a. Bar chart showing ratios of uTF-STP (TF3) in fractionated and uTF-STP-Pser258 in unfractionated urine, detected by ELISA, from healthy individuals and patients with colon cancer, breast cancer, prostate cancer, and bladder cancer. Ratios are significantly lower in samples from breast (P<0.05) and prostate (P<0.001) cancers than from healthy subjects.

FIG. 20b. ROC curve showing 67% sensitivity and 69% specificity of the ratio of uTF-STP (TF3) in fractionated and uTF-STP-Pser258 unfractionated urine in breast cancer and healthy samples in detection of breast cancer.

FIG. 20c. ROC curve showing 72% sensitivity and 71% specificity of the ratio of uTF-STP (TF3) in fractionated and uTF-STP-Pser258 unfractionated urine in prostate cancer and healthy samples in detection of prostate cancer.

FIG. 21. Bar chart showing that the levels of uTF-STP-Pser258 in unfractionated and fractionated urine are correlated with stage of tumors from bladder, breast, colon, and prostate cancers.

FIG. 22. Bar chart showing the levels of unphosphorylated uTF-STP in unfractionated urine are correlated with stage of bladder cancer.

FIG. 23a. Bar chart showing that the levels of unphosphorylated uTF-STP in unfractionated and fractionated urine are correlated with grade of cancers.

FIG. 23b. Bar chart showing that the levels of unphosphorylated uTF-STP in unfractionated urine are correlated with grade of bladder cancers.

FIG. 24. Bar chart showing results of algorithm for screening population to preferentially identify colon, prostate and bladder cancers in a population

FIG. 25. Bar chart showing results of algorithm for screening population to preferentially identify colon, prostate and bladder cancers in a population

FIG. 26. Bar chart showing results of algorithm for screening population to preferentially identify bladder cancers in a population

FIG. 27. Bar chart showing results of algorithm for screening population to preferentially identify colon cancers in a population

FIG. 28. Bar chart showing results of algorithm for screening population to preferentially identify breast and prostate cancers in a population

SEQUENCES

SEQ ID NO: 1: CKENSPLNVS
SEQ ID NO: 2: CRKAGVGQSW
SEQ ID NO: 3: CRKAGVGQSWKENSPLNVS
SEQ ID NO: 4: CKEN(pS)PLNVS
SEQ ID NO: 5: CRKAGVGQ(pS)W
SEQ ID NO: 6: CRKAGVGQ-(pS)WKENSPLNVS
SEQ ID NO: 7: CRKAGVGQSWKEN(pS)PLNVS
SEQ ID NO: 8: CRKAGVGQ(pS)WKEN(pS)PLNVS
SEQ ID NO: 9:
METPAWPRVPRPETAVARTLLLGWVFAQVAGASGTTNTVAAYNLTWKSTN
FKTILEWEPKPVNQVYTVQISTKSGDWKSKCFYTTDTECDLTDEIVKDVK
QTYLARVFSYPAGNVESTGSAGEPLYENSPEFTPYLETNLGQPTIQSFEQ
VGTKVNVTVEDERTLVRRNNTFLSLRDVFGKDLIYTLYYWKSSSSGKKTA
KTNTNEFLIDVDKGENYCFSVQAVIPSRTVNRKSTDSPVECMGQEKGEFR
EIFYIIGAVVFVVIILVIILAISLHKCRKAGVGQSWKENSPLNVS
SEQ ID NO: 10:
SGTTNTVAAYNLTWKSTNFKTILEWEPKPVNQVYTVQISTKSGDWKSKCF
YTTDTECDLTDEIVKDVKQTYLARVFSYPAGNVESTGSAGEPLYENSPEF
TPYLETNLGQPTIQSFEQVGTKVNVTVEDERTLVRRNNTFLSLRDVFGKD
LIYTLYYWKSSSSGKKTAKTNTNEFLIDVDKGENYCFSVQAVIPSRTVNR
KSTDSPVECMGQEKGEFREIFYIIGAVVFVVIILVIILAISLHKCRKAGV
GQSWKENSPLNVS
SEQ ID NO: 11:
MRVLILLWLFTAFPGILSDVQLQESGPGLVKPSQSLSLTCTVTGYSITSD
YAWNWIRQFPGNKLEWMGYISYSGSTSYNPSLKSRISITRDTSKNQFFLQ
LNSVTTEDTATYYCARIRGYLAMDYWGQGTSVTVSSESQSFPNVFPLV
SEQ ID NO: 12:
MRSPAQFLGLLVLWIPGSTADIVMTQAAFSNPVTLGTSASISCRSSKSLL
HSNGITYLYWYLQKPGQSPQLLIYQMSNLASGVPDRFSSSGSGTDFTLRI
SRVEAEDVGVYYCAQNLELPPTFGGGTKLEIKRADAAPTVSIFPPSSEQL
TSGGASVVCFLNNFYPK
SEQ ID NO: 13: KENSPLNVS
SEQ ID NO: 14: KEN(pS)PLNVS

SEQ ID NO:1 (also referred to herein as TF1) shows the amino acids 255-263 of mature (lacking 33 amino acid signal sequence) human TF, with an additional cysteine at the N-terminal.

SEQ ID NO:2 (also referred to herein as TF2) shows the amino acids 245-254 of mature (lacking 33 amino acid signal sequence) human TF.

SEQ ID NO:3 (also referred to herein as TF3) shows the amino acids 245-263 of mature human TF.

SEQ ID NO:4 (also referred to herein as TF1-Pser258) shows the amino acids 255-263 of mature human TF, having a phosphorylated serine at position 258 and an additional cysteine at the N-terminal.

SEQ ID NO:5 (also referred to as TF2-Pser253) shows the amino acids 245-254 of mature human TF, having a phosphorylated serine at position 253.

SEQ ID NO:6 (also referred to herein as TF3-Pser253) shows the amino acids 245-263 of mature human TF, having a phosphorylated serine at position 253.

SEQ ID NO:7 (also referred to herein as TF3-Pser258) shows the amino acids 245-263 of mature human TF, having a phosphorylated serine at position 258.

SEQ ID NO:8 (also referred to herein as TF3-Pser253/258) shows the amino acids 245-263 of mature human TF, having two phosphorylated serine residues, at positions 253 and 258.

SEQ ID NO:9 shows the full length amino acid sequence of human Tissue Factor (human TF), including a 33 amino acid signal sequence.

SEQ ID NO:10 shows the amino acid sequence of mature human TF, lacking the 33 amino acid signal sequence.

Unless stated otherwise, amino acid numbering of any TF amino acid sequence referred to herein is given with reference to the position numbering of amino acids found in SEQ ID NO:10.

SEQ ID NO:11 shows the amino acid sequence of the heavy chain variable region of the antibody specific for SEQ ID NO:5, wherein SEQ ID NO:5 has a phosphorylated serine at position 253.

SEQ ID NO:12 shows the amino acid sequence of the light chain variable region of the antibody specific for SEQ ID NO:5, wherein SEQ ID NO:5 has a phosphorylated serine at position 253.

SEQ ID NO:13 shows the wild-type SEQ ID NO:1, i.e. amino acids 255-263 of mature (lacking 33 amino acid signal sequence) human TF, without an additional cysteine at the N-terminal.

SEQ ID NO:14 shows the wild-type SEQ ID NO:4, i.e. amino acids 255-263 of mature (lacking 33 amino acid signal sequence) human TF, having a phosphorylated serine at position 258, without an additional cysteine at the N-terminal.

### DETAILED DESCRIPTION

Description of the Related Art

Forms of Tissue Factor

Two forms of Tissue Factor (TF) have been described: free TF (which includes a soluble, alternatively spliced and bloodborne TF) and membrane-bound TF.

Soluble TF results from proteolytic cleavage at or near the linkage between the transmembrane and the extracellular domains of the TF molecule, forming protein fragments, whereas the other variants of free TF results from alternative splicing of the primary RNA transcript (Bogdanov et al.,2003; Bogdanov et al.,2006; Guo et al., 2001). These free forms of TF circulate in the plasma and are biologically active. Plasma TF can also be found circulating in association with cell-derived membrane microparticles. TF-bearing microparticles arise mainly from monocyte-macrophage membrane-lipid rafts or from regions of high raft content; in this case TF is called bloodborne. The circulating TF microparticles alone do not seem to effectively initiate coagulation, but when they bind and fuse with activated platelets via the mechanism involving P-selectin glycoprotein ligand-1 on the microparticles and P-selectin on the platelets, they initiate coagulation (del Conde, Shrimpton, Thiagarajan & Lopez, 2005).

The membrane-bound forms of TF include both cellular TF (such as that found on monocytes, macrophages, endothelial and tumor cells) and lipid-vesicle-bound TF in urine (such as in microparticles, exosomes) or in semen (prostasomes). On the plasma membrane of cells, TF resides mostly in a cryptic configuration. De-encryption occurs by breaking disulfide bond between two cysteine residues in the TF extracellular domain.

Expression of TF

TF is a constituent of both the subendothelial layer of the vascular wall and the extravascular tissue. It thereby forms a protective lining around the blood vessels and is ready to activate blood coagulation if vascular integrity is compromised (Ryan et al., 1992). Endothelial cells and blood monocytes (in contact with the bloodstream) do not constitutively express TF and do not have stores of TF. High levels of TF are expressed on many solid cancers and this correlates with a more aggressive and less treatable phenotype.

Although TF is not normally expressed by cells within the bloodstream, gene transcription and subsequent protein expression can be induced in monocytes, macrophages and endothelial cells by thrombin, endogenous inflammatory mediators such as interleukins 1 & 6, tumor-necrosis factor-alpha (TNF-α), vascular permeability factor (VPF), complement C5a, phorbol esters, plasma lipoproteins, plasma protein, collagen, immune complexes and microorganisms as well as other physiological and pathological mediators (Amirkhosravi et al., 1996; Østerud, Olsen & Wilsgard, 1990; Roth, 1994).

Platelets and lymphocytes can induce de novo TF biosynthesis in monocytes and endothelial cells. Likewise monocytes and natural killer cells can upregulate TF expression in endothelial cells (Napoleone, Di Santo & Lorenzet, 1997). This cell-cell cooperation is essential for TF induction in inflammatory disease and in the cell mediated immune response. Shearing forces and hypoxia also induce TF expression in many cells and provoke coagulation. TF activity also relies on changes in cellular phospholipid composition).

TF-Mediated Intracellular Signaling

Many studies collectively indicate that TF has many additional biological functions other than its most well known role as an essential molecular partner of FVIIa of the extrinsic pathway of the coagulation system.

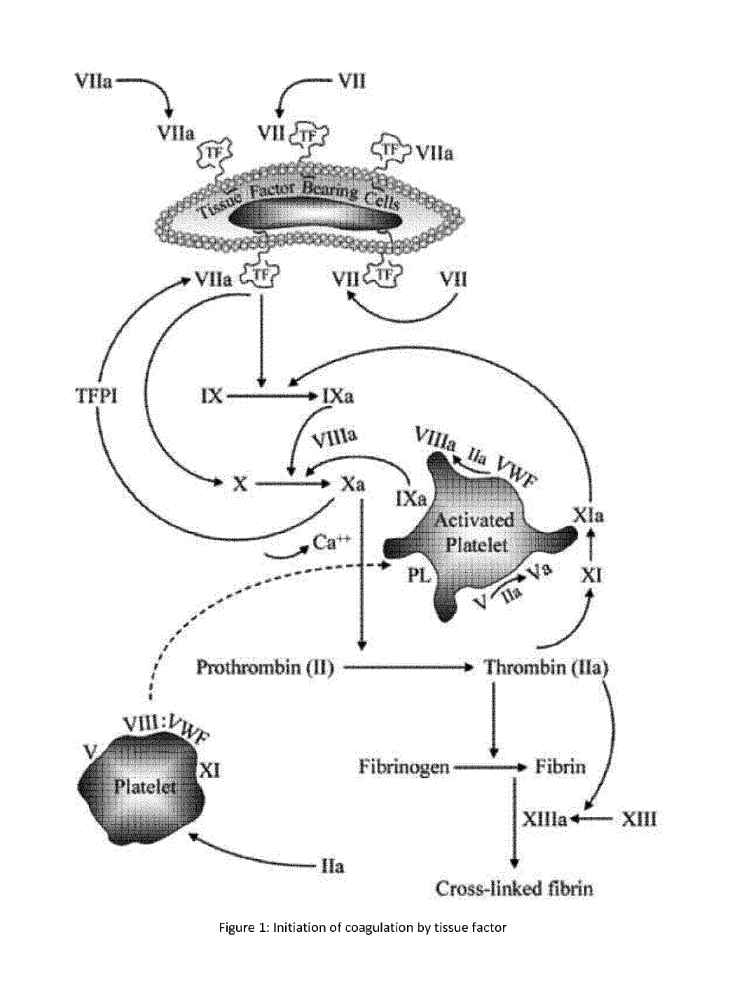

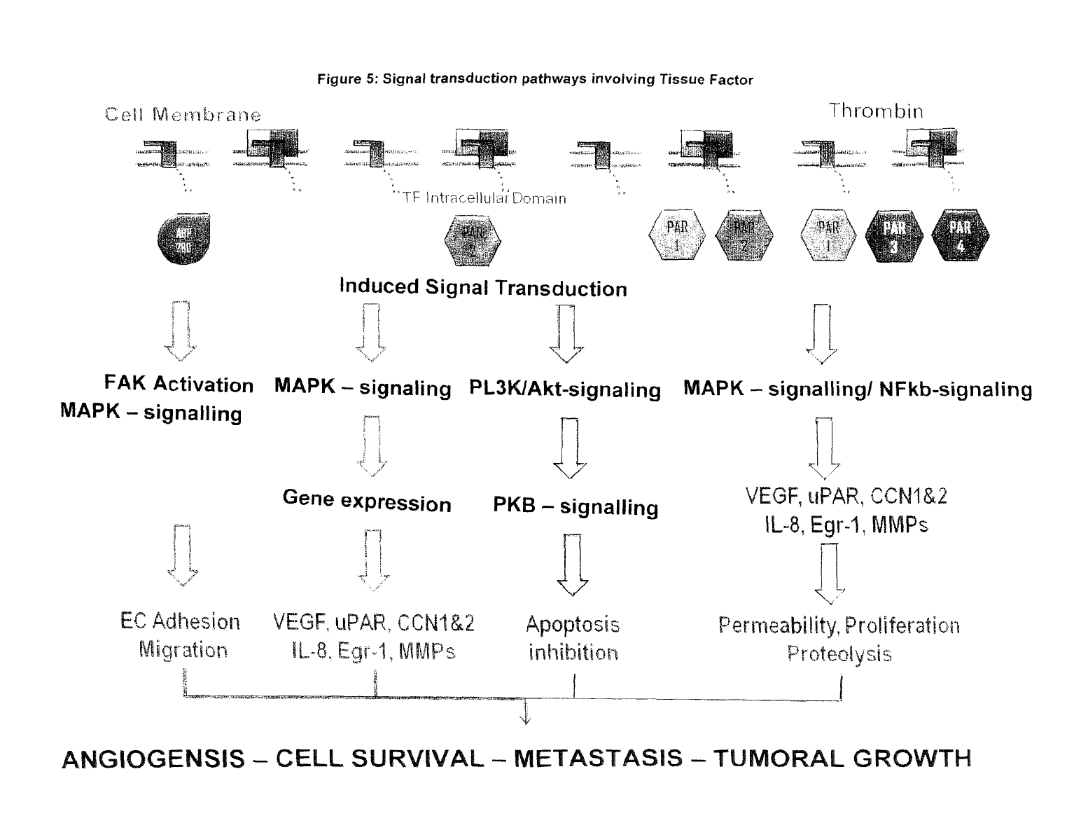

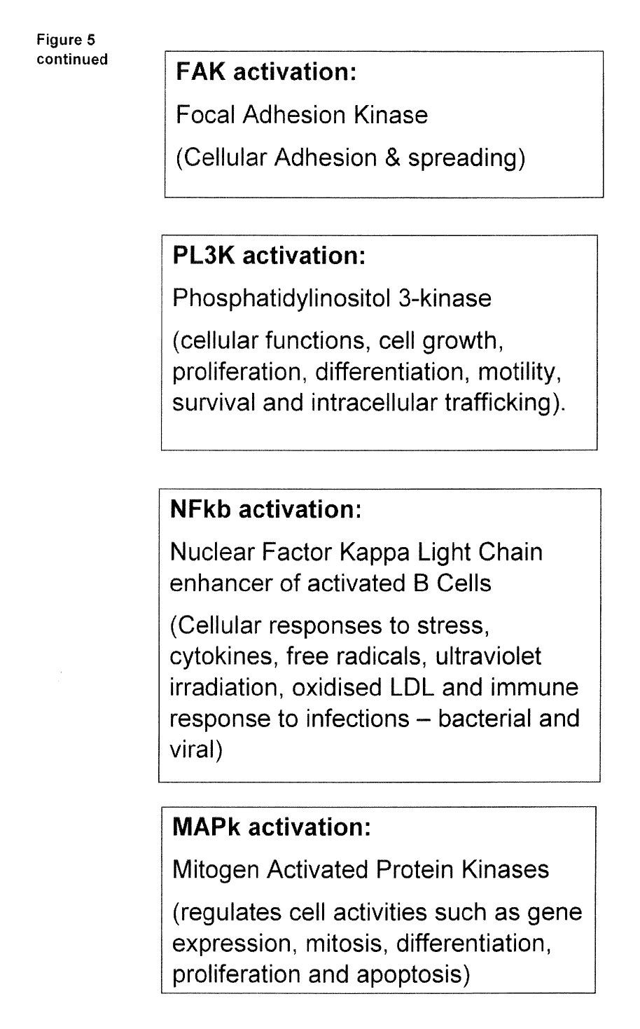

TF binding to FVIIa (as illustrated by FIG. 1) has important coagulation-independent functions, especially in angiogenesis, leukocyte migration, inflammation and the progression of lethal Escherichia coli sepsis. A variety of signal transduction events are elicited in TF-expressing cells upon addition of FVIIa. FVIIa complexation with the extracellular domain of TF thus not only stimulates haemostasis but also alters cellular physiology of the TF-expressing cell. The formation of TF/fVIIa complex at the surface of cells induces intracellular signal transduction pathways such as the activation of MAPK, NF-kB, FAK and PL3 kinase pathways (see FIG. 5) which are mediated by the phosphorylation and dephosphorylation of Ser 253 and Ser 258 in the intracellular STP region of TF.

These signal transduction pathways ultimately influence the gene transcription and expression of multiple proteins such as uPAR, VEGF, IL-8 and MMPs.

PL3 kinase activation constitutes an important branching point for further signal transduction routes, mainly related to anti-apoptotic processes, cell growth, differentiation and motility. A schematic representation of TF-VIIa protease-induced signaling is shown in FIG. 1. TF-VIIa activates PAR2 and the ternary complex of TF-VIIa-Xa activates both PAR1 and PAR2. Activation of PAR2-specific signaling pathway may lead to phosphorylation of TF cytoplasmic tail. ABP-280 plays a role in TF-signaling through its high affinity interaction with the TF cytoplasmic domain. In addition, TF-dependant generation of coagulation proteases activates multiple pathways involved in coagulation and signaling. The overall activation of this multiple cell signaling is responsible for the induction of angiogenesis, tumor growth and metastasis.

Several biochemical and biological studies clearly demonstrate the important role that tissue factor plays in cancer. TF itself is a regulator of many genes which contributes to carcinogenesis and to many biological processes that influence the progression and metastasis of cancer. Additionally, many biological factors affect the up-regulation of TF on normal as well as the tumor cells themselves, which also provides impetus for development and the promotion of cancers.

Tissue factor in the urine: Urinary Tissue Factor (uTF)—Background

Urine is known to possess powerful ‘procoagulant activity’ [PCA] that normalizes the clotting time of haemophiliac patients in vivo (von Kaula et al, 1954). von Kaulla noted that preparations obtained by adsorbing human urine with barium sulphate were largely associated with materials that normalized the clotting of haemophiliac plasma in vitro (von Kaula et al, 1965). It was demonstrated that minute amounts of these preparations had the ability to correct the clotting of patients who have a high titre of circulating ‘antithromboplastin’, now known as tissue factor pathway inhibitor [TFPI](von Kaula et a1,1963).

The PC activity of urine was first thought to be due to a tissue thromboplastin-related substance. It was later found that urine with PC activity catalysed the conversion of prothrombin to thrombin only in the presence of platelet factor 3, factor V and Ca²⁺—a prothrombinase type of activity, and therefore the substance causing this activity was classified as a ‘platelet cofactor’ rather than TF (Matsumura et al, 1970, Caldwell et a1,1963). Aoki and von Kaulla further investigated the interaction between platelet factor 3, factor V, prothrombin, and urinary PCA. They concluded that platelet factor 3 could be replaced by phospholipids in the activation of prothrombin with partially purified urinary procoagulant (uPC), provided that the lipid was used under optimal conditions. A similar observation was made by Joist and Alkiaersig, who noted that phospholipid or platelets merely enhanced urinary PC activity in the coagulation of plasma (Joist et al, 1967).

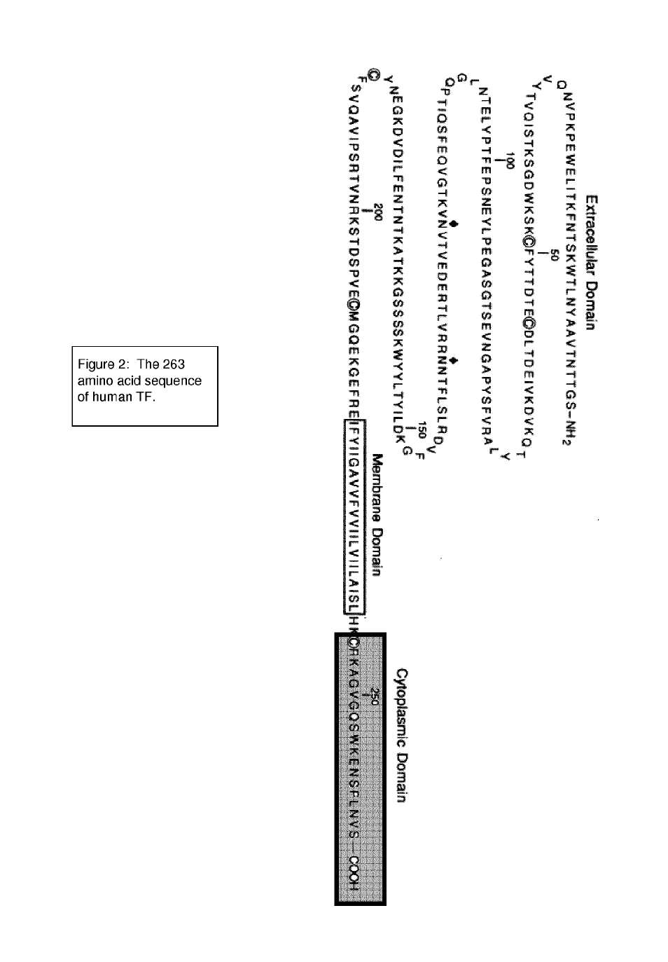

Subsequently, a factor that had the properties of TF was described in urine. Kurosawa et al. purified the uPC by chromatography on phenyl-sepharose and showed that it promoted clot formation in a factor VII-dependent manner (Kurosawa et al, 1984). Aoki and von Kaulla suggested that the uPC existed as small particles and they reported a factor with the properties of ‘prothrombinase’. Subsequently, it was demonstrated that uPC existed in lipid-associated vesicles and was mainly factor VII-dependent (Wiggins et al, 1987). The uPC can activate factor X in the presence of factor VII and calcium and was inhibited by concanavalin A (Zacharaski et al, 1974). Activity was restored by the addition of á-methyl-glucoside (Kurosawa et al, 1984). Later, it was confirmed that the procoagulant was TF by demonstrating almost total inhibition of the activity in the presence of a specific antibody to human TF (Carty et al, 1990). The activity appeared to be associated with membrane vesicles that passed through a 220 nM filter, but not one with 100 nM pores. The substance identified has been named urinary tissue factor (uTF) (see FIG. 2).

Following purification and the removal of contaminant Tamm-Horsfall mucoprotein, the procoagulant was found to exist in micro-aggregates. These micro-aggregates were composed of basic functional units of 151 kD consisting of two sub-units of 68 and 76 kD held together by disulphide bonds (Kurosawa et al, 1984). This contrasts with an established molecular weight for human TF of 43 kD. These differences probably reflect the relative impurity of the former preparation, since the mature TF mRNA transcript is the same in a variety of human tissues, including kidney. Indeed, a subsequent estimate of uTF using western blotting revealed that uTF has a similar molecular weight to TF derived from other human tissues [43 kD](Carty et al, 1990). The vesicles can be sedimented by ultracentrifugation (Carty 1990) and TF activity can be recovered by solubilizing the TF-containing pellet in the detergent â-octyl-glucopyranoside (Lwaleed et al, 1999). The vesicles seemed to be intimately associated with fibrin strands when the latter were generated in vitro (Carty 1990) and showed specific binding to gold-labelled murine anti-TF (Lwaleed et al, 1998).

Structure of TF

TF found in the urine is similar in structure to all full length membrane-bound glycosylated tissue factors. The amino acid sequence of the human tissue factor protein has been deduced from the isolation of the TF gene (CD142). The tissue factor protein is produced as a single chain protein consisting of 295 amino acids. The first 32 amino acids are cleaved off after synthesis yielding a final protein of 263 amino acids (see FIG. 2 and SEQ ID NO:10). The TF protein is glycosylated at several sites and has a nominal molecular weight of 43 to 45 kD.

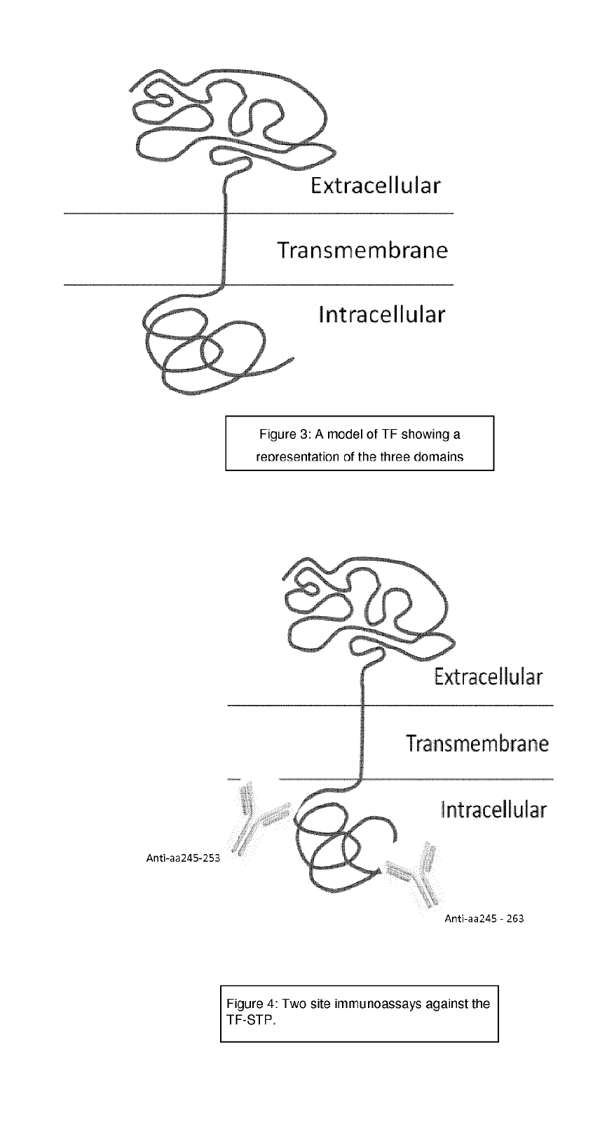

Human TF has three domains: 1) an extracellular domain; 2) a transmembrane domain; and 3) a C-terminal cytoplasmic intracellular domain (see FIGS. 3 and 4).

Each domain provides a critical function essential for the biology of TF. The extracellular domain (aa 1-219) is responsible for binding to various proteins in the coagulation system such as Factor VII, TFPI and Factor X. The transmembrane domain (aa 220-244) is highly hydrophobic and anchors TF in the plasma membrane of cells. The intracellular domain (aa245-263), which is also known as the signal transduction peptide (“STP”, as referred to herein) mediates signal transduction activity which leads to the activation and inactivation of various genes. The signal transduction function of tissue is vitally important in carcinogenesis.

Tissue factor found in human urine (urinary TF or uTF) is structurally and chemically identical to human tissue factor found elsewhere in the body. The “u” in term “uTF” only indicates the location in which the TF was found. Wherein the term “uTF” is used herein, it refers to TF found in the urine. Unless otherwise specified, as used here the terms “TF” and “uTF” can be used interchangeable. Antibodies specific for TF, a phosphorylated TF or a part thereof are also specific for uTF, a phosphorylated uTF or a part there of respectively, and vice versa.

Lwaleed et al, (1998) indicated that tissue factor found in the urine is not blood-derived. uTF is produced by the kidney itself (Bukovsky et al, 1992, Lwaleed et al, 1998; Matsumura and von Kaulla, 1968; Matsumura et al, 1968; Matsuda et al, 1979; Lwaleed et al, 2007; Lwaleed et al, 1999). Thus TF in the urine (also referred to herein as urinary TF and uTF) is the same TF protein molecules as described in the blood and other cells in the body.

Altered Forms of Tissue Factor

Two other TF forms have been described. Soluble TF results from proteolytic cleavage of TF at or near the linkage between the transmembrane and the extracellular domains of the TF molecule, forming a soluble protein fragment consisting entirely of the extracellular domain. Another soluble form of TF called alternatively spliced TF (asTF) does not have the transmembrane region nor the STP region and consequently is not anchored on cell membranes and thus plays no part in cell signaling.

Mechanism of TF Expression

Although TF is not normally expressed by cells within the bloodstream, gene transcription and subsequent protein expression can be induced in other cells. For example, TF expression can be induced in monocytes, macrophages, kidney tubular cells and endothelial cells by thrombin, endogenous inflammatory mediators such as interleukins (IL-1 & IL-6), tumor-necrosis factor-alpha (TNF-α), vascular permeability factor (VPF), complement C5a, phorbol esters, plasma lipoproteins, plasma protein, collagen, immune complexes and microorganisms as well as other physiological and pathological mediators (Amirkhosravi et al., 1996; Østerud, Olsen & Wilsgard, 1990; Roth, 1994).

TF Expression in Urine of Cancer Patients

Elevated TF levels and activity is found in the urine of patients with multiple cancer types. We speculate that the following mechanisms may explain the association of high levels of TF and TF activity in the urine with the presence of tumors that are far removed from the kidney.

The growth of a tumor in the body is a unique biological situation. The developing tumor is viewed as a foreign invader by the body. As such, the body attempts to mount a vigorous rejection of the growing tumor via activation of complex anti-inflammatory and immunological processes. A wide variety of inflammatory and immune cells become focused at the site of the growing tumor. These defensive cell networks that interact with the tumor produce soluble molecular mediators that provide communication links between the various inflammatory and immune cell networks. Among these soluble mediators are the tumor necrosis factor (TNF) family of molecular modulators. These TNF-related molecular mediators include TNF-α (Tissue Necrosis Factor-α), RANTES (Regulated-upon-Activation Normal T-cell Expressed and Secreted), TWEAK (member of TNF family), TRAIL (TNF-Related Apoptosis Including Ligand), FAS ligand (TNF Superfamily) among others. Each one of these soluble mediators plays a key role in inflammation, activation of cellular genes, proliferation, cell killing and apoptosis. (Sanchez-Nino et. al.)

One can speculate the following chain of events that occurs during tumor growth. As the tumor develops and grows in the prostate, colon or breast, the inflammatory and immune mechanisms are activated and come into play at the site of the tumor. During these ongoing defensive processes, TNF and/or TNF-like soluble mediators are generated at the site of the tumor. These soluble mediators enter the blood and/lymphatic system surrounding the tumors and are distributed throughout the body.

As the soluble cytokine mediator (e.g. TNF) in the blood passes through the kidney, it is concentrated within the glomerular network in the kidney. The soluble cytokine mediator such as TNF comes in contact with receptors on kidney tubular cells. TNF can be directly toxic to kidney cells and subsequently causes the death of kidney cells via ischemia and apoptosis. Apoptosis can result in sloughing of kidney tubular cell-derived TF-microvesicles sloughing off into the urine.

The soluble mediators can also activate other signal transduction pathways and causes the activation of the TF gene and ultimately increasing the expression of the TF protein on the surface of kidney tubular cells. For example, TNF-α generated at the site of the tumor mass travels to the kidney via the blood and/or lymph system and binds to specific receptors on the surface of kidney tubular cells. The binding of TNF to cell surface receptors activates NF-kappa B signal transduction pathways which leads to the activation of the TF gene and subsequent expression of the tissue factor protein on the membrane of the kidney tubular cells. This would be similar to the effect of LPS induced TF expression in kidney tubular cells observed by Lwaleed. Induction of excess TF in cells has been postulated to be toxic to the cell. To protect the kidney tubular cells from cell death induced by overexpression of TF, the kidney tubular cells slough off TF from their membrane in lipid microvesicles containing TF. The TF-bound microvesicles are deposited in the urine.

The close relationship between growing tumors, presence of soluble cytokines/biological response modifiers and TF expression in kidney cells may provide the mechanism by which TF is found in urine of patients with different cancer types. TNF-α and its family of related chemokines up-regulate tissue factor expression in many different cell types including kidney tubular cells. It also been shown that TNF is a major participant in the pathogenesis of kidney injury. TNF can cause a reduction of blood flow in the kidney, promote ischemia and cause the death of kidney tubular cells. TNF-α and its family of related chemokines in the TNF family are the key soluble mediators of inflammation that are up-regulated in tumor masses and released into blood circulation. According to these scenarios, any distant tumor type be that bladder, breast, colon, prostate or lung that can generate systemic TNF or TNF-like molecules can cause the appearance of TF in the urine of cancer patients. It may also be possible that different tumor types induce different soluble cytokine mediators that ultimately lead to the induction of TF within kidney cells. This is a proposed reasonable rationale which explains the biology of uTF.

As noted above, uTF has the same molecular structure as TF found in the blood and on the surface of tumor cells and monocytes, This also suggests that the signal transduction peptide (STP—see FIG. 2) which is also part of the uTF molecule can play a role in the cancer process by mediating signal transduction and regulation of gene expression within kidney cells. Since the STP region of TF plays a critical role in gene regulation mechanisms associated with TF, the STP region of TF should be a prime biomarker target for the development of cancer diagnostic assays, and in particular the STP region of uTF.

Phosphorylation of TF

The two faces of TF signaling involve crucial interactions of coagulation factor X or integrin (hypothetical) with an exosite in the TF extracellular domain. Signaling of the ternary TF-VIIa-Xa complex and the TF-VIIa binary complex may elicit distinct biological effects. Specific roles of ternary complex signaling in tumor biology have not been established, but this complex leads to thrombin generation and associated signaling. TF-VIIa signaling in association with integrins appears to be suited to regulate cell migration which in reverse can be controlled by the phosphorylation status of the TF cytoplasmic domain.

Signal Transduction and subsequent biological effects of TF are mediated through the phosphorylation and de-phosphorylation of two serine residues, which are numbered 253 and 258 with reference to the position numbering of the mature polypeptide shown in FIG. 2 and SEQ ID NO:10, in the intracellular cytoplasmic STP portion of TF (see FIG. 2).

The role of phosphorylation of the TF STP has been most intently studied in cancer cells. The importance of the phosphorylation of TF-STP in breast cancer was demonstrated by Ryden et al. (International Journal of Cancer 126:2330-2340, 2010) who showed by immunohistochemistry that phosphorylated TF (pTF) predicts poor prognosis in breast cancer. The recurrence-free survival (RFS) in the whole cohort was ˜90% during a median follow-up time of 50 months. All patients with recurrences had tumors expressing pTF (p=0.03) and PAR-2 (p=0.2). Furthermore, the co-expression of pTF and PAR-2 was found in all evaluable tumors from recurring patients, whereas none of the patients experiencing a recurrence had a tumor negative for co expression of pTF and PAR-2. The results support a role for PAR-2-induced TF phosphorylation in the prognosis of human breast cancer.

The current research suggests that in cancer cells phosphorylation of the two serine residues within the TF-STP at Ser253 and Ser258 act as on/off switches facilitating and preventing the release of TF into cell-derived microparticles respectively. The over accumulation of TF within the cancer cell appears to lead to cell death via the induction of apoptotic mechanisms. It is believed that larger/heavier TF-bearing microvesicles are released into biological fluids due to apoptosis. The phosphorylation of Ser253 acts to induce the incorporation of TF into small lipid vesicles (microparticles) through a mechanism called exocytosis that involves the cytoskeleton. This represents a molecular mechanism to explain the release of TF into microparticles by cancer cells and the positive correlation of TF with tumor aggressiveness and hyper-thrombotic state of cancer patients. In a complementary fashion, it has been postulated that the phosphorylation of Ser253 also induces mechanisms that lead to the phosphorylation of Ser258 which in turn causes dephosphorylation of Ser253 through phosphatase activation. Consequently the phosphorylation of Ser258 results in the activation of mechanisms of de-phosphorylation of Ser253 and terminates the release of TF-microparticles from the cancer cell surface [Collier and Ettelaie, J. Biol. Chem. (2011) 286:11977-11984; Dorfleutner and Ruf, Blood (2003) 102:3998-4005)].

As postulated in cancer cells (see above), similar phosphorylation mechanisms may occur for TF in the kidney. Thus, phosphorylation of Ser253 on TF in the membrane of kidney tubular would lead to shedding of TF-bearing microparticles cells into urine. Similarly, phosphorylation of Ser258 may result in dephosphorylation of Ser253 and suppression of the shedding of TF-microparticles into urine. The subsequent build-up of TF in the kidney tubular cells may lead to apoptosis and deposition of TF-containing cell debris into the urine.

Measurement of uTF in Various Diseases and Cancer Using Activity Assays

Advances in the understanding of the molecular structure and the biochemistry of TF have led to the development of assays for measuring various structural components of TF. Different methods have been used to assess TF at the cellular and plasmatic levels, and in other body fluids, with substantial clinical potential. Most of the studies of TF in urine (uTF) have employed assays that rely on the measurement of the extracellular domain which is the site of procoagulant activity. These activity assays also only measure fully functional uTF and focus on the extracellular domain of uTF. One-stage and two-stage kinetic chromogenic assays (KCA) have been used to measure uTF procoagulant activity in disease states (Carty et al, 1990; Lwaleed et al, 1999), glomerulonephritis (Lwaleed et al, 1997a) and solid tumors (Lwaleed et al, 1997b). The effect of renal function on uTF measurements was studied by Lwaleed et al. (1998).

Changes in TF activity, and particularly uTF activity, have been demonstrated in several disease states. The uTF PCA, on the other hand, was slightly raised in patients with hyperthyroidism and was significantly increased in patients with Glomerulonephritis and cancer (Carty, 1990; Adamson 1992 & 1993; Colluci, 1991; Lwaleed 1996 & 1997, are examples). The uTF procoagulant activity was normal in thromboembolic and liver disease and markedly decreased in patients with parenchymal renal diseases and diabetes mellitus (Qi H et al, 1996).

Although the mechanisms of TF elevation (including uTF elevation) in cancer patients are poorly understood, its level may be of clinical importance. Carty et al. reported increased uTF activity in patients with colorectal cancer, inflammatory bowel disease, and breast cancer. Although patients with benign colorectal and breast disease also had increased levels compared with normal controls or patients with rheumatoid arthritis, these were less than those in the malignant group. In patients with an abnormal colonoscopy 88 per cent had abnormal uTF levels, compared with 24 per cent of patients with normal colonoscopy. Adamson et al. showed on average that higher levels of uTF in patients with malignant disease of the bladder and prostate than in controls and patients with benign prostatic hypertrophy. Lwaleed et al. showed no significant difference between normals, surgical controls or patients with benign (non-inflammatory) conditions. However, the controls showed significantly lower uTF levels than those with malignant or benign (inflammatory) conditions. Spillert and Lazaro, Østerud et al., and Edwards et al. suggested that inflammatory conditions interfere with the host immune response, which leads to an increase in monocyte TF expression, a mechanism which could similarly explain uTF elevation in these conditions. However, Carty et al. found that subjects with rheumatoid arthritis, all of whom had active disease, showed generally normal uTF levels. This suggests that uTF does not behave simply as an acute phase reactant.

TF activity (including uTF activity) has shown some correlation to histological tumor grading and/or staging (breast, bladder, prostate, and colorectal cancer), bone scan status (prostate cancer), and serum prostate-specific antigen levels (prostate cancer) (Lwaleed, 1997). Patients with recurrent bladder malignancy also showed higher uTF levels than those with a normal check cystoscopy and levels were higher in patients who subsequently died (Adamson, 1992, Lwaleed, 1998).

Problems with uTF Activity Assays for uTF and Characterising Diagnosis and Screening for Disease

Studies using KCA assays suggest that uTF is found in patients with various cancers. None of these studies demonstrate a clear, consistent and highly significant difference between the levels of uTF in normal and diseased subjects, for example a difference between normal subjects and those suffering from benign disease, inflammatory diseases and/or cancer. In all the reports on uTF, there was significant overlap of uTF levels in normal, benign and cancer conditions (Lwaleed et al.). Thus, the chromogenic tests for uTF have not been proven to be an effective quantitative method to use for characterising, screening, diagnosing or monitoring of diseases, for example cancer. The KCA assays are manual assays more suitable for research applications but are less suitable for large scale clinical use and for instrument-based applications.

Assays that measure uTF “activity” also may not reflect the total amount of uTF present in a sample. The extracellular domain of uTF is subject to inactivation by proteolytic cleavage. These cleaved forms will not be measured in functional assays. Additionally, uTF activity can be functionally encrypted by disulfide formation between cyl86 and cys209 in the extracellular domain (Chen V M et al. Biochem 2006;45:12020-8; Ahamed J et al. PNAS 2006;103:13932; Pendurthi et al Blood 2007;3900-8.) Thus, KCA and other functional assays for uTF are likely to under-represent the amount of TF in a sample.

Functional assays that measure the extracellular domain do not discriminate between the unphosphorylated and phosphorylated forms of uTF. Phosphorylation of uTF occurs only in the intracellular (STP) domain.

The STP of TF represents a potentially better cancer biomarker than the extracellular domain. There are four potential isoforms of the STP intracellular domain depending on the phosphorylation state of Ser253 and Ser258 : 1) unphosphorylated STP; 2) phosphoserine 253 (Pser253); 3) phosphoserine 258 (Pser258); 4) phosphoserine 253 and 258 (Pser253/258). One or more of these four isoforms of TF are more highly expressed in cancer than in non-cancer and healthy situations, particularly in the urine but also in other biological fluids such as blood, saliva etc. Thus there is a need for quantitative immune assays that specifically measure the individual isoforms of TF-STP, preferably uTF-STP (Tf found in urine) which may prove to be better and more specific cancer tests than KCA and immunoassays that do not discriminate between unphosphorylated or phosphorylated TF isoforms.

The present invention relates to an isolated antibody or functional fragment thereof, and uses thereof.

The present invention is based in part on the experimental findings that phosphorylated and unphosphorylated peptides corresponding to regions within the TF-STP protein can be used to generate antibodies that react specifically with phosphorylated and unphosphorylated TF-STP isoforms. In particular, said phosphorylated and unphosphorylated TF-STP isoforms are found in the urine (they are uTF-STP isoforms).

The composition of an immunoassay may comprise for example polyclonal and/or monoclonal antibodies against TF-STP peptides or functional variants thereof, comprising anti-TF1 (aa254-263) (SE Q ID NO:1), anti-TF2 (aa245-253) (SEQ ID NO:2), anti-TF3 (aa245-263) (SEQ ID NO:3), anti-TF1-pser²⁵⁸ (SEQ ID NO:4)and anti-TF2-pser²⁵³(SEQ ID NO:5). Such antibodies are used to develop unique quantitative immunoassays (e.g. ELISAs) capable of selectively measuring the various unphosphorylated and phosphorylated isoforms of TF-STP in biological fluids including urine. The invention further concerns compositions and methods for developing immunoassays and ELISAs for quantitating specific TF-STP isoforms, especially phosphorylated isoforms as described above, in biological fluids including urine. The invention also concerns methods for manufacturing kits comprising the antibodies of the invention, diagnostic kits, and methods for screening, monitoring and diagnosing cancer and other diseases by quantitating TF-STP isoforms in biological fluids including urine.

For example, methods for quantifying TF-STP isoforms in a biological sample may comprise the steps of: (a) contacting and incubating the biological sample with a capture reagent immobilized to a solid support, wherein the capture reagent is a polyclonal or monoclonal antibody that recognizes one antigen site on TF-STP; (b) separating the biological sample from the immobilized capture reagents; (c) contacting the immobilized capture reagent-target TF-STP isoform complex with a detectable polyclonal or monoclonal antibody that binds to a second antigen site on TF-STP; and (d) measuring the level of TF-STP isoform bound to the capture reagents using a detection means for the detectable antibody. In certain embodiments, quantitation of the TF-STP isoform is achieved by using natural tissue factor protein, recombinant tissue factor protein or synthetic peptides corresponding to amino acid sequences in the TF-STP as the comparison standard to determine the amount or relative amount of TF-STP isoform. In certain embodiments, the biological fluid is preferably unfractionated or fractionated urine but can also be plasma, cerebral spinal fluid, blood, serum, tumor samples or tumor lysates which are isolated from a human subject. Wherein said biological fluid is urine, TF-STP isoforms may be referred to as uTF-STP isoforms.

Previous results have examined the use of TF activity as a marker for disease and/or the stage of type or grade of a disease, in particular of cancer. For example, Lwaleed et al. found poor differentiation based on uTF activity levels between patients with various cancers and control patients. Lwaleed et al found an elevated uTF activity level on average for the cancer group relative to the control group, but the measured uTF activity levels for a large fraction of the cancer group were at levels corresponding with the bulk of the control group.

The present invention described herein relies upon the measurement of the various isoforms of the TF-STP region rather than measurement of the extracellular domain of TF. In the current embodiment quantitative immunoassays are used to quantitate the various TF-STP isoforms in biological samples. The novel ELISA assays used to quantitate TF-STP isoforms find good diagnostic utility to distinguish between cancer and control patients. Thus, quantitation of TF-STP isoforms has been validated as a desirable diagnostic tool for cancer diagnosis with appropriate levels of sensitivity and specificity, in particular wherein said isoforms are found in urine.

In particular the quantitation of unphosphorylated and phosphorylated forms of TF-STP in biological samples (tissue or fluids) can be used to determine the presence of cancer in the body. Methods are provided for using the quantitation of TF-STP isoforms in biological fluids for diagnosis, for screening undiagnosed populations for cancer, for monitoring progression and treatment in previously diagnosed cancer patients and for determining the grade and stage of cancer. Quantitation of TF-STP isoforms can be performed using non-immunological methods such as mass spectroscopy, HPLC, gas chromatography, or by immunological methods employing antibodies that specifically bind to TF-STP isoforms. Antibody-sandwich ELISA immunoassay methods for detecting various TF-STP isoforms in biological fluids have been described previously and are the preferred method for quantitation of the TF-STP isoforms.

In preferred embodiments, a sample from a subject is tested for quantitation of unphosphorylated and phosphorylated forms of TF-STP. Preferably the subject is a human. In certain embodiments of the invention, the human subject is a person with an undiagnosed cancer or disease or is a patient diagnosed with cancer. The type of cancer can be solid cancers including but not limited to bladder, colon, breast and prostate cancers or lymphoid cancers including but not limited to leukemias and lymphomas of all types. Based on the measurements from samples from healthy control groups an expected range of readings for a healthy individual can be set for TF-STP isoform(s). Preferably based on fractionated or unfractionated urine samples healthy control groups, an expected range of readings for a healthy individual can be set for uTF-STP isoform(s).

The specimen used for determination of the amount of TF-STP isoform may be tissue or biological fluid obtained from the human subject. The tissue may be processed in such a way to make an soluble extract containing the TF-STP isoform from which the amount of TF-STP isoforms can be determined. The biological fluid may be blood, urine, cerebral spinal fluid, saliva, serum obtained from a human subject. The preferred biological fluid is urine.

In certain embodiments of the invention, the amount of unphosphorylated or phosphorylated TF isoform is determined to be abnormally high or abnormally low as compared to a healthy control group. A reading above or below the normal range can then be flagged as an unusual reading. Based on a reading of an abnormal TF-STP level, additional tests can be performed on the patient to further the diagnosis. The cut-off of the normal range can be selected to balance the number of false negative readings and false positive readings. For example, selection of a lower cut-off of the “normal” range results in a larger number of false positive readings and a smaller number of false negative readings, and selection of a larger cut-off value of the “normal” range will result in a larger number of false negative readings and a smaller number of false positive readings. For example, a cut-off value can be set such that at least 75% of cancer patients have a TF-STP concentration greater than at least 75% of a healthy adult control population as measured by the ELISA assay, or in other embodiments a concentration of TF-STP greater than 90% of a healthy adult control population. A person of ordinary skill in the art will recognize that additional ranges of cut-off values within the explicit range above are contemplated and are within the present disclosure and that absolute values may vary with specific assay methods.

The methods of the present invention can comprise, comparing the detected level of one uTF-STP isoform against a reference level of said uTF-STP isoform, wherein the reference level has been obtained through the ELISA measurement of the level of uTF-STP isoform detected from urine sample of healthy individuals to determine if a patient should be flagged as a likely suffering from a type of disease, such as a cancer, or to determine the stage, grade or type of disease.

In some embodiments of the invention, the quantitation of two or more TF-STP isoforms may be used for characterising, diagnosis, screening, monitoring, grading and staging for a disease, such as cancer. The methods of the present invention can comprise, comparing the detected level of two or more TF-STP isoforms against a reference level of TF-STP of each isoform, wherein the reference level has been obtained through the ELISA measurement of the level of each uTF-STP isoform detected from samples of healthy individuals. Preferably said TF-STP isoforms are quantitated in urine, from both the subject and healthy individuals (controls). In such instances, the quantitative sum, the quantitative difference or the ratio of different TF-STP isoforms in the biological fluid in comparison to the amounts in the normal range may be used to determine the presence of cancer in a sample. Certain algorithms for using the quantitative results from two or more TF-STP isoforms in a sample can be employed to determine the presence, the type, the stage and or the grade of cancer is provided.

Assay Types

There are many immunoassay formats that have been described including assays preferably performed in a clinical laboratory and assays that can be performed on site or at the point of care (POC). This invention is not meant to be limiting in the ELISA method that is used for quantitation of uTF-STP isoforms. The antibodies described herein can be used in other immunoassay formats that utilize immunobeads, paper strips, biochips, etc. An ELISA assay is designed to result in the quantitative binding of the target analyte to a capture antibody adhered to a fixed structure, such as a well of a microtiter plate. Addition of a second labeled detection antibody is added to react with the antibody-captured antigen. In one embodiment, the capture antibody is a polyclonal or a monoclonal antibody. In certain embodiments, the detection antibody is a polyclonal or a monoclonal antibody. In certain embodiments, the detectable antibody is labeled with biotin and the detection means is streptavidin-poly-horseradish peroxidase labeled and 3,3′5,5′-tetramethyl benzidine is the coloured substrate. The detection product is visibly detectable through a colour change of the substrate. Specifically, for some embodiments, the absorption of light at a selected frequency as performed in a microtiterplate spectrophotometric reader can be used to measure the amount of detectable product. A substrate for the enzyme is incubated with the washed well with bound detection antibodies for an appropriate time to allow the substrate to react with the enzyme. Embodiments of particular interest, the substrate reacts with the enzyme to form product compound with a specific colour that can be detected with optical measurements. A desirable substrate for the horseradish peroxidase is tetramethyl benzidine, although other substrates can be used as described below. The detection substrate can be incubated with the bound enzyme for a development incubation time prior to measurement. A stop solution can be added optionally at the end of the development incubation time to halt further reaction of the substrate to allow time for the optical measurement without altering the quantification of the reaction. The colour produced from the reaction of the substrate with the bound enzyme can be read at an appropriate light wavelength for the product, for example, using a plate reader or the like.

Kits are also provided that utilize selective pairs of antibodies for specifically measuring TF isoforms. In one embodiment, the invention provides for an ELISA against the unphosphorylated form of TF and uses a capture reagent monoclonal antibody, which is murine monoclonal antibody anti-TF2 MAb 2G6, and a detectable antibody, which is anti-TF3 (anti-SEQ ID NO:3) PAb-biotin labelled. In yet another embodiment, the invention provides an ELISA for quantitation of uTF-STP-pser²⁵⁸ isoform (SEQ ID NO:4 or SEQ ID NO:7 or SEQ ID NO:8). In this ELISA the capture antibody is anti-TF2 Pab (anti-SEQ ID NO:2) and the detection antibody is anti-TF1-pser²⁵⁸ Pab-biotinylated (antibody ab62251, anti SEQ ID NO:4)). In yet another embodiment, the invention provides an ELISA for quantitation of uTF-STP-pser²⁵³ isoform (SEQ ID NO:5, SEQ ID NO:6 or SEQ ID NO:8). In this ELISA the capture antibody is uTF-STP-pser²⁵³ hybrid MAb 680 (anti SEQ ID NO:5, 6, or 8) and the detection antibody is anti-TF2-Pab-biotinylated (anti SEQ ID NO:2).

In certain embodiments, the kit further comprises a solid support for the capture reagents. For example, the capture reagents can be immobilized on the solid support (e.g., a microtiter plate). In certain embodiments, the kit further comprises a detection means (e.g., colourimetric means, fluorimetric means, etc.) for the detectable antibodies. In certain embodiments, the kit further comprises recombinant tissue factor (rTF), synthetic peptide TF3 (SEQ ID NO:3), synthetic peptide TF1-Pser²⁵⁸ (SEQ ID NO:4) and TF2-Pser²⁵³ (SEQ ID NO:5) as the antigen standards for the respective kits.

The quantification of the TF-STP isoform in a sample from a patient, in particular in a urine sample, relies on the comparison of the measured product of the detection substrate against values of standard solutions plotted in a standard curve. The standard solutions can be formed with concentrations of TF-STP synthetic peptides or rTF over an appropriate range of concentrations to be meaningful for the designed ELISA assay. The assay measurements based on the standard samples are then plotted as a function of the TF-STP or rTF concentrations in the standards to obtain the standard curve. An optical measurement then from an assay on a patient's sample, such as a urine sample, can then be used to determine a TF-STP concentration based on the standard curve by reading a TF-STP concentration for a particular optical measurement. The standard samples should be obtained from the same biological fluid as the patient sample, for example from urine.

In a further aspect, the invention pertains to a method for performing the ELISA assay described herein on biological fluids such as blood, plasma, serum, cerebral spinal fluid or urine from a patient. The preferred biological fluid is urine. Full length TF is known to be present within lipid microparticles of various sizes and weights in the urine. Methods for measuring uTF-STP isoforms present in different microparticle fractions of the urine sample are also described. A urine sample for testing in the ELISA is prepared by adding a sample buffer directly to the urine sample (unfractionated urine sample) and then measuring the uTF-STP isoform(s) in the sample using an appropriate ELISA method. The uTF-STP isoforms can also be measured in a “fractionated” urine sample. As used herein, the term “fractionated urine” refers to a urine sample which has been prepared by centrifuging the urine sample so that the larger/heavier microparticles in the urine can be collected. Most preferably, said centrifuging occurs at about 14,000×g to pellet the larger/heavier microparticles in urine. The supernatant is removed and a sample buffer is added to the pellet containing heavy/larger microparticles to make a solution. The solubilized microparticles are then run in the ELISA to quantitate the uTF-STP isoforms. The ability to use the urine samples with reduced or eliminated pre-processing of the urine samples reduces processing steps while obtaining a desirable assay with good specificity and sensitivity. Thus, the assay herein is well suited for adaptation to a commercial assay that can provide valuable diagnostic capabilities to a broad patient population. The assays described herein can be adapted for commercial application for low cost and relatively high volume processing. Also, if desired, the assays can be adapted for incorporation into automated systems for performing the measurements. The relative speed, efficiency of processing, short incubation times, high sensitivity and/or high selectivity are consistent with performing the assays in a commercial setting.

The results presented herein using the improved ELISA assay for TF-STP isoforms find good diagnostic utility of the TF-STP markers to distinguish between cancer and control patients. Thus, the TF-STP ELISA described herein provide novel laboratory devices for cancer diagnosis, in particular by using urine samples.

Anti-TF-STP Polyclonal Antibodies

Three synthetic peptides homologous to amino acid sequences in TF-STP were made using solid phase methods well known:

(SEQ ID NO: 1)
1) Peptide TF1 CKENSPLNVS (aa254-263);
(SEQ ID NO: 2)
2) Peptide TF2 CRKAGVGQSW (aa245 - 253)
and
(SEQ ID NO: 3)
3) Peptide TF3 CRKAGVGQSWKENSPLNVS (aa245 - 263).

The synthetic peptides were conjugated via the n-terminal Cysteine residue to keyhole limpet hemocyanin (KLH) protein using a bifunctional thioether crosslinker. Polyclonal antibodies to the conjugated peptides were raised in rabbits by immunizing the rabbits subcutaneously with Freund's adjuvant at monthly intervals. Blood was drawn from the rabbits and serum was produced. Antibodies were purified from the serum of the rabbits by affinity purification on Protein G column using standard techniques. The presence of polyclonal antibodies to anti-TF-STP peptides in the serum were determine by solid phase immunoassay whereby the KLH-conjugated peptide or the unconjugated peptide was bound to a microtiter well. The antiserum was added to the well, incubated for about one hour and the well was washed. A horseradish peroxidase labeled anti-rabbit IgG antibody was added to the well and incubated for an hour. The wells were washed and coloured HRP substrate TMB was added to the well. The enzymatic reaction was stopped by addition of sulfuric acid and the yellow colour was measured at OD450 nm. The presence of yellow colour indicated the binding of anti-TF STP antibodies to the solid phase. High titer antibodies to TF1, TF2 and TF3 were detected in the purified rabbit IgGs.

As TF and uTF (TF found in urine) and structurally and chemically identical, anti-TF STP antibodies are also anti-uTF STP antibodies.

Anti-TF-STP Monoclonal Antibodies

Synthetic peptides homologous to amino acid sequences in TF-STP were made using solid phase methods well known in the art: 1) Peptide TF1 CKENSPLNVS (aa254-263) (SEQ ID NO:1); 2) Peptide TF2 CRKAGVGQSW (aa245-253) (SEQ ID NO:2). The synthetic peptides were conjugated via the n-terminal Cysteine residue to keyhole limpet hemocyanin (KLH) protein using a bifunctional thioether crosslinker. The KLH-TF1 and KLH-TF2 conjugates were used to immunize mice for development of hybridomas using standard techniques and protocols known in the art. Hybridomas producing monoclonal antibodies were screened and selected to react preferentially with the TF1 or TF2 peptides, respectively. Hybridomas were expanded and grown up in vitro in culture medium. Monoclonal antibodies produced by the hybridomas were purified from the culture medium by affinity chromatography on Protein G columns. Two clones against TF1 peptide were produced 9D11 1H1 2E11 and 9D11 2E3 1B4. Two clones against TF2 peptide were produced 1B9 1A5 2B7 and 1 B9 2B10 2G6.

Phosphopeptide 3 TF1-Pser²⁵⁸ CKEN(pS)PLNVS (SEQ ID NO:4)and phosphopeptide 4) TF2-Pser²⁵³ CRKAGVGQ(pS)W (SEQ ID NO:5). [Note: pS represent phosphoserine residues.] were conjugated to KLH and used as immunogens. The KLH-TF1PSer²⁵⁸ and KLH-TF2PSer²⁵³ conjugates were used to immunize mice for development of hybridomas using standard techniques and protocols known in the art for monoclonal antibodies against the phosphoserine 253 and phosphoserine 258 isoforms of uTF-STP. Hybridomas producing monoclonal antibodies were screened and selected to specifically react preferentially with the phosphoserine portion of each of the two respective peptides. Hybridomas were expanded and grown up in vitro in culture medium. Two anti-TF1-Pser²⁵⁸ (SEQ ID NO:4) clones were selected and designated as MAb clone 6A10 and MAb clone 4D5. Monoclonal antibodies of the IgG isotype produced by the hybridomas were purified from the culture medium by affinity chromatography on Protein G columns.

One anti-TF2-Pser²⁵³ clone (anti SEQ ID NO:5) 4A6-4B5-1E2 was selected and produced a monoclonal antibody of the IgM isotype. The IgM Fab region was genetically engineered onto the backbone of a murine IgG2b antibody using methods known in the art. The hybrid IgG anti-TF2-pser²⁵³ M680 (anti-SEQ ID NO:5) was produced via transfection of HEK cells by plasmids containing the genetically engineered light and heavy chains. The hybrid antibody protein was purified using affinity chromatography on Protein G column. The hybrid MAb had molecular weight of approximately 150,000 kD and specifically bound to TF-STP-pser²⁵³ (SEQ ID NO:5) peptide.

Standards for TF-STP ELISAs

The standards for the various TF-STP ELISAs described herein were comprised of synthetic peptides and recombinant tissue factor (rTF). The standards for ELISAs measuring unphosphorylated peptides comprised unphosphorylated peptide TF3 CRKAGVGQSWKENSPLNVS (SEQ ID NO:3) or full length rTF protein containing the native intact unphosphorylated STP region (Haematologic Associates, VT). The standards for ELISAs measuring TF-STP-Pser²⁵⁸ isoform (SEQ ID NO: 7) the TF3-Pser²⁵⁸ CRKAGVGQSWKEN(pS)PLNVS (SEQ ID NO:7) synthetic peptide was used as the standard. The standards for ELISAs measuring uTF-STP-Pser²⁵³ isoform (SEQ ID NO:6) the TF3-Pser²⁵³ CRKAGVGQ(pS)WKENSPLNVS (SEQ ID NO:6) synthetic peptide was used as the standard. The double phosphorylated TF3-Pser²⁵³/pser²⁵⁸ synthetic peptide CRKAGVGQ(pS)WKEN(pS)PLNVS (SEQ ID NO:8) was also used as a standard in some experiments.

ELISA Assay Kit

Suitable ELISA assay components can comprise a capture antibody chosen from any one of the appropriate anti-TF-STP polyclonal or monoclonal antibodies described above which is immobilized on a microtiter well. The detection antibody can be selected from any one of the other monoclonal and polyclonal antibodies to TF-STP that would form the proper partner for detection of the isoform desired. With respect to the capture and detection antibodies, the desired concentrations can also depend on the particular format of the assay, the nature of the well or other capture format features and the like. The detection antibody can be biotinylated, and the signal generating enzyme, e.g., horse radish peroxidase, can be conjugated to avidin or streptavidin. A poly-hrp-streptavidin conjugate can be used to increase the signal strength. Biotin is a natural cofactor with a strong binding affinity to proteins avidin and streptavidin. Streptavidin and avidin bind with high specificity to biotin, allowing for the signal generation upon addition of the colourimetric substrate, e.g., TMB. Other similar detection antibody formats can be used as desired. Suitable streptavidin peroxidase enzyme substrates are available commercially. For example, 2,2′-azino-bis(3-ethylbenzthiazoline-6-sulfonic acid) reacts to form a green product and 3,3′5,5′-tetramethyl benzidine (TMB) reacts to form a blue reaction product. In particular, TMB has a high sensitivity, which can be desirable for the ELISA assay described herein. For TMB, sulfuric acid can be used as a stop solution. After application of the stop solution, the TMB product becomes yellow and can be measured at 450 nm. Alternatively, no stop solution can be used, and the blue colour product can be measured at 650 nm.

A kit for performing the assay can comprise the core components, i.e., the anti-TF-STP capture antibody and the anti-TF-STP-biotin detection conjugate and the streptavidin-poly-hrp conjugate. Optionally, a kit can comprise one or more additional components for the assay as summarized above. Reagents can be supplied in at the desired dilution or at a known concentration that can then be further diluted to a desired dilution. For performing the assay, additional reagents are used, such as wash solutions, and the like, which generally are sufficiently pure and have appropriate compositions so as to not interfere with the assay. Wash solutions and the like can comprise purified water, saline, buffer, combinations thereof or the like, and these solutions are generally commercially available. A kit can comprise wash solutions if desired. A kit can further comprise instructions for the proper performance of the assay. The assay can be designed for performance on an automated system, as described further below, and the kit components can be appropriately packed for such an application. The components of the kit may or may not be physically packaged together, although packaging certain components, such as the core components may be convenient.

Sample Handling Methods and Testing

An ELISA assay can then be performed on a biological fluid sample using the reagents outlined above. The preferred sample is human urine. The basic format of the ELISA assay described herein involves capture of the TF-STP isoform(s) in the sample, especially in a urine sample, by the immobilized anti-TF-STP antibody and the detection of the captured TF-STP isoform by the second anti-TF-STP antibody-biotin/streptavidin-hrp detection complex. The TF-STP isoforms are usually found in microparticles of various sizes—large and small—in the sample. The microparticles need to be disrupted and solubilized for optimal reaction with the antibodies in the ELISA. This is accomplished by the addition of a sample buffer that has a detergent as one component. The detergent can be of various kinds on ionic and non-ionic detergents including but not limited to Triton X-100, Tween 20, NP-40, CHAPS and sodium dodecylsulfate (SDS). The buffer may be of various compositions and pH's depending on the requirements of reaction of the TF-STP isoform with the capture and detection antibody in the ELISA.

Samples obtained from a patient can be transferred to an appropriate laboratory for performance of the assay. For example, urine samples can be frozen and stored at −20° C. to −120° C. before being subjected to the ELISA. Furthermore, urine samples can be obtained as a small aliquot from a 24 hour urine collection. The samples can be pre-cleared of debris by centrifugation at 400×g for 20 minutes to remove any solid contaminants. This can be done prior to freezing or after freezing when the sample is ready to be assayed in the ELISA.

When using urine as the sample, the TF-STP isoform can be measured directly by mixing equal volumes of 2× sample buffer and the pre-cleared urine sample together and then adding the sample to the ELISA well. This method measures the uTF-STP in unfractionated urine and represents the total amount of uTF-STP in all the microparticles in a urine sample. Another method for preparing the sample is to measure the uTF-STP isoforms in the large microparticles in urine. This is accomplished by centrifuging the pre-cleared urine sample at 14,000×g for 20 minutes to 45 minutes. This can be done using a typical bench-top microcentrifuge and spinning the urine sample (1.5-2.0 ml) in a plastic microcentrifuge tube. The supernatant liquid is removed and the pellet saved. A small amount of 1× sample detergent buffer is added to the pellet at the bottom of the tube and the tube is vigorously mixed to dissolve the pelleted large microparticles in the sample buffer. This is referred to as the fractionated urine sample and represents the heavy or large microparticles in urine.

Performance of the ELISA

Biological samples are measured for their content of TF-STP and for individual isoforms including unphosphorylated TF-STP (SEQ ID NOs:1, 2, and 3), TF-STP-Pser253 (SEQ ID NO:5, 6, and 8) and TF-STP-Pser258 (SEQ ID NOs:4, 7 and 8). The ELISAs that quantitate the specific isoforms have been previously described. The general outline of the ELISA is performed as follows. Standards are diluted into appropriate sample buffer. Samples are preferably prepared either as unfractionated samples or as the fractionated samples, most preferably wherein said sample are urine samples (see above). The standards and samples are added to antibody-coated microtiter plate wells. The microtiter plate is incubated for 16 hours overnight at ambient temperature with gentle shaking. The plate is washed four times with wash buffer. Biotinylated-detection antibody at appropriate dilution in buffer solution is added to the microwells and incubated for at ambient temperature. The plate is washed four times with wash buffer. Streptavidin-polyHRP reagent is diluted in buffer. The plate incubated with shaking for 1.5 hours. The plate is washed 4 times with wash buffer. HKTMB coloured substrate (from Moss) 100 ul is added. The plate is incubated with shaking as before for 10-20 minutes. The reaction stopped by adding 10 ul of 1 N sulfuric acid stop solution. Plate read in a microtiter spectrophotometer plate reader at 450 nm. A standard curve is created by plotting the concentration of the standards verses the OD 450 nm. A linear or polynomial curve fitting program is used to create a standard curve. The concentration of the TF-STP isoform in the sample is interpolated from the OD of the sample from the standard curve. This is a general outline of performing the ELISA. One skilled in the art can modify the assay to suit various needs. Other immunoassay formats can be used to quantitate TF-STP in samples. Such assays include lateral flow, point of care, radioimmunoassay, competitive immunoassays, immunoprecipitation, latex immunoassays, etc.

Buffers and Detergents

Many pairs of antibodies, detergents, salts and buffer conditions were tested in the development of the disclosed ELISAs. TRIS (tris(hydroxymethyl) aminomethane), phosphate, MOPS (3-(N-morpholino)propanesulfonic acid) buffers ranging in pH from 6-8.5 were investigated for all ELISAs. Furthermore, NaCl salt concentrations in the buffers ranging from 0-0.5M were tested. Optimal NaCl levels were found to be around 0.14 M.

Different detergents were also tested, including Triton X-100 (p-(1,1,3,3-tetramethylbutyl)-phenyl ether), Tween 80 (Polyoxyethylene (20) sorbitan monooleate), CHAPS (3-[(3-cholamidopropyl)dimethylammonio]-1-propanesulfonate) and sodium dodecylsulfate (SDS). Combinations of the various parameters antibodies, detergent, salt and buffer conditions were evaluated.

ELISA for unphosphorylated TF. The optimal pair of antibodies for this ELISA were found to be the anti-TF2-Mab clone 1B9 2610 2G6 coated on the plates as the capture antibody and the rabbit polyclonal anti-TF3-biotinylated as the detection antibody. Reversing the pair by using the Pab TF3 as the capture antibody and the TF2-Mab-biotinylated as the detection antibody does not work, in that by the phrase “does not work” as used herein in relation to ELISAs means that there was no useful colour signal developed, the background was too high or there was not the desired specificity for the target TF-STP isoform.

Other antibodies were tried as a detection antibody paired with anti-TF2 Mab as capture antibody without success. These antibodies included anti-TF2 Pab, anti-TF1 Pab, anti-TF1-Mab.

The optimal detergent for this assay was determined to be CHAPS. The CHAPS concentration can be used between 1 mM and 10 mM with the optimal concentration of 4 mM. The buffers useful in this assay can range from pH 6.0-8.5. The optimal pH was determined to be phosphate buffer between pH 7.2-7.4.

ELISA for TF-Pser258. The optional pair of antibodies for this ELISA were the anti-TF2-Pab coated on the plates as the capture antibody and the anti-TF-Pser258 Pab-biotin (abcam #62251, Cambridge, Mass.) as the detection antibody. The Anti-TF1-Pser258 Mab Clone 6D1-1D7-1E9 can also be used as the detection antibody with anti-TF2-Pab as the capture antibody. Reversing the pair by using the Pab TF2-biotin as the detection antibody and the anti-TF-Pser258 Pab as the capture antibody does not work.

Other antibodies were tried as a capture antibody with anti-TF-Pser258 Pab as detection antibody without success. The other capture antibodies tried included ant-TF3 Pab, anti-TF2-Mab, anti-TF1 Pab, anti-TF1 Mab.

The optimal detergent for this assay was determined to be SDS. The SDS concentration can be used from 0.01-0.1% with the optimal concentration being 0.02%. Other detergents including Triton X-100, Tween 80, and CHAPS were determined to be not appropriate for this assay. The pH of buffers useful in this assay can range from pH 6.0-8.5. The optimal pH was determined to be phosphate buffer between pH 7.2-7.4.

ELISA for TF-Pser253. The optimal pair of antibodies for this ELISA were found to be the Anti-TF2-Pser253 IgG hybrid Mab Clone F215 4A6-4B5-1E2 coated on the plates as the capture antibody and the anti-TF2-Pab-Biotin as the detection antibody. Reversing the pair by using the anti-TF2 Pab as the capture antibody and the anti-TF2-Pser253 hybrid Mab-biotinylated as the detection antibody does not work.

Other antibodies were tried as a detection antibody with anti-TF2-Pser253 hybrid Mab as capture antibody without success. These other detection antibodies tried included anti-TF3 Pab, anti-TF2-Mab, anti-TF1 Pab, anti-TF1 Mab.

The optimal detergent for this assay as determined to be SDS. The SDS can be used from 0.01-0.1% with the optimal concentration 0.02%. The detergents Triton X-100, Tween 80, and CHAPS were determined to be not appropriate for this assay. The buffers useful in this assay can range from pH 6.0-8.5. The optimal pH was determined to be phosphate buffer at pH 7.4.

Using in an ELISA a commercial antibody [Phospho(Ser) PKC Substrate antibody #2261 (Cell Signaling Technologies, Danvers, Mass.)] to a consensus PKC phosphoserine substrate sequence that crossreacts with the TF2-Pser253 sequence was also attempted. The commercial anti-TF-Pser253 Antibody 2261 was tried as the capture and as the detection antibody paired with various of our other anti-TF antibodies, all without success. Additionally, the anti-TF2-Pser253 IgM Mab from which the hybrid anti-TF-Pser253 IgG antibody was made also did not work in any other configurations tried. It is likely that the IgM is too large a molecule (750K MW) and causes steric hindrance in the assay and prevents binding of the second antibody in the two-site ELISA format.

Determination of Cancer

The assay measurements described herein can be used as a tool to characterise a disease, in particular the type, stage, grade or presence of a disease. Preferably, the disease is cancer. Preferably said methods are carried out in vitro. The results presented in the examples demonstrate the ability of an assay, most preferably a urine assay (though use of any biological sample is envisaged) to provide meaningful differentiation between a control group of patients and patients with cancer. The ability to have a tool that is non-invasive, relatively inexpensive and routine with respect to sample collection can lead to earlier diagnosis, characterising and/or grading of a disease (such as determining the severity of a disease) with a corresponding significant potential for improvement in outcomes and a corresponding reduction in total medical costs and social costs.

Changes in TF-STP (and in particular phosphorylated TF-STP) levels can also be useful in monitoring patient treatment, and screening undiagnosed populations for cancer. In particular, wherein said TF-STP level is elevated and/or elevated phosphorylated TF-STP levels are found in urine. Elevated TF-STP levels have also been correlated with certain inflammatory diseases, and the availability of a commercial assay may also benefit the diagnosis and tracking of these diseases. The term “elevated” in this context means increased compared to a control, preferable a control polulation.

The results below demonstrate the ability to reasonably diagnose of an elevated probability of the presence of cancer in the body based on measurements of uTF-STP levels in urine. The cancer and control groups may not have completely separate levels of uTF-STP, but with the effective assays the extent of false negative reading and false positive readings are reasonable based on the analyses described herein.

Based on the measurements of control groups, an expected range of readings for a healthy individual can be set. An elevated reading above the normal range can then be flagged as an unusual reading. Based on a reading of an elevated TF-STP level, particularly uTF-STP level in a urine sample, additional tests can be performed on the patient to further the diagnosis. The cut-off of the normal range can be selected to balance the number of false negative readings and false positive readings. For example, selection of a lower cut-off of the “normal” range results in a larger number of false positive readings and a smaller number of false negative readings, and selection of a larger cut-off value of the “normal” range will result in a larger number of false negative readings and a smaller number of false positive readings. For example, a cut-off value can be set such that at least 75% of cancer patients have a TF-STP concentration ratio greater than at least 75% of a healthy adult control population as measured by the ELISA assay, or in other embodiments a concentration ratio of 90% of a healthy adult control population. A person of ordinary skill in the art will recognize that additional ranges of cut-off values within the explicit range above are contemplated and are within the present disclosure and that absolute values may vary with specific assay methods.

Antibody Sequences

In the current invention, antibodies have been created which are specific to TF and fragments thereof, preferably human TF, and most preferably human TF found in urine (uTF). Preferably said antibodies are specific to the c-terminal of TF, such as human TF (or human uTF).

In a most preferred embodiment, the antibodies of the current invention are specific for the signal transduction peptide (STP) region of TF, which is structurally and chemically identical to uTF-STP.

In particular, antibodies have been created which are specific to the polypeptides disclosed herein, such as SEQ ID NOs:1, 2, 3, 4, 5, 6, 7, 8, 9, 10, 11, 12, 13 and 14.

In particular, antibodies have been created which are specific to the polypeptides having at least 90% identity to SEQ ID NOs:1, 2, 3, 4, 5, 6, 7, 8, 9, 10, 11, 12, 13 and 14.

In a preferred embodiment, antibodies have been created which are specific to the polypeptides having at least 95% identity to SEQ ID NOs:1, 2, 3, 4, 5, 6, 7, 8, 9, 10, 11, 12, 13 and 14.

In a preferred embodiment, antibodies have been created which are specific to the polypeptides of SEQ ID NOs:1, 2, 3, 4, 5, 6, 7, 8, 9, 10, 11, 12, 13 and 14 or polypeptides having 1 or 2 or more substitutions, additions, deletions or insertions thereto, in particular wherein the addition is a cysteine residue, especially a c-terminal cysteine residue.

In one embodiment, antibodies have been created which are specific to phosphorylated TF or fragments thereof, and in particular phosphorylated human TF, most preferably human uTF. Most preferably the phosphorylated amino acid(s) in TF are one or more serine residues. In a most preferred embodiment the phosphorylated serines are at positions 253 and/or 258 of TF (with reference to the position numbering of SEQ ID NO:10). i.e. they are in the STP region of TF.

In a most preferred embodiment, antibodies have been created which are specific to phosphorylated TF of SEQ ID NOs:4, 5, 6, 7, and 8, or polypeptides having at least 90% identity thereto.

In a preferred embodiment, antibodies have been created which are specific to phosphorylated TF of SEQ ID NOs:4, 5, 6, 7, and 8, or polypeptides having at least 95% identity thereto.

In a most preferred embodiment, antibodies have been created which are specific to phosphorylated TF of SEQ ID NOs:4, 5, 6, 7, and 8, or polypeptides having 1 or 2 or more substitutions, additions, deletions or insertions thereto.

SEQ ID NO:11 shows the heavy chain variable region of the antibody specific for SEQ ID NO:5, wherein SEQ ID NO:5 has a phosphorylated serine at position 253.

SEQ ID NO: 11
MRVLILLWLFTAFPGILSDVQLQESGPGLVKPSQSLSLTCTVTGYSITSD
YAWNWIRQFPGNKLEWMGYISYSGSTSYNPSLKSRISITRDTSKNQFFLQ
LNSVTTEDTATYYCARIRGYLAMDYWGQGTSVTVSSESQSFPNVFPLV

The variable domain is highlighted in BOLD above.

The Complementarity Determining Regions (CDRs) are underlined above, and were determined by the IMGT numbering system (Lefranc, M.-P. et al., Nucleic Acids Research, 27, 209-212 (1999)).

SEQ ID NO:12 shows the amino acid sequence of the light chain variable region of the antibody specific for SEQ ID NO:5, wherein SEQ ID NO:5 has a phosphorylated serine at position 253.

SEQ ID NO: 12
MRSPAQFLGLLVLWIPGSTADIVMTQAAFSNPVTLGTSASISCRSSKSLL
HSNGITYLYWYLQKPGQSPQLLIYQMSNLASGVPDRFSSSGSGTDFTLRI
SRVEAEDVGVYYCAQNLELPPTFGGGTKLEIKRADAAPTVSIFPPSSEQL
TSGGASVVCFLNNFYPK

The variable domain is highlighted in BOLD above.

The Complementarity Determining Regions (CDRs) are underlined above, and were determined by the IMGT numbering system (Lefranc, M.-P. et al., Nucleic Acids Research, 27, 209-212 (1999)).

In one embodiment, antibodies of the invention comprise a polypeptide having at least 90% identity to SEQ ID NO:11.

In a further embodiment, antibodies of the invention comprise a polypeptide having at least 95% identity to SEQ ID NO:11.

In a further embodiment, antibodies of the invention comprise a polypeptide having at least 100% identity to SEQ ID NO:11.

In a preferred embodiment, antibodies of the invention comprise a polypeptide or SEQ ID NO:11, or a polypeptide having one or two or more substitutions, additions, deletions or insertions compared to SEQ ID NO:11.

In one embodiment, antibodies of the invention comprise a polypeptide having at least 90% identity to SEQ ID NO:12.

In a further embodiment, antibodies of the invention comprise a polypeptide having at least 95% identity to SEQ ID NO:12.

In a further embodiment, antibodies of the invention comprise a polypeptide having at least 100% identity to SEQ ID NO:12.

In a preferred embodiment, antibodies of the invention comprise a polypeptide or SEQ ID NO:11, or a polypeptide having one or two or more substitutions, additions, deletions or insertions compared to SEQ ID NO:12.

Biological Sequences

The science of biological sequences is well-known in the art.

The similarity between amino acid or nucleotide sequences is expressed herein and commonly in the art in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Bioi. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol.215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the anti-TF antibodies of the present invention, preferably anti-TF-STP-Pser253 or anti-TF-STP-Pser258, or anti-TF-STP-Pser253Pser258 or a domain thereof (e.g. a VL, VH, CL or CH domain) typically have at least about 75%, for example at least about 80%, 90%, 95%, 96%, 97%, 98% or 99% sequence identity with the original sequence (e.g. a sequence defined above), for example counted over the full length alignment with the amino acid sequence of the antibody or domain thereof using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties).

Proteins with even greater similarity to the reference sequences (for example SEQ ID NO:10) will show increasing percentage identities when assessed by the above-described method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% or 97% or 98% or 99% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Typically variants may contain one or more conservative amino acid substitutions compared to the original amino acid or nucleic acid sequence. Conservative substitutions are those substitutions that do not substantially affect or decrease the affinity of an antibody to it's specific antigen. For example, a human antibody that specifically binds TF-STP-Pser253 (SEQ ID NO:6 or SEQ ID NO:8) may include up to 1, up to 2, up to 5, up to 10, or up to 15 conservative substitutions compared to the original sequence (e.g. as defined above) and retain specific binding to the TF polypeptide. The term conservative variation also includes the use of a substituted amino acid in place of an unsubstituted parent amino acid, provided that antibody specifically binds TF. Non-conservative substitutions are those that reduce an activity or binding to and antigen.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

Production of Antibodies and Nucleic Acids

Nucleic acid molecules (also referred to as polynucleotides, nucleic acids and nucleic acid sequences) encoding the polypeptides provided herein (including, but not limited to antibodies and functional fragments thereof) can readily be produced by one of skill in the art, using the amino acid sequences provided herein, sequences available in the art, and the genetic code. In addition, one of skill can readily construct a variety of clones containing functionally equivalent nucleic acids, such as nucleic acids which differ in sequence but which encode the same effector molecule or antibody sequence. Thus, nucleic acids encoding antibodies are provided herein.

Nucleic acid sequences encoding the antibodies that specifically bind the polypeptides disclosed herein (eg SEQ ID NOs:4, 5, 6, 7, 8, or 14), or functional fragments thereof, can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al.Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is generally limited to sequences of about 100 bases, longer sequences may be obtained by the ligation of shorter sequences.

Exemplary nucleic acids encoding antibodies that specifically bind the polypeptides disclosed herein (eg SEQ ID NOs:4, 5, 6, 7, 8, or 14) or functional fragments thereof, can be prepared by cloning techniques. Examples of appropriate cloning and sequencing techniques, and instructions sufficient to direct persons of skill through many cloning exercises are found see, for example, Molecular Cloning: A Laboratory Manual, 2nd ed., vol. 1-3, ed. Sambrook et al., Cold Spring Harbor Laboratory Press, Cold Spring Harbor, N.Y., 1989); and Current Protocols in Molecular Biology (Ausubel et al., eds 1995 supplement)). Product information from manufacturers of biological reagents and experimental equipment also provide useful information. Such manufacturers include the SIGMA Chemical Company (Saint Louis, Mo.), R&D Systems (Minneapolis, Minn.), Pharmacia Arnersham (Piscataway, N.J.), CLONTECH Laboratories, Inc. (Palo Alto, Calif.), Chern Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Known nucleic acids (such as those encoding known or wild-type antibodies) can be modified to form the antibodies described herein. Modification by site-directed mutagenesis is well known in the art. Nucleic acids can also be prepared by amplification methods. Amplification methods include polymerase chain reaction (PCR), the ligase chain reaction (LCR), the transcription-based amplification system (TAS), the self-sustain sequence replication system (3SR). A wide variety of cloning methods, host cells, and in vitro amplification methodologies are well known to persons of skill.

In one embodiment, antibodies are prepared by inserting DNA which encodes one or more antibody domains into a vector which comprises DNA encoding one or more further antibody domains. The insertion is made so that the antibody domains are read in frame, that is in one continuous polypeptide which contains a functional antibody region.

In one embodiment, DNA encoding a heavy chain constant region is ligated to a heavy chain variable region so that the constant region is located at the carboxyl terminus of the antibody. The heavy chain-variable and/or constant regions can subsequently be ligated to a light chain variable and/or constant region of the antibody using disulfide bonds.

Once the nucleic acids encoding the antibodies of the invention or functional fragment thereof have been isolated and cloned, the desired protein can be expressed in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines.

One or more DNA sequences encoding the antibody or fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. Hybridomas expressing the antibodies of interest are also encompassed by this disclosure.

The expression of nucleic acids encoding the isolated antibodies and antibody fragments described herein can be achieved by operably linking the DNA to a promoter (which is either constitutive or inducible), followed by incorporation into an expression cassette. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, maintenance of the correct reading frame of that gene to permit proper translation ofmRNA, and stop codons.

To obtain high level expression of a cloned gene, it is desirable to construct expression cassettes which contain, at the mmnnum, a strong promoter to direct transcription, a ribosome binding site for translational initiation, and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, SV40 or cytomegalovirus, and a polyadenylation sequence, and can further include splice donor and acceptor sequences. The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be co-transformed with polynucleotide sequences encoding the antibody, labelled antibody, or functional fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Modifications can be made to a nucleic acid encoding a polypeptide described herein without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the antibodies of the present disclosure can also be constructed in whole or in part using standard peptide synthesis well known in the art.

Once expressed, the recombinant antibodies can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The antibodies, immunoconjugates and effector molecules need not be 100% pure. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies disclosed herein. See, Buchner et al., Anal. Biochem. 205:263-270, 1992; Pluckthun, Biotechnology 9:545, 1991; Huse et al., Science 246:1275, 1989 and Ward et al., Nature 341:544,1989

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. Excess oxidized glutathione or other oxidizing low molecular weight compounds can be added to the refolding solution after the redox-shuffling is completed.

In addition to recombinant methods, the antibodies, labelled antibodies and functional fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chern. Soc. 85:2149-2156, 1963, and Stewart et al., Solid Phase Peptide Synthesis, 2nd ed., Pierce Chern. Co., Rockford, III., 1984. Proteins of greater length may be synthesized by condensation of the amino and carboxyl termini of shorter fragments

Methods of forming peptide bonds by activation of a carboxyl terminal end (such as by the use of the coupling reagent N,N′-dicylohexylcarbodimide) are well known in the art.

In one embodiment, the antibodies, nucleic acids, expression vectors, host cells or other biological products are isolated. By “isolated” it is meant that the product has been substantially separated or purified away from other biological components in the environment (such as a cell) in which the component naturally occurs, i.e., other chromosomal and extra-chromosomal DNA and RNA, proteins and organelles. Nucleic acids and antibodies that have been “isolated” include nucleic acids and antibodies purified by standard purification methods. The term also embraces nucleic acids and antibodies prepared by recombinant expression in a host cell as well as chemically synthesized nucleic acids.

Definitions of Terms Used Herein

Antibodies

As used herein, the term “Antibodies” refers to polypeptide ligands comprising at least a light chain or heavy chain immunoglobulin variable region which specifically recognizes and specifically binds an epitope of an antigen, such as for example tissue factor TF-STP isoforms (or uTF-STP isoforms, as TF-STP isoforms found in urine are known), or fragments thereof. Antibodies are typically composed of a heavy and a light chain, each of which has a variable region, termed the variable heavy (VH) region and the variable light (VL) region. Together, the VH region and the VL region are responsible for binding the antigen recognized by the antibody.

Antibodies include intact immunoglobulins and the variants and portions of antibodies well known in the art, provided that such fragments are capable of binding to immunoglobulin ligands or conjugation to moieties which bind other ligands. Antibodies also include genetically engineered forms such as chimaeric, humanized (for example, humanized antibodies with murine sequences contained in the variable regions) or human antibodies, heteroconjugate antibodies (such as, bispecific antibodies), e.g. as described in Uby, J., Immunology, 3rd Ed., W.H. Freeman & Co., New York, 1997. Murine antibodies are those derived from a mouse species, for example mus musculus.

The term “isotype” or “iso-type” is used herein as known in the art, and refers to any of the known categories of antibodies or immunoglobulins determined by their physicochemical properties (such as molecular weight) and antigenic characteristics.

Several isotypes of immunoglobulins are known in the art, for example IgG, IgM, IgE, IgA and IgD (found in mammals), and sub-isotypes such as IgG1, IgG2, IgG3, IgG4, IgG2a or IgG2b. The antibodies of the present invention may comprise any isotype, sub-isotype, combination of isotypes and/or sub-isotypes, variants, portions or fusions thereof.

Each antibody heavy and light chain contains a constant region and a variable region, (the regions are also known as “domains” herein). In combination, the heavy and the light chain variable regions specifically bind the antigen. Light and heavy chain variable regions contain a “framework” region interrupted by three hypervariable regions, also called “complementarity-determining regions” or “CDRs.” herein. The extent of the framework region and CDRs has been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

The fragment antigen-binding (Fab fragment) as referred to herein is a region on an antibody that binds to antigens. It is composed of one constant and one variable domain of each of a heavy chain and a light chain.

The antibodies of the present invention specifically bind (or are specific for) Tissue factor (TF) or an isoform, fragment, domain or portion thereof. In a preferred embodiment, the antibodies of the present invention are specific for human TF, and most preferably human urine TF (uTF). In a preferred embodiment the antibodies of the present invention are specific for the signal transduction peptide of TF (also referred to herein as STP or TF-STP or in the case where it is urine TF, uTF-STP), which in mature human TF (SEQ ID NO:10), comprises amino acids 245-263, with respect to the position numbering of SEQ ID NO:10. In a most preferred embodiment, the antibodies of the present invention are specific for phosphorylates TF, and most preferably phosphorylated human TF, preferably human uTF. In the most preferred embodiment, the antibodies of the present invention, and the antibodies used in the methods of the present invention, are specific for phosphorylated human TF-STP, preferably wherein said phosphorylate occurs at serine 253, serine 258 or both of these serine residues, and most preferably wherein said human TF-STP is found in urine (uTF-STP).

Antibodies which bind TF, preferably TF-STP, may have a specific VH region and VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Thus in embodiments of the present invention, the antibody comprises at least one, two, three, four, five or six CDRs (e.g. 3 heavy chain CDRs and/or 3 light chain CDRs) or at least one variable domain (e.g. a VH or VL domain) derived from an antibody which binds to a TF-STP isoform, preferably a uTF-STP isoform.

As used herein, references to “VH” refer to the variable region of an immunoglobulin heavy chain. References to “VL” refer to the variable region of an immunoglobulin light chain.

As used herein, the term “monoclonal antibody” (also known as an MAb) refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A cell that is produced from the fusion of an antibody producing lymphocyte and a non-antibody producing cancer cell, usually a myeloma or lymphoma, is referred to as a “hybridoma” herein. A hybridoma may proliferate and produce a continuous supply of a specific monoclonal antibody.

As used herein, the term “chimaeric antibody” refers to an antibody comprised of amino acid sequences derived from two different antibodies, which are typically derived from different species. For example, chimaeric antibodies may include human and murine antibody domains, e.g. human constant regions and murine variable regions (e.g. from a murine antibody that specifically binds TF-STP). The antibodies of the current invention may comprise chimaeric antibodies.

Chimaeric antibodies are typically constructed by fusing variable and constant regions, e.g. by genetic engineering, from light and heavy chain immunoglobulin genes. For example, the variable segments of the genes from a mouse monoclonal antibody of one class or isotype, can be joined to the constant segments of another, such as kappa or gamma. In one example, a chimaeric antibody is thus a hybrid protein composed of the variable or antigen-binding domain from a mouse IgM antibody and the constant or effector domain from a mouse IgG antibody, e.g. an Fc (effector) domain from a mouse IgG antibody, although other mammalian species can be used, variable and constant domains can each be derived from different species, or the variable region can be produced by molecular techniques. Methods of making chimaeric antibodies are well known in the art, e.g., see U.S. Pat. No. 5,807,715.

The antibodies of the present invention preferably comprise (i) a heavy chain comprising SEQ ID NO:11, a polypeptide having at least 90% identity to SEQ ID NO:11, preferably at least 95% identity thereto or a functional fragment or variant thereof;

and/or (ii) a light chain comprising the amino acid sequence as defined in SEQ ID NO:12, or a polypeptide having at least 90% identity to SEQ ID NO:12, preferably at least 95% identity thereto or a functional fragment or variant thereof.

An antibody or functional fragment thereof according to the invention is preferably derived or derivable from a biological deposit as described herein below. The biological deposits have been made at the ECACC depositary and consist of the following hybridoma cell lines:—
(1) F182 Anti-TF2 Clone 1B9-2B10-2G6; Accession Number 15072902
(2) F214 Anti-TF pSer253 Clone 4A6-4B5-1E2; Accession Number 15072903
(3) F216 Anti-TF pSer258 Clone 6D1-1D7-1E9; Accession Number 15072904

These biological deposits have been made by the inventors under the Budapest Treaty at European Collection of Authenticated Cell Cultures (ECACC)

Public Health England

Porton Down

Salisbury

SP4 0JG

UK

An antibody of the present invention preferably comprises one or more domains and/or portions derived from an IgM monoclonal antibody.

In one embodiment, an antibody of the present invention preferably comprises the Fab portion of a light and heavy chain of an IgM monoclonal antibody.

In one embodiment, an antibody of the present invention preferably comprises one or more domains and/or portions derived from an IgG, preferably IgGb2.

In one embodiment an antibody of the present invention comprises the Fab portion of a light and heavy chain of an IgM monoclonal antibody fused on to IgGb2 light and heavy chains

In one embodiment an antibody of the current invention is conjugated to a detectable label or a therapeutic agent, an encapsulating agent or a radioactive moiety.

The term “functional fragment” as used herein refers to a fragment, domain or portion of a protein which retains the activity of said protein. A functional fragment of an antibody therefore retains the specific antigen binding ability of the antibody. A functional fragment of an enzyme retains the enzymatic activity of said enzyme.

In particular the term “variant” as used herein refers to a modified molecule such as an enzyme wherein the polypeptide or nucleotide sequence encoding an amino acid has been changed, but the resulting polypeptide has the same activity as the original polypeptide or nucleotide sequence from which it was derived.

The term “antigen” as used herein refers to is any structural substance which serves as a target for the receptors of an adaptive immune response to bind to, in particular for an antibody to bind to. A specific part of an antigen which is recognised by an antibody is known as an “epitope”. Antibodies may bind to different epitopes on the same antigen, preferably at the same time.

The phrases “specific for” or “with specificity”, “specifically bind”, or “with specificity for” as used herein refer to the ability of a molecule, preferably an antibody of the invention, to bind to a limited type of antigen. Preferably, as above, the antibodies of the present invention as specific for tissue factor (TF) or an isoform, portion, domain or fragment thereof.

Diseases

The present invention may be used in methods of characterising, detecting, preventing and/or treating a disease.

As used herein, the phrase “Characterising a disease” encompasses detecting the presence, type or nature of, stage, progression of, and/or grade of the disease in a sample from a subject. The presence, type, stage, and/or grade of the disease may be referred to as the “characteristics” of a disease.

A used herein, the term “detecting” means finding, discovering, identifying, or otherwise becoming aware of an entity that was not previously obvious or clear.

The term “disease” as used herein means a disorder, defect, or dysfunction of structure or function in a subject that produces symptoms and signs known to be associated with that disease. It may also be considered to mean an abnormal physiological state, illness, or sickness. It may include cancers, kidney diseases, hyperthyroidism, or other diseases. Many diseases are defined by the World Health Organisation (WHO) in its International Classification of Diseases.

As used herein, the term “Cancer” may include one or more of breast, ovary, colon, central nervous system, kidney, prostate, bladder, colorectal, liver, lung (non-small cell and small cell), brain, pancreas, stomach, oesophagus, head, or neck cancer or Hodgkins lymphoma, non-Hodgkins lymphoma, chronic lymphocytic leukaemia, acute lymphocytic leukaemia, chronic myeloid leukaemia, or acute myeloid leukaemia, or another cancer.

A “solid cancer” as used herein means a cancer which presents with a tumour.

The phrase “Associated tumour” as used herein means a tumour which presents with a solid cancer.

“Kidney diseases” as used herein may include one or more of dialysis-dependent chronic kidney disease, nondialysis dependent chronic kidney disease, glomerulonephritis, nephropathy, nephritis, renal failure, or other renal disorders.

As used herein, the phrase “type of disease” refers to the specific manifestation of a general disease, characterised by the presentation of particular symptoms and signs in a subject, in particular the location of a defect in structure or function that characterises the general disease. For example, if the general disease is cancer, a type of disease may be breast cancer, colon cancer, or bladder cancer.

As used herein, the phrase “stage of disease” means a point in the progression of a disease which is marked by the disorder, defect, or dysfunction exceeding a particular predetermined threshold. For example, the stages of a cancer, i.e. stage 0, I, II, Ill, and IV.

The phrase “grade of disease” as used herein refers to a qualitative indicator of the aggressiveness of the disease and correlates with prognosis. For example, a tumor may have grading G1, G2, or G3.

As used herein the term “sample” or “biological sample” means a quantity of biological matter that has been removed from an organism, such as a tissue or a fluid. Examples include but are not limited to urine, saliva, whole blood, plasma, platelet rich plasma (PRP), platelet poor plasma (PPP), pooled normal plasma (PNP), and cerebrospinal fluid. Wherein said sample is a fluid, it may also be referred to as a “biological fluid”.

As used herein, the term “subject” means an individual animal. The subject may be any animal, including any vertebrate. The subject may be a domestic livestock, laboratory animal (including but not limited to, rodents such as a rat, hamster, gerbil, or mouse) or pet animal. In one embodiment, the animal may be a mammal. Examples of mammals include humans, primates, marsupials, canines, monkeys, rodents, felines, apes, whales, dolphins, cows, pigs, and horses. The subject may be in need of treatment of a disease or may be in need of a prophylactic treatment.

As used herein, the term “Quantifying” means measuring a physical property of an entity, for example, the amount by weight of the entity contained in a sample.

As used herein, the terms “normal” and “healthy” mean a state of complete physical, mental, and social well-being, though may also be defined as the absence of disease.

As used herein the phrase “Predetermined normal range” means the upper and lower bounds and discrete or continuous values between the upper and lower bounds determined epidemiologically or statistically to represent the values expected from any healthy member of a population.

As used herein the phrase “Screening for a disease” means applying a method for detecting and characterising a disease to samples from multiple subjects (i.e. a control sample, as defined below), and may additionally involve applying one or more statistical analyses to the data obtained to derive further information from it, identify patterns, or otherwise inform on the character of the disease and how the characteristics are distributed between the subjects.

As used herein the term “diagnosis” means to detect the existence of a disease in a sample, or to detect a change or characterising in a sample which is indicative of a disease (for example difference in the sample compared to a normal range as defined above).

As used herein the term “monitoring” refers to observing and checking the progress of a disease over a period of time. The methods of the invention may be used for monitoring the success of a therapy or treatment (as determined by the progress of a disease in a subject determined by using the method of the current invention) and recurrence of tumors.

As used herein, the phrase “control sample” refers to a sample (of biological fluid or tissue, such as blood, urine etc) from an individual, or preferably the pooled results from a population of multiple individuals, with which the sample from the subject to be tested is compared. For example, the level of a TF-STP isoform may be measured in an individual, and compared to the average level of the same isoform in the same biological fluid (such as urine) from a large population of healthy individuals (the control sample). The comparison may also be to a ratio. The control sample may be used to set a “normal range” as defined above. Variance of the result from the individual sample compared to the control or normal range may indicate a disease, stage of a disease, progress of a disease, recurrence of a disease, recovery from a disease or the effectiveness of treatment for example. Preferably, the control sample is taken from multiple healthy individuals. Alternatively the control sample may be taken from a population suffering from a disease, or a particular stage of a disease

ELISAs

As used here, the term “ELISA” is an acronym which stands for Enzyme-Linked Immunosorbent Assay. It is an immunoassay method for detecting and quantifying a target wherein the target is immobilised before contacting with a detection antibody specific for the target, and subsequently introducing a ligand, comprising reporter activity, which binds the antibody. An appropriately designed ELISA can achieve a high degree of specificity and sensitivity for the target.

The term “target” as used herein means a small molecule, macromolecule, or higher-order structure of interest and includes biomarkers.

As used herein the term “biomarker” refers to a molecule, including but not limited to a protein, lipid, polysaccharide, lipopolysaccharide, proteoglycan, lipoproteoglycan, peptide, phosphorylated protein, phosphorylated peptide, small molecule, organic compound, inorganic compound, cell or cellular fragment, organelle or fragment thereof, or combination thereof, which may be detected by standard methods commonly known in the art, the presence, absence, or amount of which in a subject or sample may be indicative of a particular physiological state, such as a disease state.

As used herein the term “phosphorylated” refers to the addition of a phosphate (PO₄³⁻) group to a protein or other organic molecule.

As used herein the phrase “ligand which binds to antibody” may include an anti-immunoglobulin antibody, an anti-immunoglobulin peptide, protein A, protein G, Protein L, or other ligand which binds to immunoglobulin. Alternatively, if the antibody is an antibody conjugate, the ligand may bind to the conjugated moiety. For example, if the conjugated moiety is biotin or poly-biotin, the ligand may be streptavidin. Other binding pairs of conjugated moiety and ligand are envisaged, such as pleckstrin homology domain and inositol, SH3 domain and proline-rich peptides, ubiquitin and ubiquitin binding domain, glutathione-S-transferase and glutathione, maltose-binding protein and maltose, complementary single-stranded molecules of DNA and/or RNA, and other pairs of molecules which bind to one another.

As used herein the phrase “Conjugated antibody” or “antibody conjugate” means an antibody that has been covalently linked to another molecular species, the “conjugated moiety”, such as a lipid, protein, small molecule, or other molecular species. This molecular species may be a label or part thereof, as defined below, or may itself be linked to a label.

As used herein, a “label” or “detectable label” is a marker, such as a molecule, which can be detected, either directly or indirectly. For example, antibodies may be labelled with biotin, which is then detected through its binding to streptavidin labelled horseradish peroxidase. A label may also be referred to as a “reporter”. A label may be detected by physical or chemical methods. Labels may include fluorescent labels or therapeutic agents, encapsulating agents or radioactive moieties for example. A labelled antibody may be referred to as a “labelled detection antibody” or simply a “detection antibody”.

A label may be detected by a detection means. The term “detection means” refers to any means of detecting a label, visually, chemically or otherwise.

The term “capture reagent” where used herein means a reagent which can bind to any component of a sample. Preferably, said capture regent is an antibody, most preferably an antibody specific for TF-STP or a TF-STP isoform (which may be referred to as a “target isoform”—see below). In a preferred embodiment said capture reagent is immobilised, preferably to a solid support. Examples of solid supports include membranes, plates, microwells or beads. When bound, said capture reagent forms a complex with the component it is bound to. An antigen bound to a capture reagent, or a capture reagent-antigen complex, may be referred to as “captured”.

Kits

The term “kit” as used herein refers to any combination of items for use for a specific purpose, for example for use in the methods of the invention disclosed herein. Examples are given above.

Kits containing items in order to achieve the methods of the current invention are described. Preferably said kits may be used to complete the ELISAs described herein.

Preferably said kits comprise at least a first antibody, antibody conjugate, or functional fragment thereof specific for a phosphorylated or unphosphorylated TF-STP isoform, and a ligand that binds to the antibody, antibody conjugate, or functional fragment thereof, wherein the ligand comprises a label.

Preferably said kits further comprise a second antibody, antibody conjugate, or functional fragment thereof, wherein the second antibody, antibody conjugate, or functional fragment thereof is specific for a different epitope on the same TF-STP isoform for which the first antibody, antibody conjugate, or functional fragment thereof is specific.

The kits of the present invention preferably comprise an antibody comprising SEQ ID NO:12 and/or SEQ ID NO:11 or a functional fragment thereof.

Said kits of the current invention may be used to characterise a disease in an individual or population, wherein characterising said disease includes detecting the presence, type or nature of, stage, and/or grade of the disease, or progression of said disease. Preferably said disease is cancer.

The term “isoform” as used herein refers to any of two or more functionally similar proteins that have a similar but not identical amino acid sequence. An isoform may also refer of two or more functionally similar proteins that and identical amino acid sequence where in one or more residues has been modified chemically, for example by phosphorylation. All of SEQ ID NOs:4,5,6,7,8 and 14 are isoforms of TF STP, and all of these isoforms can be found in urine (uTF-STP). An isoform to be identified or quantified by the methods disclosed herein may be referred to as a “target isoform”, to distinguish the isoform to be identified/quantified from the other isoforms which may be present in the tested sample.

### EXAMPLES

### Example 1

### Synthesis of Peptides

Peptides corresponding to amino acid sequences in the c-terminal region of human tissue factor were made commercially by Fusion Technologies (Ireland) using standard solid phase peptide synthesis techniques. Several of the peptides were synthesized with a cysteine residue on the N-Terminal. The cysteine residue was used to conjugate the peptide to keyhole limpet hemocyananine (KLH) protein modified with maleimide functional groups via formation of thioether linkages. The peptide-KLH conjugate was used to immunize rabbits to make polyclonal antibodies to the respective peptide conjugated and to immunize mice to make monoclonal antibodies to said peptide.

The TF-STP peptides synthesized to make polyclonal and monoclonal antibodies were:

1) aa255-263 TF1: NH2-CKENSPLNVS-COOH (containing
SEQ ID NO: 1: CKENSPLNV)
2) aa245-254 TF2: NH2-CRKAGVGQSW-COOH (containing
SEQ ID NO: 2: CRKAGVGQSW)
3) aa245-263 TF3: NH2-CRKAGVGQSWKENSPLNVS-COOH
(containing SEQ ID NO: 3: CRKAGVGQSWKENSPLNVS)
4) aa255-263 TF1-Pser258 NH2-CKEN(pS)PLNVS-COOH
(containing SEQ ID NO: 4: CKEN(pS)PLNVS)
5) aa245- 254 TF2-Pser253 NH2-CRKAGVGQ(pS)W-COOH
(containing SEQ ID NO: 5: CRKAGVGQ(pS)W)

(Notes: NH2 denotes n-terminal; COOH denotes the c-terminal; pS denotes phosphoserine; aaxxx-aaxxx denotes the amino acids corresponding to sequences in full length uTF, which is identicial to TF found in other biological samples)

Because there are no natural full length phosphorylated isoforms of TF commercially available, synthetic phosphoserine peptides corresponding to the STP region of TF were synthesized and used as antigen standards in certain ELISAs. The synthetic peptides are as indicated.

6) aa245-263 TF3-Pser253: NH2-CRKAGVGQ-(pS)
WKENSPLNVS-COOH (containing SEQ ID NO: 6:
CRKAGVGQ-(pS)WKENSPLNVS)
7) aa245-263 TF3-Pser258: NH2-CRKAGVGQSWKEN(pS)
PLNVS-COOH (containing SEQ ID NO: 7:
CRKAGVGQSWKEN(pS)PLNVS)
8) aa245-263 TF3-Pser253/258: NH2-CRKAGVGQ(pS)WKEN
(pS)PLNVS-COOH (containing SEQ ID NO: 8: CRKAGVGQ
(pS)WKEN(pS)PLNVS)

### Example 2

### Preparation of Anti-TF1, Anti-TF2 and Anti-TF3 Polyclonal Antibodies

TF1, TF2 and TF3 synthetic peptides (see SEQ ID NOs:1-3 above) were linked through the N-terminal cysteine residues to keyhole limpet hemocyanin (KLH) with bifunctional thioether linker (Pierce Chemicals) yielding KLH-TF1,KLH-TF2 and KLH-TF3 conjugates. The KLH-conjugates (1 mg) were emulsified with 1 mL Fruends complete adjuvant and 1 mL saline and injected subcutaneously into rabbits. The rabbits was then boosted once a month with the respective KLH-conjugates and bled every 2 weeks after the initial injection. High titer serum usually started after 2-3 months.

Titer of the anti-TF1, anti-TF2 and anti-TF3 antibodies in the serum was measured by serially diluting the serum in PBS to form a set of diluted serum samples. Each of the diluted serum samples (100 μL) was then incubated in individual wells of the high affinity capture plate of containing the bound KLH-peptide conjugate or the unconjugated peptide for 1 hour followed by washing (3 times) each sample well with wash solution. Horseradish peroxidase (HRP) labelled goat anti-rabbit immunoglobulin G (IgG) diluted 1/5000 in 0.5 M potassium phosphate buffer containing 1% BSA, pH 7.8 (100 μL) was added to each sample well and incubated at room temperature for 1 hr. The plate was then washed (3 times) and high kinetic tetramethylbenzidine (HKTMB, 100 μL) (Moss) was added into each well, and the plate was incubated at room temperature for 10 min. The absorbance of the wells were measured on a titer plate reader (Molecular Devices Spectra Max Plus) at 650 nm and recorded for subsequent calculation see below. High titer serum from individual rabbits was pooled separately to form individual lot of serum. Peptide-specific IgG were purified from the serum by immuno-affinity chromatography where the serum was passed through a column of the respective peptides bound to a solid matrix. The bound antibody was eluted with pH 4.0 buffer. Specific antibodies were dialyzed against a phosphate buffer pH 7.4 and the antibodies were stored frozen.

### Example 3

### Generation Monoclonal Antibodies to Synthetic Peptides TF1, TF2, TF1-Pser²⁵⁸ and TF2-Pser²⁵³

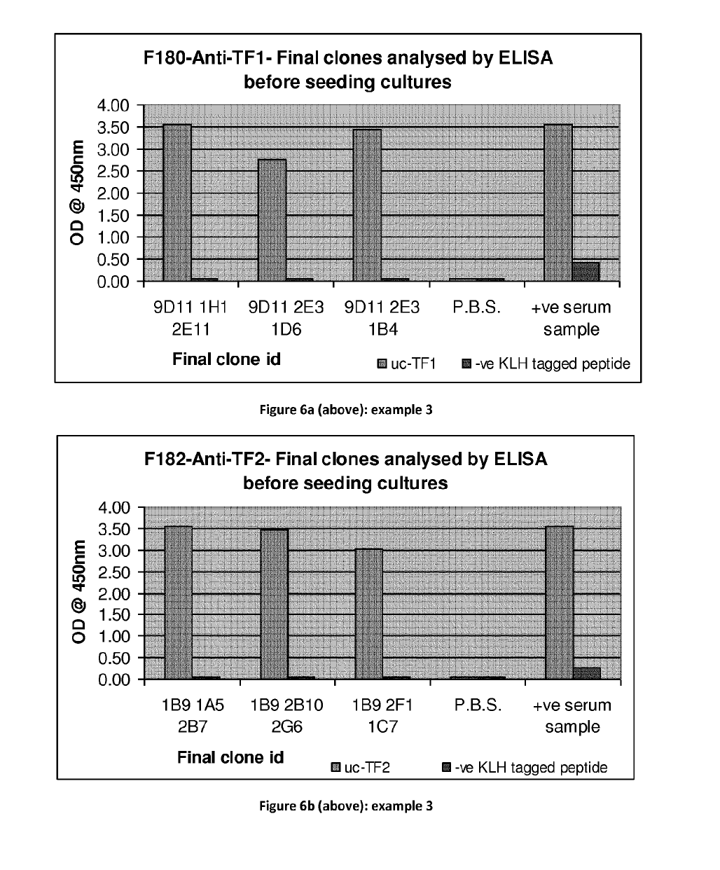

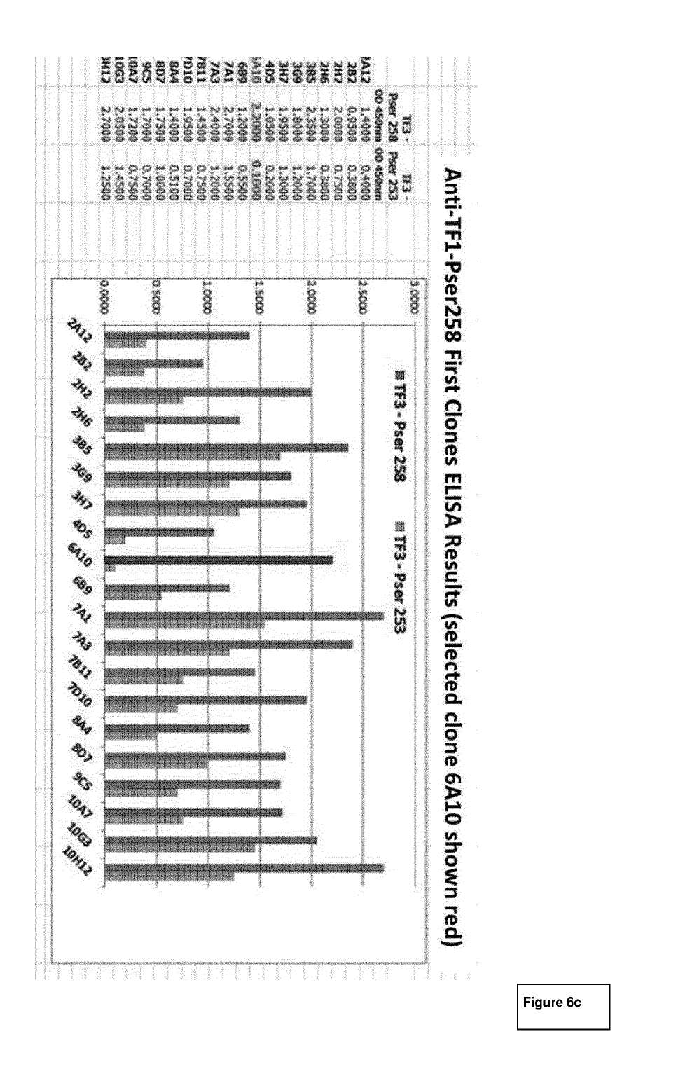

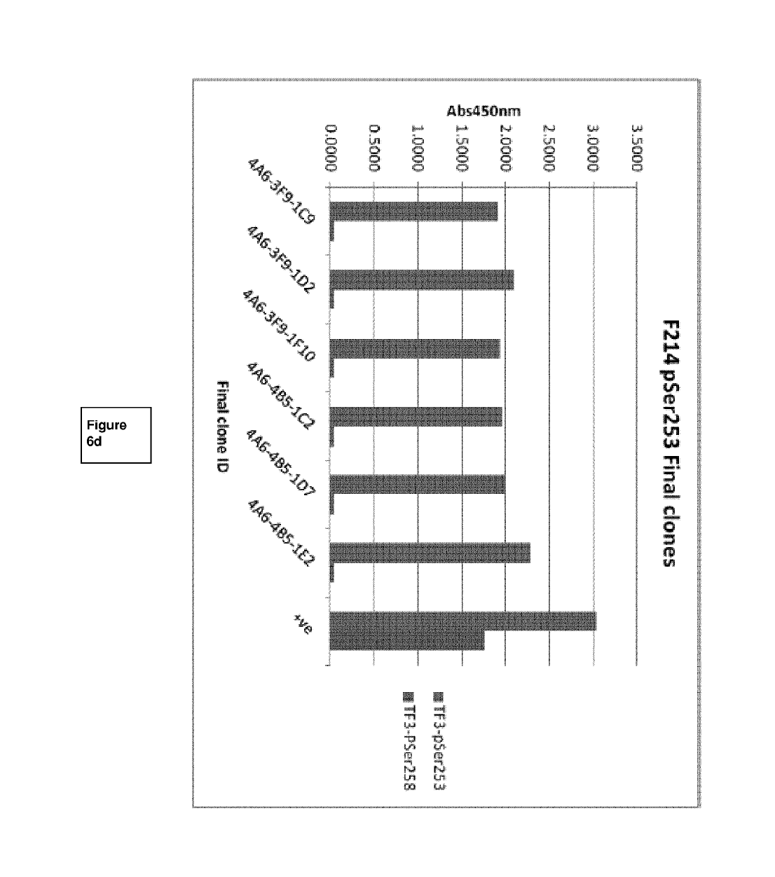

The KLH-TF1, KLH-TF2, KLH-TF1PSer258 and KLH-TF2PSer253 conjugates were made similar to the methods described above for unphosphorylated KLH-peptide conjugates and used to immunize mice for development of hybridomas using standard techniques and protocols known in the art. Hybridomas producing monoclonal antibodies were screened and selected to specifically react preferentially with the respective immunizing peptide (FIG. 6c). Hybridomas were expanded and grown up in vitro in culture medium. Two anti-TF1 MAb clones were selected and expanded 9D11 1H1 2E11 and 9D11 2E3 1B4 (FIG. 6a). Two anti-TF2 MAb clones were selected and expanded 1B9 1A5 2B7 and 1B9 2B10 2G6 (FIG. 6b). Two anti-TF1-Pser²⁵⁸ clones were selected for expansion and designated as MAb clone 6A10 and MAb clone 4D5. One anti-TF2-Pser²⁵³ clone was selected for expansion and designated as MAb clone 4A6-4B5-1E2 (FIG. 6d)). Monoclonal antibodies produced by the hybridomas were purified from the culture medium by affinity chromatography on Protein G columns. The TF1, TF2 and TF1-pser258 MAbs were of the IgG isotype. The TF2-pser²⁵³ MAb was of the IgM isotype with a pentameric structure of MW 750,000 D. Clone nomenclature refers to the microtiter wells the clones were selected from after initial fusion through successive sub cloning.

### Example 4

### Generation of a Genetically Engineered Hybrid Monoclonal Antibody MAb 680 Specific for TF-STP-Pser²⁵³

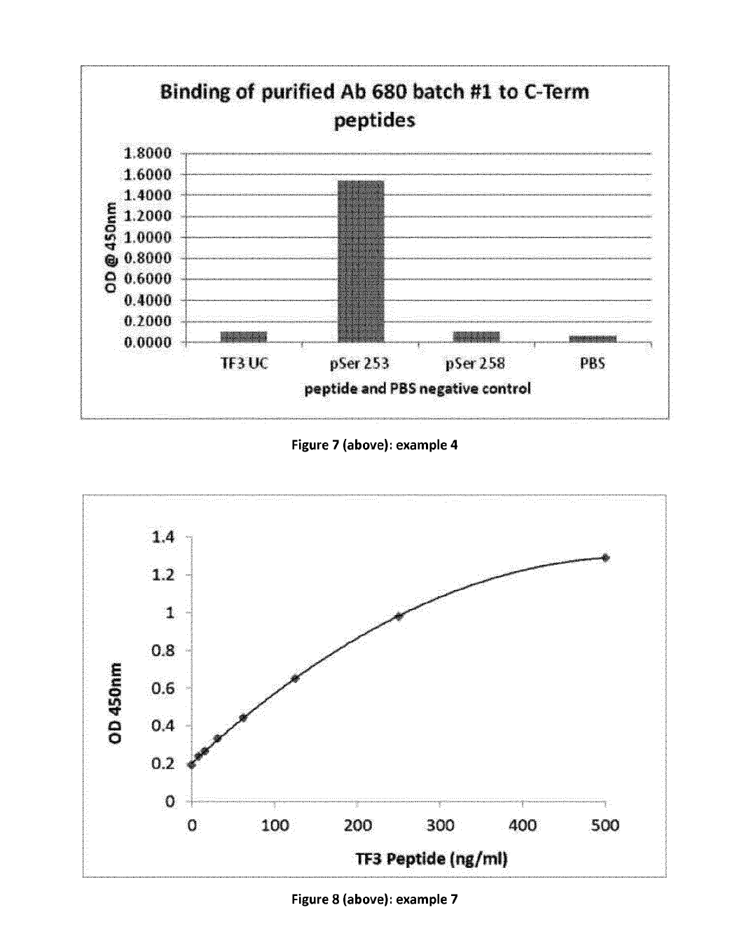

The Fab portion of the light and heavy chain of the anti-TF-Pser²⁵³ IgM monoclonal antibody 46B-4B5-1E2 (MW 750,000 D) was fused onto murine IgG2b light and heavy chains, respectively using genetic engineering techniques to create a hybrid monoclonal antibody of the IgG2b isotype. The hybrid IgGb2 MAb (MW 150,000 D) contains only two binding sites. The hybrid antibody MAb 680 specifically binds to the TF2-Pser²⁵³ peptide but not TF1-Pser²⁵⁸ nor unphosphorylated TF3 peptide (FIG. 7).

### Example 5

### Biotinylation of Antibodies

Secondary antibodies in the two site ELISAs were labelled with biotin using standard techniques. Anti-TF3 Pab IgG, anti-TF1 Pab, IgG and anti-TF3-Pser²⁵⁸ Pab IgG (#ab 62251, Abcam, MA),anti-TF2-Pser²⁵³ hybrid MAb 680 were labelled with biotin according to manufacturer's instructions. Briefly, antibody protein (0.5-5 mgs) is mixed with nhs-LC-LC-Biotin (Pierce Biosciences, IN.) at a biotin: protein ratio of 20:1. After the reaction is complete the proteins are typically dialyzed or passed through a separation column to remove Antibody-biotin conjugate from unbound biotin. The purified conjugated antibodies are stored typically at 4° C.-−80° C. Other anti-TF-STP antibodies described can be labelled with biotin in similar fashion.

### Example 6

### Formation of High Affinity ELISA Capture Plates

The capture antibodies in the two site ELISAs were bound to the plastic ELISAs plates using standard techniques. Antibodies were dissolved in a coating buffer (0.05 M carbonate buffer, pH 9.6) to give a coating solution with concentrations between 1 and 10 μg/mL depending on the antibody. The coating solution (100 μL) was pipetted into each well of Immulon 96-well High Binding 4HBX 96-well microtiter plates and incubated at room temperature for 1 h followed by washing the wells (3 times) with wash solution 200-300 μL PBS containing 0.05% Tween 20, pH 7.8). The wells were then blocked by adding SuperBlock (Pierce Chemicals) and incubated at room temperature for 1 hr. The wells of the microtiter plate were washed several times with the wash solution 200-300 μL and air dried to form the high affinity capture plate.

### Example 7

### ELISA for TF-STP Using Polyclonal Anti-TF1 Antibody as Capture and Anti-TF3 Pab-Biotin for Detection

The ELISA was performed essentially as described in example 5 except the coating antibody was anti-TF1 Pab (0.3 and 0.5 ug/ml) and the detection antibody was anti-TF3 Pab-biotin. The standard was TF3 peptide was added to the elisa plate at various concentrations. A standard curve that fit a polynomial curve fitting program is produced (FIG. 8).

### Example 8

### ELISA for TF-STP Using a MAb for Capture and Polyclonal Antibody as Detection

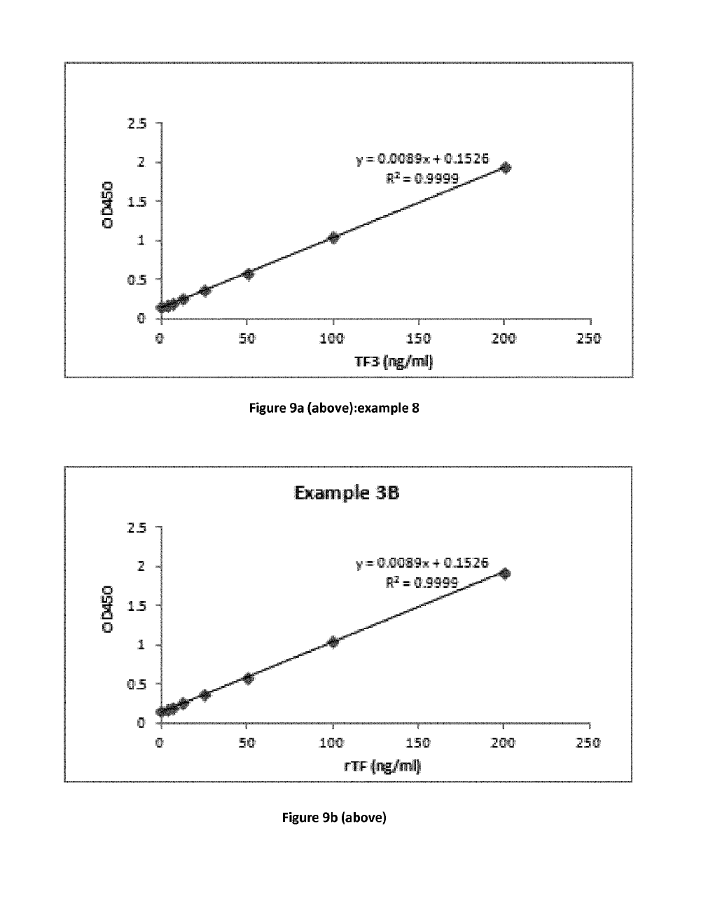

A TF-STP ELISA format using anti-TF2 MAb as the capture antibody and anti-TF3 Pab-biotin as the detection antibody is described in the context of the generation of a standard curve using purified TF3 synthetic peptide as the standard. Anti-TF2 MAb (typically 0.5-5 ug/ml) was bound to Immulon 96-well High Binding 4HBX 96-well microtiter plates at pH 9.6 in carbonate buffer. The TF3 or recombinant TF (rTF) standards were dissolved in 50 mM Tris HCL/0.15 M NaCl/PEG8000/1 mM dithiothreitol/CHAPS 4 mM pH 8.0 sample buffer (CHAPS-Buffer) and serially diluted in sample CHAPS-buffer to give the concentrations ranging from 0 to 100 ug/ml. The standard curve was prepared by incubating 100 μL of the standards in a microtiter plate coated with 1 ug/ml anti-TF2 Pab. After incubating for 16 hours, each of the samples wells were washed (3 times) with wash solution. Anti-TF3 Pab-biotin in 0.5 M potassium phosphate buffer containing 1% BSA, pH 7.8 (100 pL) was added to each sample well and incubated at room temperature for 1.5 hr. The plate was then washed (3 times) and SA-polyHRP (Pierce Chemicals) was added (1:40,000) and incubated for 1.5 hours. The plates were washed three times and high kinetic tetramethylbenzidine (HKTMB, Moss100 μL) substrate was added into each well, and the plate was incubated at room temperature for 10 min. The stop solution 1 N sulphuric acid was added and the absorbance of the wells were measured on a microtiter plate reader (Molecular Devices Spectra Max Plus) at 450 nm and recorded. The recorded absorbance data were correlated with the TF3 peptide (FIG. 9a) or rTF protein (FIG. 9b) standard concentration to form a standard curve, which is a plot of the absorbance as a function of the standard concentration. In this example, all samples were obtained from urine.

### Example 9

### Sample Buffer Effects the ELISA Using Anti-TF2 MAb as Capture Antibody and Anti-TF3 Pab-Biotin as the Detection Antibody

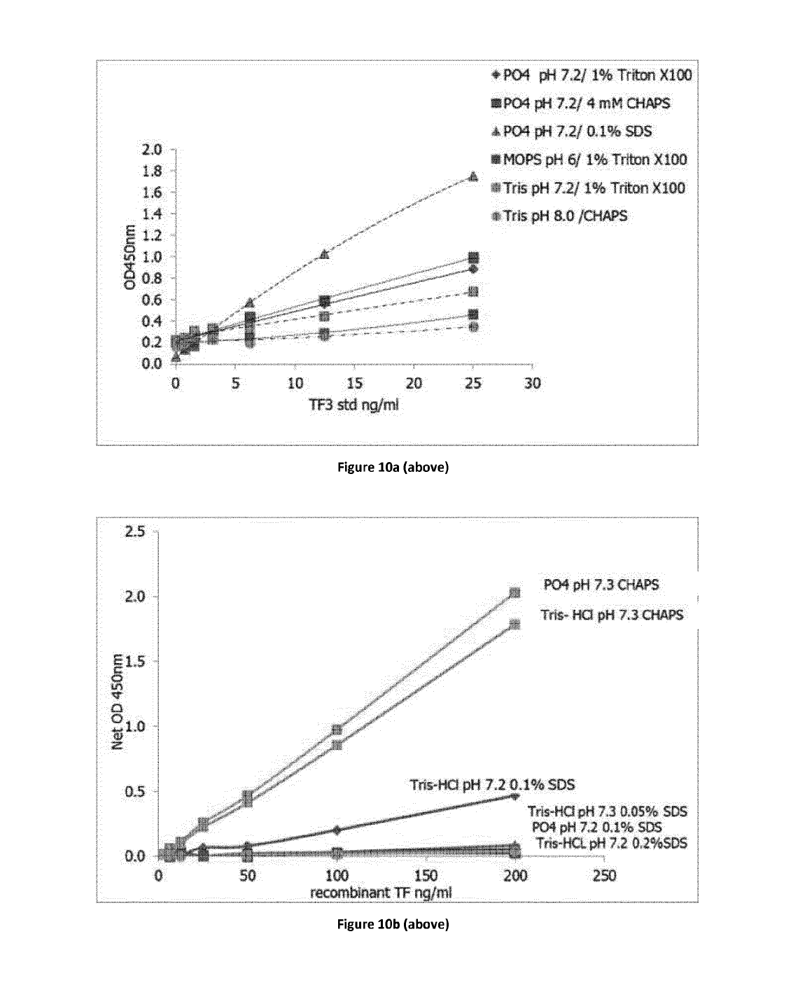

The ELISA was performed essentially as in example 5 except the plate was coated with anti-TF2 MAb (1 ug/ml) and the detection antibody was anti-TF3-Pab-biotin. The Standard TF3 peptide was dissolved in six different buffer compositions containing different detergents, detergent concentrations and different pHs. The low pH buffers at pH 6.0 and 7.3 produced steeper standard curve with TF3 standard than the buffer at pH 8.0. The buffer with SDS ionic detergent produced a steeper standard curve than buffers containing non-ionic detergents CHAPS or Triton X100 (FIG. 10a).

### Example 9 A

### Effect of Buffer Conditions on ELISA Using TF3 Peptide as Antigen

When rTF was used as the standard the PO4/CHAPS or TRIS/CHAPS containing buffers produced steeper standard curve than the SDS containing buffers. The rTF standard produced a steeper standard curve in sample buffer with CHAPS buffer as compared to the TF3 peptide standard (FIG. 10b).

### Example 10

### ELISA Specific for Unphosphorylated TF-STP Isoform

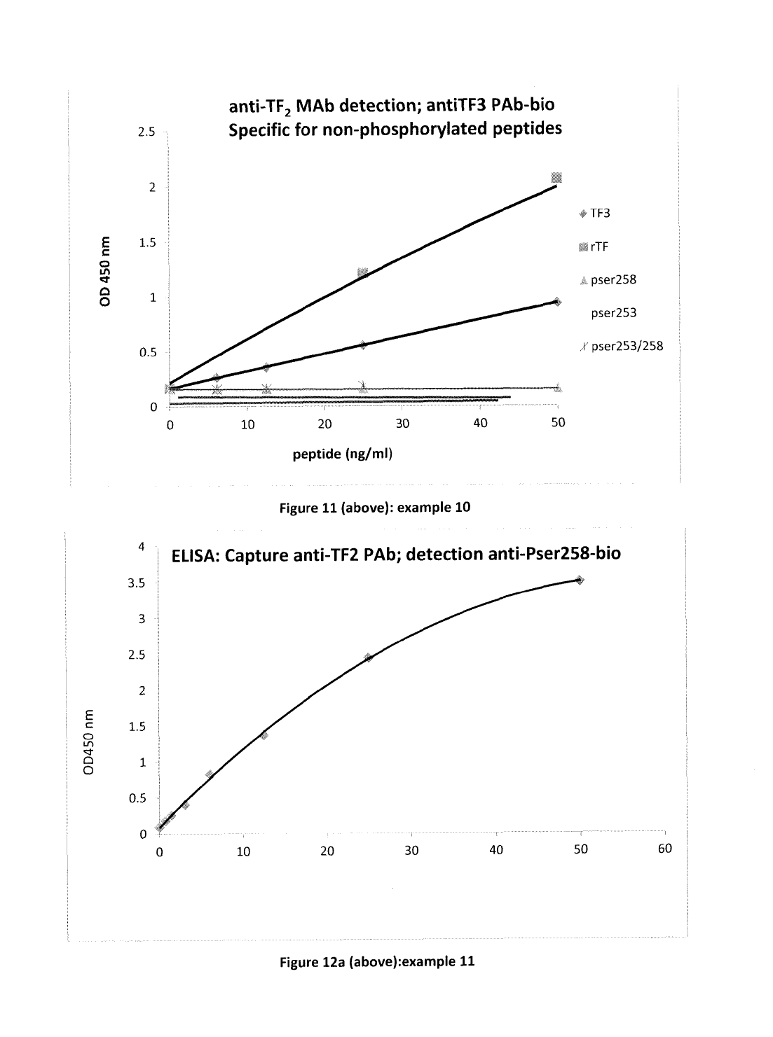

An ELISA that recognizes only unphosphorylated TF-STP antigens was produced using the anti-TF2 MAb as the capture antibody and coated onto the ELISA plate. The anti-TF3 Pab-biotin is used as the detection antibody. The ELISA was carried out similar to Example 5 above. The sample buffer was 20 mM sodium phosphate, 0.15 M NaCl, 3% PEG8000 and 4 mM CHAPS pH 7.4. The ELISA specifically recognizes unphosphorylated TF3 peptide and unphosphorylated recombinant Tissue factor (rTF) but not phosphorylated peptides TF3-pser253, TF3-pser258 nor diphosphorylated TF3-pser253/258 peptide is shown below (FIG. 11). The samples used were urine.

### Example 11

### ELISA Specific for TF-Pser²⁵⁸ Isoform

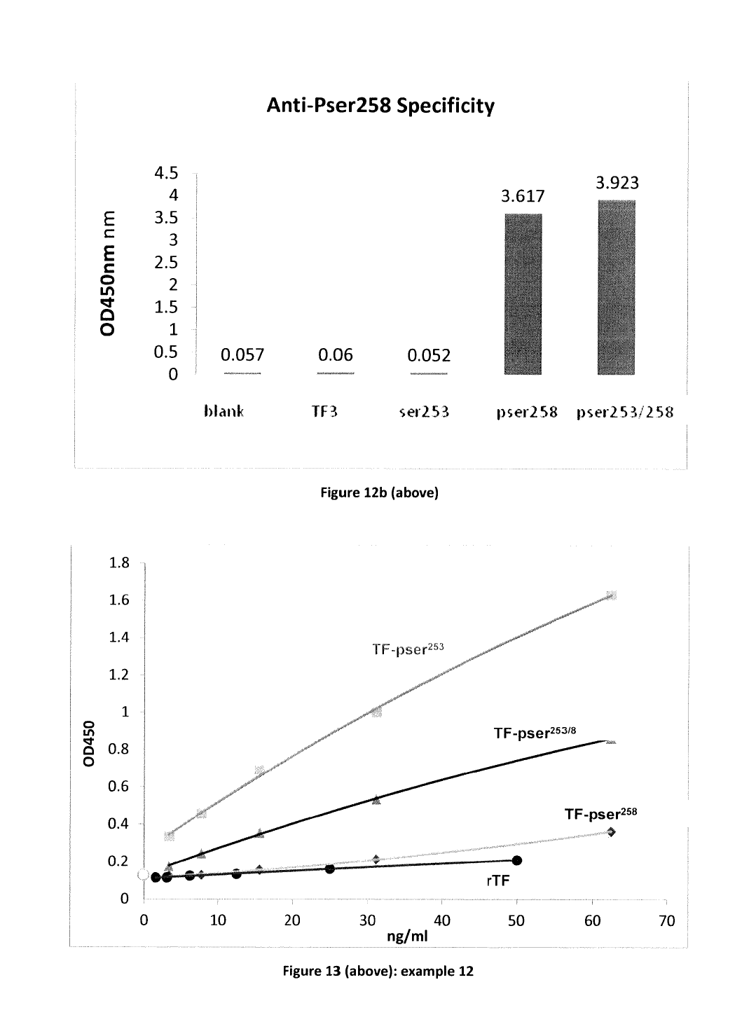

An ELISA specific for TF-STP-PSER258 antigens was produced using anti-TF2 Pab as the capture antibody and coated onto the ELISA plate. The anti-TFpser258 Pab (#ab62251) was biotinylated and used as the detection antibody. The ELISA was carried out using the general conditions stated in example 5. The sample buffer was 50 mM Tris-HCl, 0.15 M NaCl, 3% PEG8000, 0.02% sodium dodecylsulfate at pH 7.3. The detection antibody buffer was 20 mM HEPES, 0.15 M NaCl, 3% PEG8000, 0.5% bovine serum albumin, 0.1% tween 20 pH 7.3. The ELISA produced a standard curve using TF3-Pser258 peptide that fit a polynomial curve (FIG. 12a). The ELISA specifically recognizes the phosphorylated peptides TF3-pser258 and TF3-pser253/258 peptide but not unphosphorylated recombinant Tissue factor (rTF), unphosphorylated TF3 nor phosphorylated peptide TF3-pser253 (FIG. 12b). The samples used were urine.

### Example 12

### ELISA Specific for TF-Pser²⁵³ Isoform

An ELISA specific for TF-STP-Pser253 antigens was produced using of anti-TF2-Pser253 hybrid MAb 680 as the capture antibody and coated onto the ELISA plate. The anti-TF2 Pab-biotin was used as the detection antibody. The ELISA was carried out using the general conditions stated in example 5. The samples used were urine. The sample buffer was 50 mM Tris-HCl, 0.15 M NaCl, 3% PEG8000, 0.02% sodium dodecylsulfate at pH 7.3. The detection antibody buffer was 20 mM HEPES, 0.15 M NaCl, 3% PEG8000, 0.5% bovine serum albumin, 0.1% Tween 20 pH 7.3. The ELISA produced a standard curve using TF3-Pser253 peptide that fit a polynomial curve. The ELISA specifically recognizes the phosphorylated peptides TF3-pser253 and TF3-pser253/258 peptide but not unphosphorylated recombinant Tissue factor (rTF), unphosphorylated TF3 nor phosphorylated peptide TF3-pser258 (FIG. 13).

An anti-phospho-PKC-substrate motif antibody (Cell Signalling Technology, New England Biolabs, Hitchin, UK) that binds to TF-pser253 did not produce a usable ELISA when used as the capture or detection antibody. Similarly, a usable ELISA was not produced using the pentameric anti-uTF-STP-Pser253 IgM MAb 46B-4B5-1E2.

### Example 13

### Quantitation of Unphosphorylated uTF-STP, uTF-STP-Pser²⁵³ and uTF-STP-PSer²⁵⁸ in Unfractionated Urine Samples from Healthy and Cancer Patients Using ELISA Methods

Unphosphorylated and phosphorylated TF isoforms were measured using ELISAs in unfractionated urine samples obtained from healthy individuals and from patients diagnosed with various cancers. Frozen urine samples from five healthy subjects and five colon cancer, five prostate cancer, five bladder cancer and five breast cancer patients were obtained through commercial sources. Frozen urine samples were thawed and 4 ml of urine was centrifuged at 400×g for 20 minutes to pellet particulates and debris. Unphosphorylated TF isoform was measured by mixing the pre-cleared urine supernatants with equal volumes of 2× sample buffer containing 0.1 mM sodium phosphate, 0.3 M NaCl, 8 mM CHAPS, pH7.3. Samples were measured for unphosphorylated uTF-STP using the ELISA method in example 11.

To quantitate uTF-STD-pser253 and uTF-STD-pser258, the pre-cleared urine supernatants were mixed with equal volumes of 2× sample buffer containing 50 mM Tris-HCL, 0.3 M NaCl, 0.04% SDS, PEG8000 pH 7.3. Samples were measured for uTF-STP-PSER258 isoform using the ELISA in example 12. Samples were measured for uTF-STP-Pser253 isoform using the ELISA method in example 13. The levels of each isoform in the urine sample were determined by interpolating the OD450 nm using the respective standard curves generated in each ELISA. The results of the quantitation of uTF-STP, uTF-STP-Pser253 and uTF-STP-Pser258 in urine fractions from healthy and cancer patients are listed in Table 1 below.

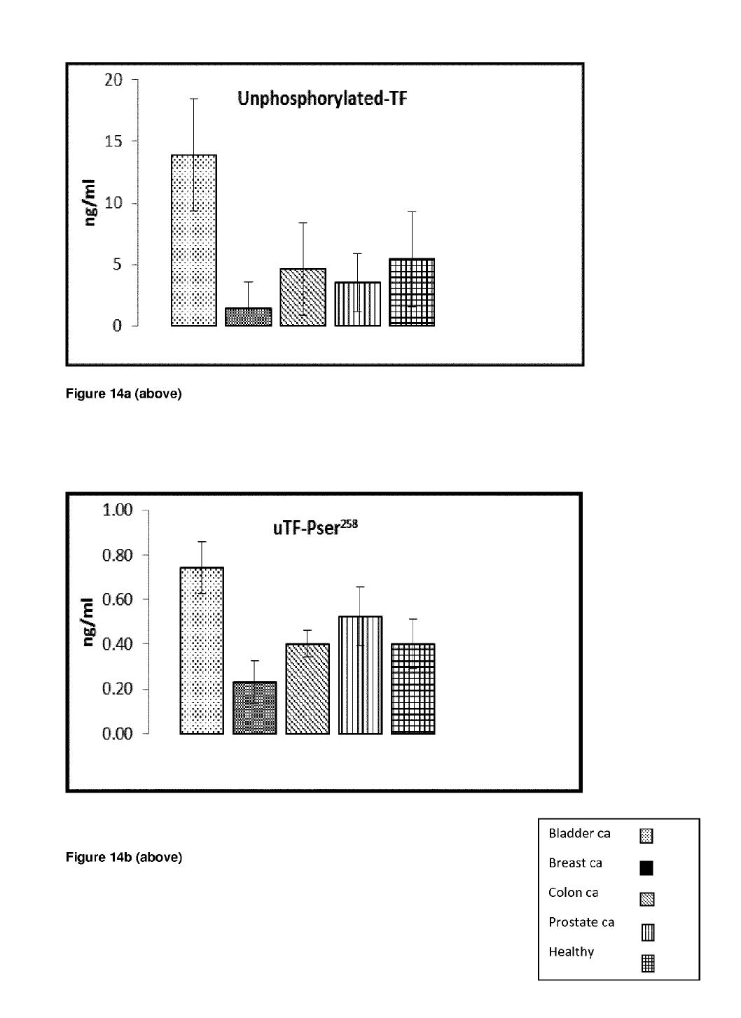

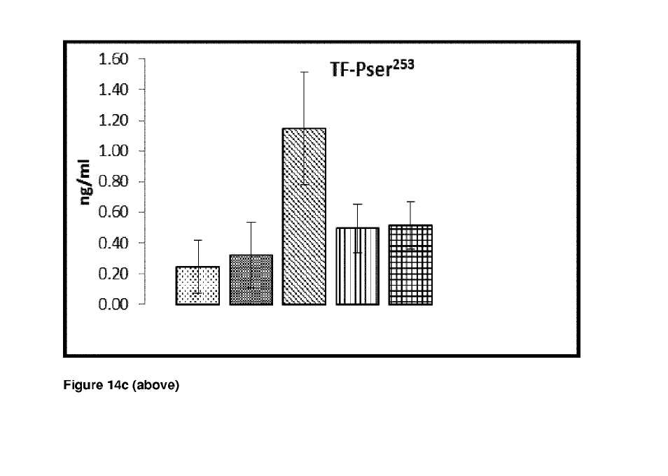

The tabular results are shown in pictorial graphic in the Figures discussed below. The results show that the each of the three TF isoforms can be detected and quantitated in human urine samples using the ELISA methods as described. The unphosphorylated TF (FIG. 14a) and uTF-STP-Pser²⁵⁸ (FIG. 14b) isoforms are present in higher levels in bladder cancer compared to healthy as well as prostate, colon and breast cancer samples. The uTF-STP-pser²⁵⁸ in the prostate cancer group is also higher compared to the healthy group. The uTF-STP-pser253 isoform is higher in the colon cancer group compared to the healthy and prostate, bladder and breast cancer groups (FIG. 14c). The quantitation of uTF isoforms appears to be useful for detection and diagnosis of various cancers diagnosis.

### Example 14

### Quantitation of uTF-STP in Fractionated Urine from Normal and Cancer Patients

uTF-STP was measured in microparticles present in human urine. The urine samples were centrifuged at 14,000×g to pellet the microparticle fraction. Frozen urine samples from 50 normal urine samples and 95 urine samples from colon, prostate breast and bladder cancer patients were obtained from commercial sources. Frozen samples were thawed and 4.5 ml of urine was spun at 400×g to remove debris. The supernatants were centrifuged in a bench top high speed centrifugation at 14,000×g for 20 minutes. After drawing off the supernatants, the pellets were solubilized in 200 μl of sample buffer containing 0.05 mM Tris-HCL, 0.1 M NaCl, 4 mM CHAPS, 1 mM DTT, pH8.0. The samples were then measured for total uTF-STP in the ELISA described in Example 10. uTF-STP levels were interpolated from the rTF standard curve. The Table 3 shows the quantative results of the ELISA test. The optimal cut-off based on the results from the normal population was determined to be 6 ng/ml. Using this cut-off, 48/50 normal samples were negative. This means there was a 4% false positive rate. Of the cancer 95 cancer samples tested 52% of colon cancers were positive, 40% of prostate cancers were positive, 80% of bladder cancers were positive and 0% of breast cancers were positive. Overall, 41/43 positive samples were cancers which results in a test that has an excellent Positive Predictive Value (PPV) of 95.3% (Table 2).

### Example 15

### Quantitation of uTF-STP in Fractionated Urine from Normal and Cancer Patients Using ELISA

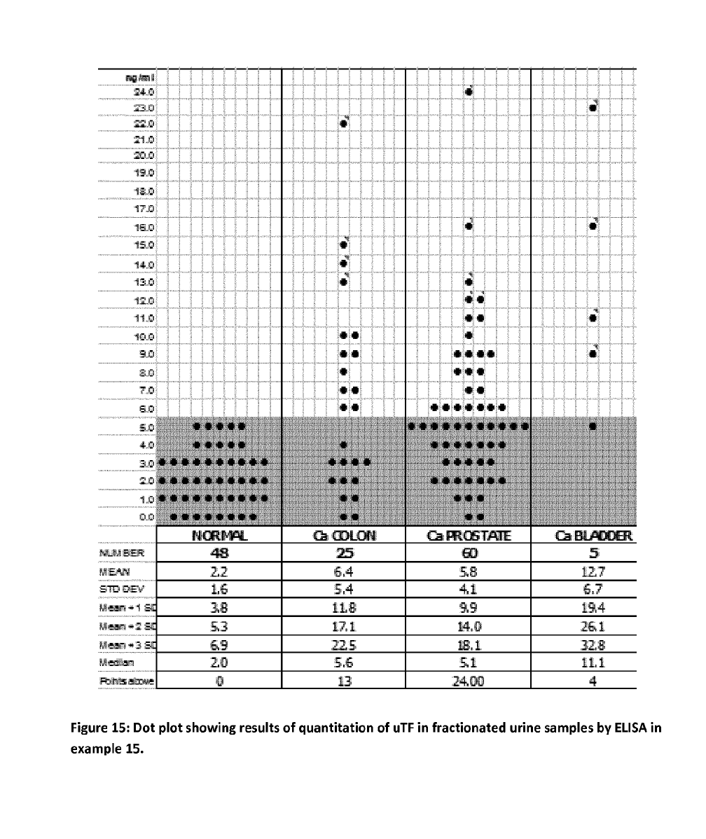

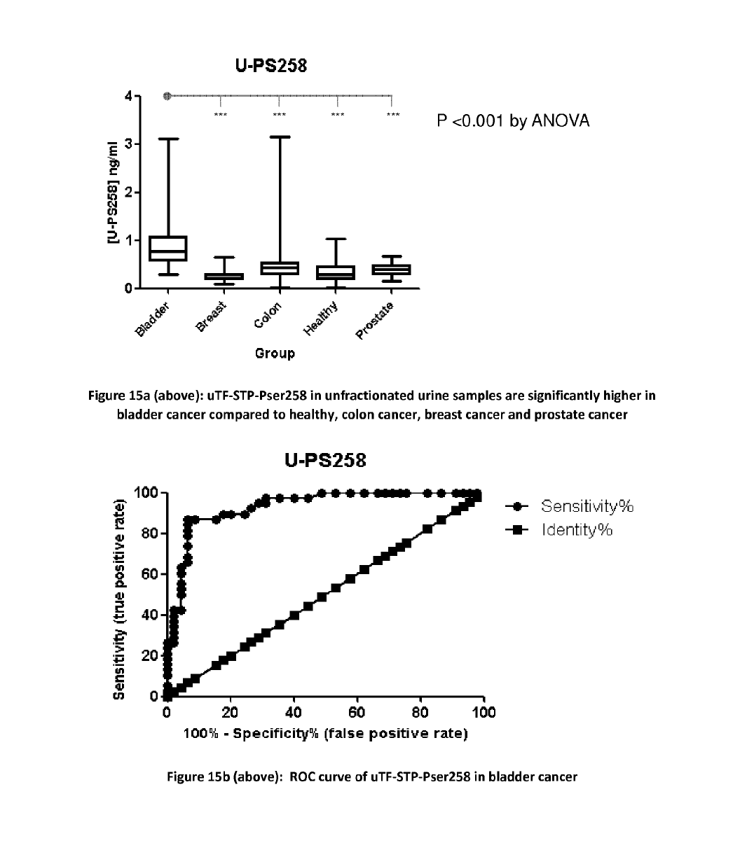

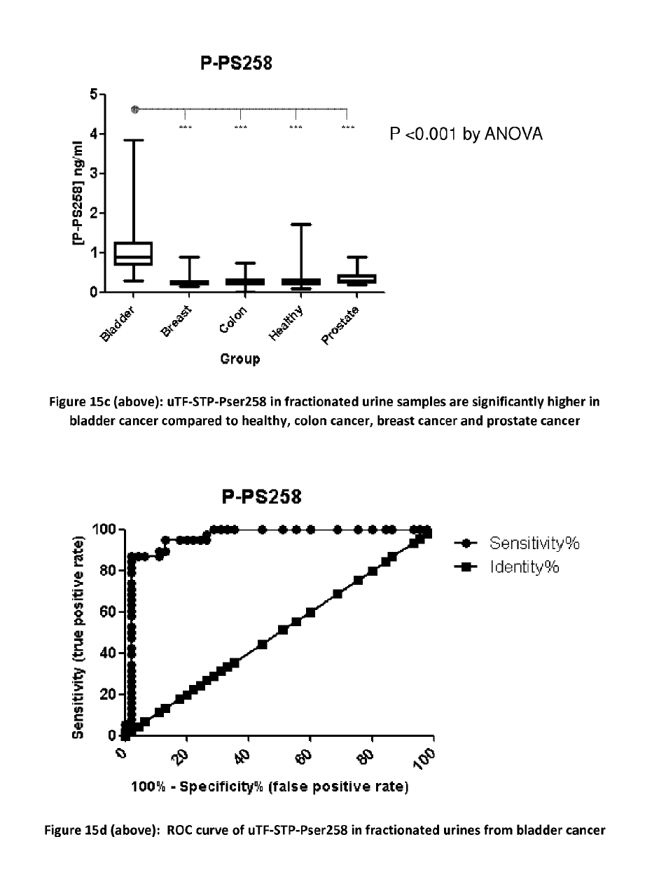

Frozen urine samples were obtained from normal, colon cancer, prostate cancer and bladder cancer through commercial sources. Frozen samples were thawed and 4.5 ml of urine was spun at 400×g to remove pelleted debris. The supernatant was centrifuged in a bench top high speed centrifugation at 14,000×g for 20 minutes. The supernatant was removed and the pelleted microparticles containing uTF-STP was solubilized in a 200 ul of sample buffer 0.05 mM Tris-HCL, 0.1 M NaCl, 4 mM CHAPS, 1 mM DTT, pH8.0. The sample was then applied in the anti-TF2 Mab/anti-TF3Pab-bio elisa. uTF-STP levels were interpolated from the rTF standard curve. FIG. 15 shows the results of the ELISA test. The optimal cut-off based on the results from the normal population was determined to be 6 ng/ml. Using this cut-off, 48/50 normal samples were negative (FIG. 15). Of the 90 cancer samples tested 52% of colon cancers were positive, 40% of prostate cancers were positive, 80% of bladder cancers. Overall, 41/43 positive samples were cancers which results in a test that has an excellent Positive Predictive Value (PPV) of 95.3% (FIG. 15).

### Example 16

### Quantitation of uTF-STP Isoforms in Fractionated and Unfractionated Urine Using an ELISA Specific for Unphosphorylated uTF-STP, an ELISA Specific for uTF-STP-PSer258 and an ELISA Specific for uTF-STP-Pser253

Samples. Frozen urine samples were obtained from normal, colon cancer, prostate cancer, bladder cancer and breast cancer through commercial sources. The urine samples from cancer patients were characterized for grade and stage in addition to cancer type. In this study a 50 normal urine samples and a total of 170 cancer samples were tested—40 bladder cancers, 50 colon cancers, 40 prostate cancers and 40 breast cancers. The cancers were colon, breast, bladder and prostate types.

Sample Preparation. Urine samples were kept frozen in liquid nitrogen or at −80oC until used. Frozen samples were thawed and 4 ml of urine was pre-cleared by centrifugation at 400×g to remove debris. The pre-cleared urine samples were prepared in four samples: two unfractionated samples for testing in the ELISAs and two fractionated samples for testing in the ELISAs. The unfractionated samples were prepared by adding equal volumes of 2× sample buffer 0.1 mM sodium phosphate, 0.3 M NaCl, 8 mM CHAPS, pH7.3 to the pre-cleared sample; and adding equal volumes of 2× sample buffer 50 mM Tris-HCL, 0.3 M NaCl, 0.04% SDS, PEG8000 pH 7.3.

The fractionated samples were prepared by centrifugation of the pre-cleared samples in a bench top high speed centrifugation at 14,000×g for 45 minutes. The supernatant was removed and the pelleted microparticles containing uTF-STP was solubilized in 200 ul of sample buffers comprising 0.05 mM sodium phosphate, 0.0.15 M NaCl, 4 mM CHAPS, pH7.3.; or 50 mM Tris-HCL, 0.15 M NaCl, 0.02% SDS, PEG8000 pH 7.3.

ELISAs. The unfractionated and fractionated sample preparations for each urine sample were then tested in three ELISAs to measure unphosphorylated uTF-STP isoform, uTF-STP-Pser258 isoform and uTF-STP-Pser253 isoform, respectively. The levels of uTF-STP unphosphorylated and uTF-STP-Pser258 were obtained for the unfractionated sample and the fractionated sample by interpolating the OD450 nm using the respective standard curves generated in each ELISA.

ELISA Results. The results of the quantitation of uTF-STP, uTF-STP-Pser253 and uTF-STP-Pser258 in urine fractions from healthy and cancer patients are listed in Table 3 below.

### Example 17

### Analysis uTF-STP-Pser258 levels in Unfractionated and Fractionated Urine from Healthy Individuals and Patients with Cancers

The amount of uTF-STP-Pser258 was measured by ELISA in unfractionated urine samples from healthy individuals and patients bladder, colon, prostate and breast cancers. Using a cut-off of 0.51 ng/ml, uTF-STP-Pser258 was significantly higher in bladder cancers than in colon, breast, prostate and healthy individuals (FIG. 15a). Results from Receiver Operator Curve (ROC) analysis indicated the uTF-STP-Pser258 biomarker had a sensitivity of 86% and specificity of 91% (FIG. 15b).

The amount of uTF-STP-Pser258 was measured by ELISA in fractionated urine samples from healthy individuals and patients bladder, colon, prostate and breast cancers. Using a cut-off of 0.41 ng/ml, uTF-STP-Pser258 was significantly higher in bladder cancers than in colon, breast, prostate and healthy individuals (FIG. 15c). Results from Receiver Operator Curve (ROC) analysis indicated the uTF-STP-Pser258 biomarker had a sensitivity of 89% and specificity of 88% (FIG. 15d).

### Example 18

### Quantitation Unphosphorylated uTF-STP by ELISA in Unfractionated Urine and Fractionated Urine from Healthy Individuals and Patients with Bladder Cancer

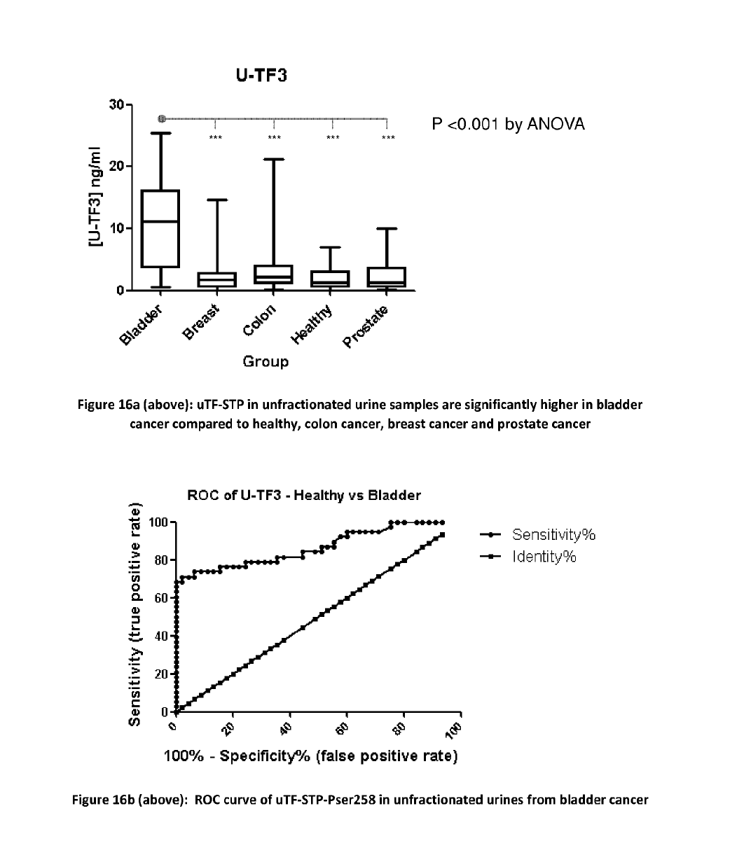

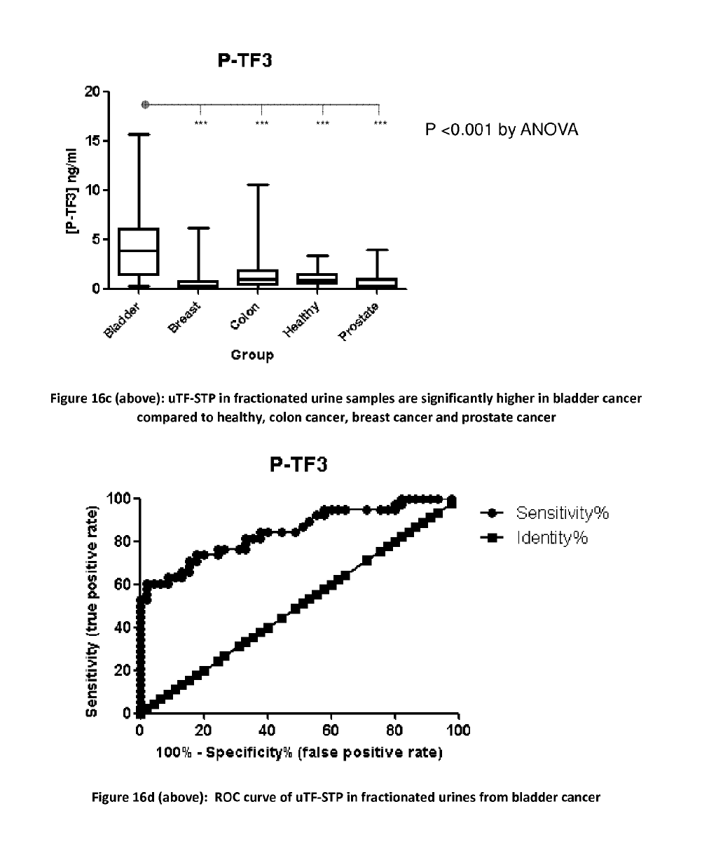

The amount of unphosphorylated uTF-STP was measured by ELISA in unfractionated urine samples from healthy individuals and patients' bladder, colon, prostate and breast cancers. Using a cut-off of 3.5 ng/ml, unphosphorylated uTF-STP was significantly higher in bladder cancers than in colon, breast, prostate and healthy individuals (FIG. 16a). Results from Receiver Operator Curve (ROC) analysis indicated the unphosphorylated uTF-STP biomarker had a sensitivity of 76% and specificity of 77% (FIG. 16b).

The amount of unphosphorylated uTF-STP was measured by ELISA in fractionated urine samples from healthy individuals and patients' bladder, colon, prostate and breast cancers. Using a cut-off of 1.36 ng/ml, unphosphorylated uTF-STP was significantly higher in bladder cancers than in colon, breast, prostate and healthy individuals (FIG. 16c). Results from Receiver Operator Curve (ROC) analysis indicated the unphosphorylated uTF-STP biomarker had a sensitivity of 76% and specificity of 77% (FIG. 16d).

### Example 19

### Quantitation uTF-STP-Pser253 by ELISA in Unfractionated Urine from Healthy Individuals and Patients with Cancer

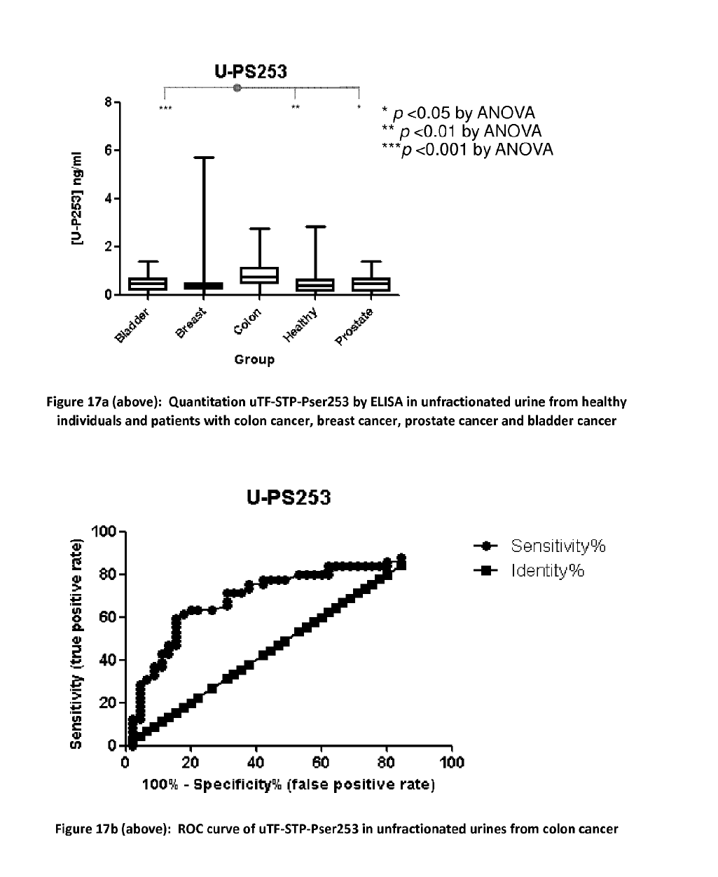

The amount of uTF-STP-Pser253 was measured by ELISA in unfractionated urine samples from healthy individuals and patients' bladder, colon, prostate and breast cancers. Using a cut-off of 0.54 ng/ml, uTF-STP-253 was significantly higher in colon cancers than in bladder, breast, prostate and healthy individuals (FIG. 17a). Results from Receiver Operator Curve (ROC) analysis indicated the uTF-STP-253 biomarker had a sensitivity of 71% and specificity of 69% (FIG. 17b).

According to theory, the synthesis and quantity of the three uTF isoforms are interrelated. The quantitative relationship of any two uTF-isoforms can be of diagnostic value. The ratio of the amounts of each TF-isoform in the fractionated and unfractionated urine samples can be calculated. For example, the individual amounts of each isoform can be quantitated in an ELISA and the resultant ratios of the quantitated values can be calculated. The ratios of TF-isoforms from healthy individuals can be compared to ratios from cancer. If the ratios of cancer samples are significantly higher or lower than in healthy, then there is a likelihood that the ratio is an indicator or biomarker of cancer. Using the quantitative data for individual isoforms in Table 3, the following ratios can be calculated: P-TF3/U-PS258, U-253/U-PS258, UTF3/P-TF3, U-PS253/P-PS253, P-TF3/P258, PTF3/P258. In examples 20-22 certain ratios are significantly different in the cancer groups as compared to the healthy group indicating the calculated ratios of various TF-isoforms are cancer biomarkers.

### Example 20

### Ratio of Unphosphorylated uTF-STP in Unfractionated and Fractionated Urine is a Biomarker for Prostate and Breast Cancer

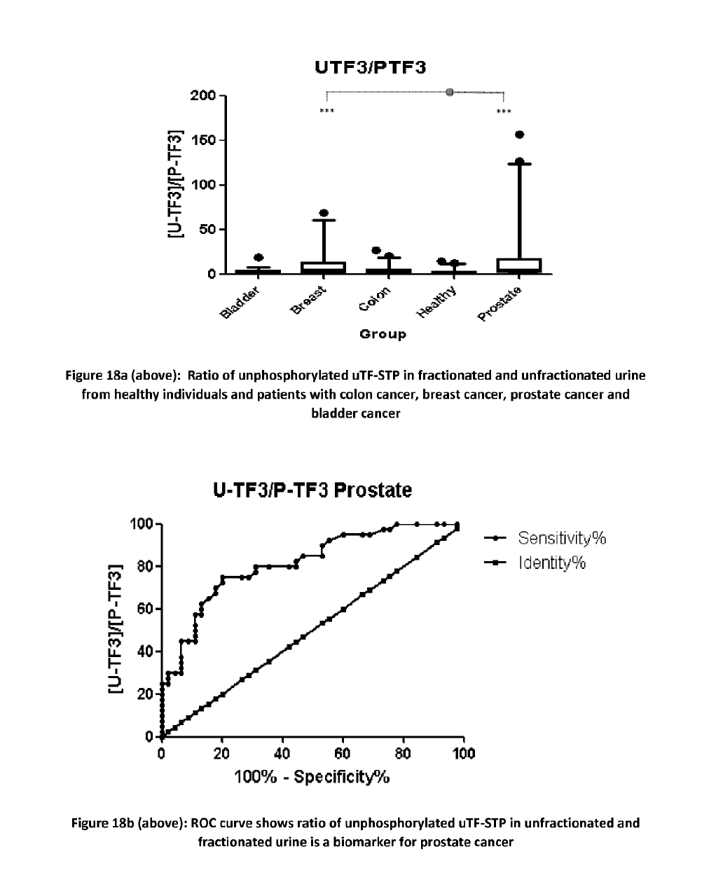

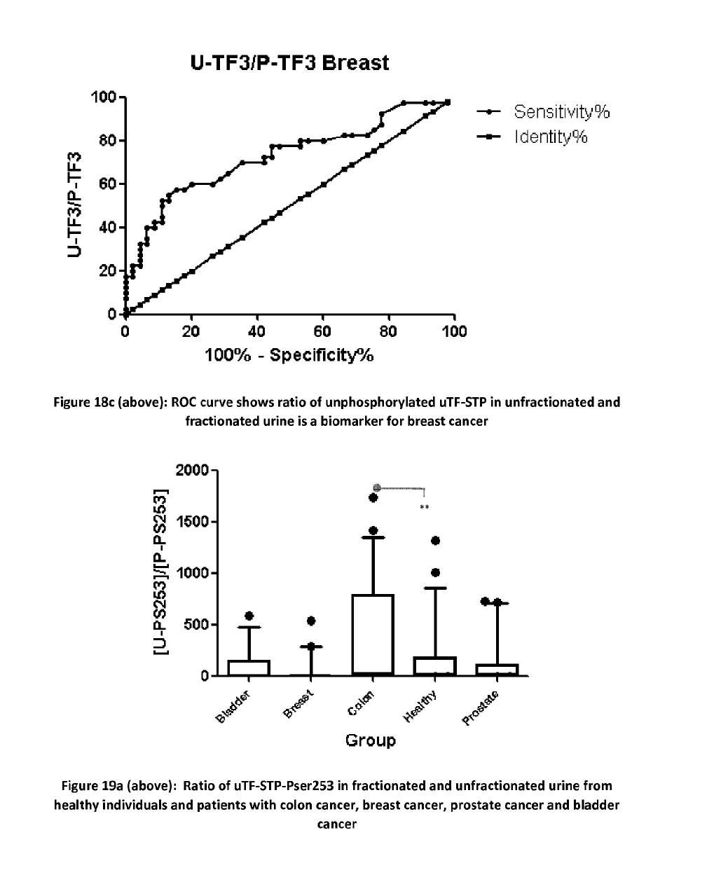

The amount of unphosphorylated uTF-STP was measured by ELISA in unfractionated and fractionated urine samples from healthy individuals and patients bladder, colon, prostate and breast cancers. The ratio of the unfractionated vs fractionated values were calculated. Using a cut-off of 2.95, ratio of unphosphorylated TF was significantly higher in prostate and breast cancers than in bladder, colon and healthy individuals (FIG. 18a).

Results from Receiver Operator Curve (ROC) analysis indicated the ratio had a sensitivity of 75% and specificity of 73% in prostate cancer (FIG. 18b) and 70% and 64% in breast cancer (FIG. 18c).

### Example 21

### Ratio of uTF-STP-253 in Unfractionated and Fractionated Urine is a Biomarker for Colon Cancer

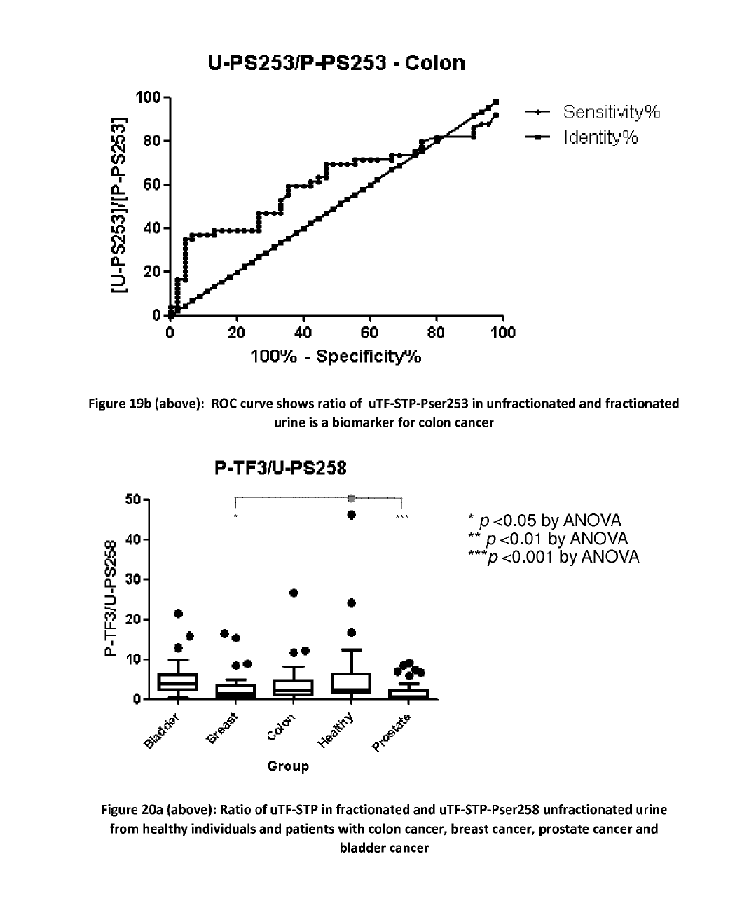

The amount of uTF-STP-Pser253 was measured by ELISA in unfractionated and fractionated urine samples from healthy individuals and patients bladder, colon, prostate and breast cancers. The ratio of the unfractionated vs fractionated values were calculated. Using a cut-off of 10, ratio of uTF-STP-Pser253 in unfractionated and fractionated urine was significantly higher in colon cancer than in bladder, breast, prostate and healthy individuals. Results from Receiver Operator Curve (ROC) analysis indicated the ratio had a sensitivity of 59% and specificity of 51% in colon cancer (FIGS. 19a and 19b).

### Example 22

### Ratio of Unphosphorylated uTF-STP in Fractionated Urine and uTF-STP-Pser258 in Unfractionated Urine is a Biomarker for Prostate and Breast Cancer

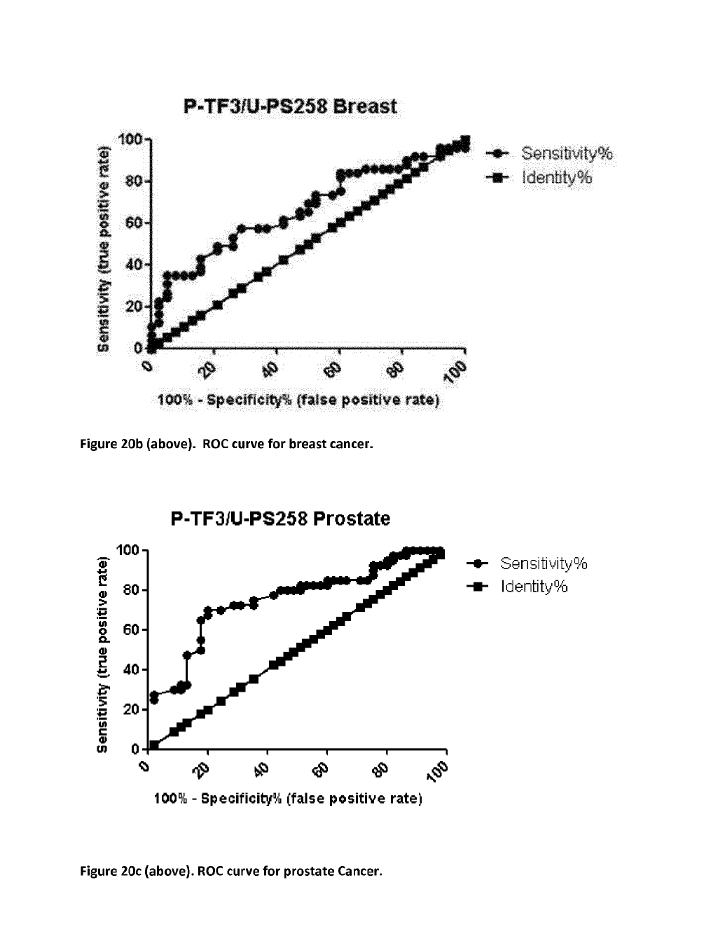

The amount of unphosphorylated uTF-STP and uTF-STP-Pser258 was measured by ELISA in unfractionated and fractionated urine samples from healthy individuals and patients bladder, colon, prostate and breast cancers. The ratio of unphosphorylated uTF-STP in the fractionated urine vs uTF-STP-Pser258 in unfractionated values were calculated. Using a cut-off of 1.75, ratio of in unfractionated and fractionated urine was significantly lower in prostate and breast cancer than in bladder, colon and healthy individuals (FIG. 20a). Results from Receiver Operator Curve (ROC) analysis indicated the ratio had a sensitivity of 67% and specificity of 69% in breast cancer (FIG. 20b) and in prostate cancer, the sensitivity was 72% and specificity was 71% (FIG. 20c).

### Example 23

### uTF-STP-Pser258 in Fractionated and Unfractionated Samples from Different Stage Tumors

uTF-STP-Pser258 levels were measured in unfractionated and fractionated urine samples from various colon, prostate, breast and bladder cancer patients. Stages were determined via analysis by qualified clinical pathologists. uTF-STP-Pser258 levels are inversely proportional to the stage of a tumor (FIG. 21) in both fractionated and unfractionated samples. Higher levels of uTF-STP-Pser258 (0.6 ng/ml) are associated with early stage (0-I) tumors, whereas, lower levels (0.2 ng/ml) is associated with late stage (III-IV) tumors.

### Example 24

### Unphosphorylated uTF-STP is a Biomarker for Staging Bladder Cancers

Bladder cancers of stages I-IV were measured for uTF-STP and uTF-STP-Pser258 levels in fractionated and unfractionated urine samples. In 9 out of 12 (75%) samples of Stage 0 bladder cancers the unphosphorylated uTF-STP was not elevated above healthy cut-off. However, eighty three percent (10/12) of the stage 0 bladder cancers had elevated levels of uTF-STP-Pser258.

Bladder cancers of stages I-IV are characteristically high in both uTF-STP and uTF-STP-Pser258 (Table 4). Thus, bladder cancer stages 0 and I-IV can be discriminated using diagnostic ELISAs which determine the levels of both unphosphorylated uTF-STP and uTF-STP-Pser258.

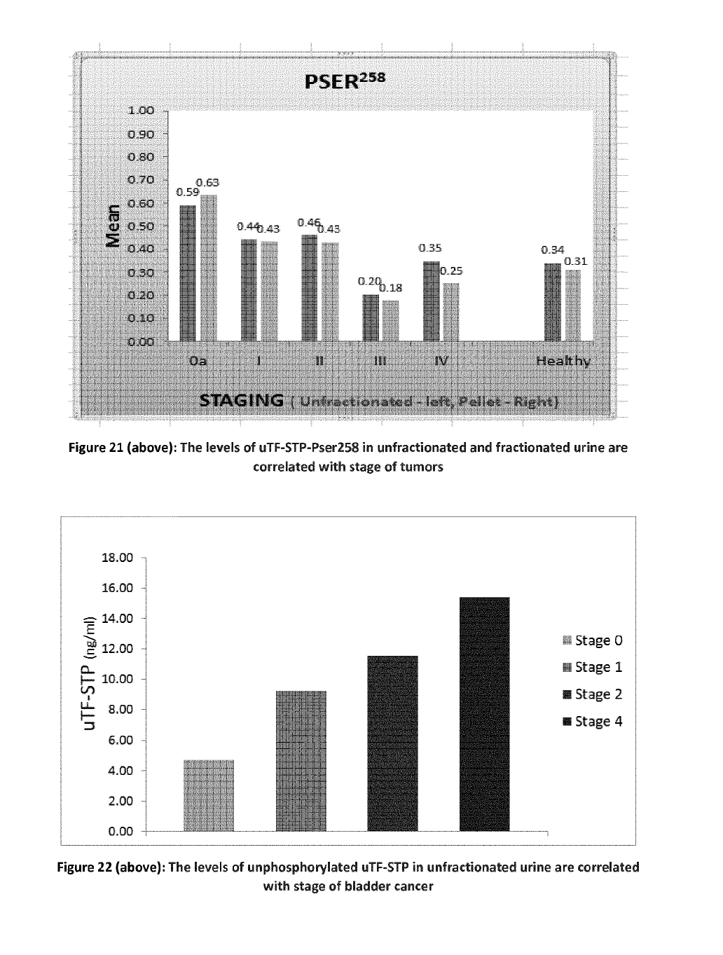

Unphosphorylated uTF-STP levels in unfractionated samples are highly correlated with staging of bladder cancer as increasing amounts of uTF-STP are seen as the stage of bladder cancers increase from I-IV (FIG. 22).

Unphosphorylated uTF-STP and uTF-STP-Pser258 levels are strong biomarker tandem for staging of bladder cancer.

### Example 25

### Unphosphorylated uTF-STP and uTF-STP-Pser258 Levels are Correlated with Grading of Tumors

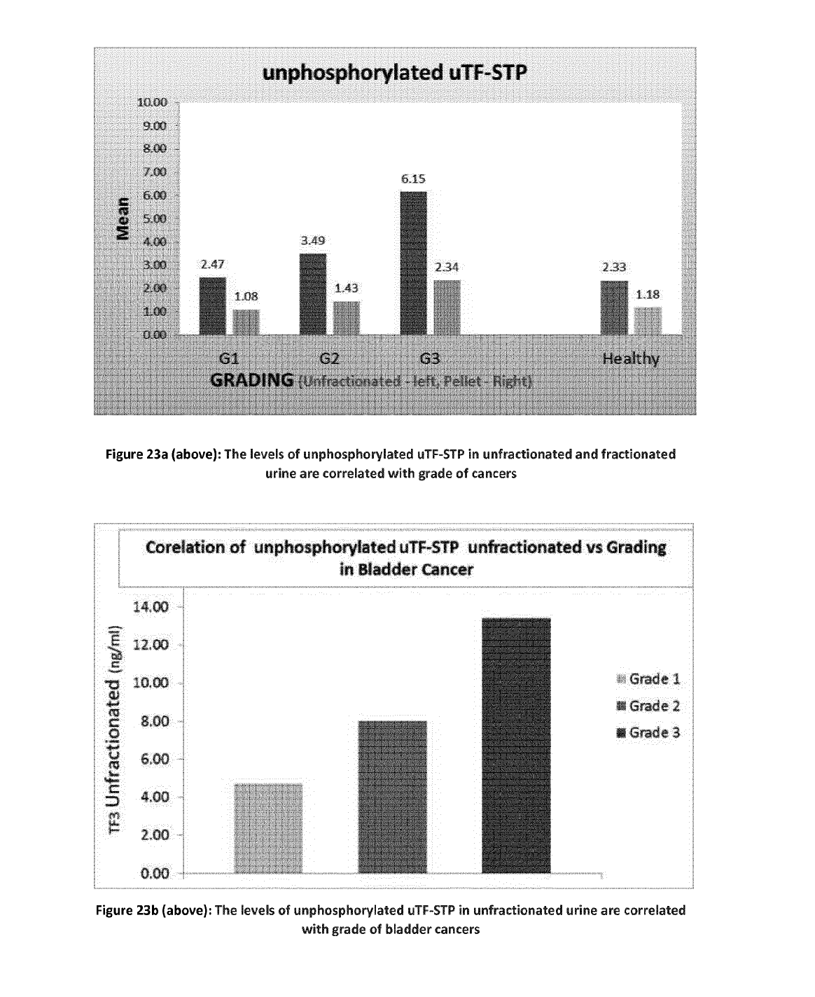

Grading is another important descriptive terms used by pathologists to characterize solid tumors. Grading is a qualitative indicator of the “aggressiveness” of tumors. We found clear correlation between tumor grade and level of unphosphorylated TF in different tumor types. The mean unphosphorylated TF levels measured in the unfractionated and the fractionated sample of all the tumor types combined steadily increase as the grade of the tumor increases from GI-G3 (FIG. 23a). Thus, unphosphorylated TF in the urine is a direct indicator of the grade of solid tumors.

Unphosphorylated uTF-STP levels in unfractionated samples are highly correlated with grading of bladder cancer. Low grade bladder cancers have low levels of unphosphorylated uTF-STP levels in unfractionated urine and high grade tumors have high levels of unphosphorylated TF-STP in unfractionated urine. Unphosphorylated uTF-STP levels in urine can be useful for determining the grade of a bladder cancer (FIG. 23b).

### Example 26

### Utility of Applying Algorithms to Population Screening

Combining the results of tests of the individual TF isoforms using the ELISA methods can be useful for screening populations as well as monitoring therapy and recurrence of tumors, in particular wherein said TF isoforms are found in urine (UTF isoforms). Applying an algorithm or applying sequential cut-offs for two or more of the TF isoforms can be useful for identifying the type of tumor that is detected by any one of the isoform tests. Individuals can be quantitated by ELISA for the presence of unphosphorylated TF-STP, TF-STP-Pser258 and TF-STP-Pser253 samples, and most preferably for the presence of unphosphorylated uTF-STP, uTF-STP-Pser258 and uTF-STP-Pser253 samples in unfractionated and fractionated urine samples. The ratios of the various biomarkers can also be calculated. Applying an algorithm or applying sequential cut-offs for each quantitative isoform result and/or each calculated ratio can be performed. The end results are samples that are highly likely to be from a patient with cancer and identify with high likelihood the particular type of cancer in the patient. In such a fashion one can screen undiagnosed populations and identify with high likelihood an individual with cancer. In some instances the algorithm will select for greater chance of identifying one tumor type over another tumor type.

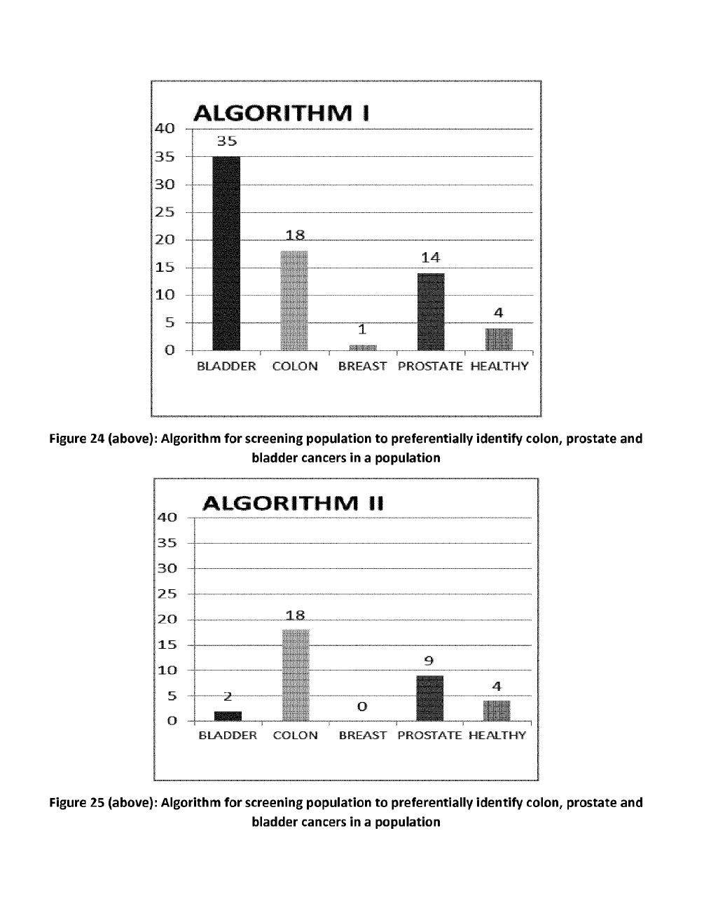

Algorithm to select bladder, colon and prostate cancers from population screened is as follows (FIG. 24). An algorithm is applied using the study population consisting of 45 healthy individuals and 167 patients in the following way. In this example, samples are selected if any two of the five criteria are met: 1) select all samples where the unphosphorylated TF levels in the unfractionated urines were >7.0 ng/ml; 2) uTF-STP-Per258 in unfractionated urine was >0.51 ng/ml; 3) uTF-STP-Pser258 in the fractionated urine was >0.41 ng/ml; 4) ratio of unfractionated uTF-STP to fractionated uTF-STP was >4.0; and 5) ratio of unfractionated uTF-STP-Pser253/fractionated uTF-STP-Pser253>49. The resulting population comprises 4 healthy individuals, 35 bladder cancer, 1 breast cancers, 18 colon cancers and 14 prostate cancers. In this analysis the sensitivity for detecting cancer is 68/167 (40.7%). The false positive rate was 4/45 (8%) and a positive predictive value 68/72 (94.4%).

An example of applying algorithms to preferentially select colon and prostate cancer from population screened is as follows (FIG. 25). An algorithm is applied using the study population consisting of 45 healthy individuals and 167 patients in the following way. Samples are selected if any two of the three criteria are met: 1) select all samples where the unfractionated uTF-STP-Pser253 >0.7 ng/ml; 2) ratio of unfractionated uTF-STP to fractionated uTF-STP was >4.0; and 5) ratio of unfractionated uTF-STP-Pser253/fractionated uTF-STP-Pser253>49. The resulting population comprises 4 healthy individuals, 2 bladder cancer, 0 breast cancers, 18 colon cancers and 9 prostate cancers. In this analysis the sensitivity for detecting cancer is 29/167 (17.3%). The false positive rate was 4/45 (8%) and a positive predictive value 29/33 (87.8%).

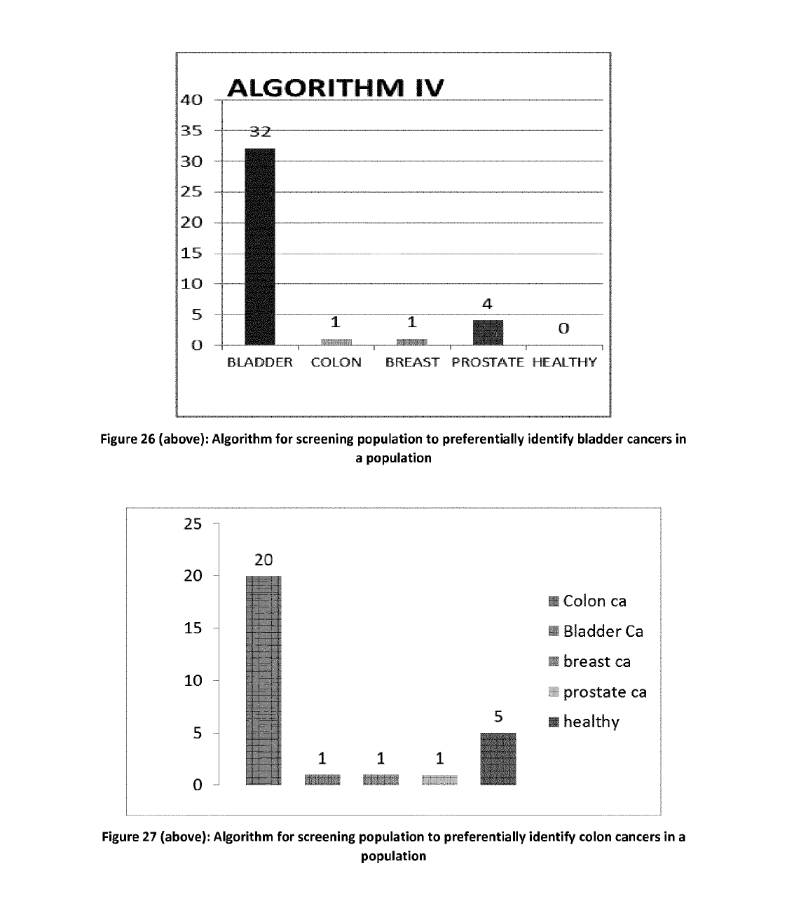

The algorithm in FIG. 26 is an example of applying algorithms to preferentially select bladder cancer from population screened is as follows. An algorithm is applied using the study population consisting of 45 healthy individuals and 167 patients in the following way. Samples are selected if two criteria are met: 1) select all samples where the unfractionated uTF-STP-Pser258>0.51 ng/ml; 2) fractionated uTF-STP-Pser258>0.46 ng/ml. The resulting population comprises 0 healthy individuals, 32 bladder cancers, 1 breast cancers, 1 colon cancers and 4 prostate cancers. In this analysis the sensitivity for detecting cancer is 38/167 (22.7%). The false positive rate was 0/45 (0%) and a positive predictive value 38/38 (100%).

The algorithm in FIG. 27 is an example of applying algorithms to preferentially select colon cancer from population screened is as follows. An algorithm is applied using the study population consisting of 45 healthy individuals and 167 patients in the following way. Samples are selected if three criteria are met: 1) select all samples where the unfractionated uTF-STP-Pser253 >0.7 ng/ml; 2) ratio of unfractionated uTF-STP-Pser253 to fractionated uTF-STP-Pser253 >49 and 3) uTF-STP-Pser258 <0.51 ng/ml. The resulting population comprises 5 healthy individuals, 1 bladder cancers, 1 breast cancers, 20 colon cancers and 1 prostate cancers. In this analysis the sensitivity for detecting cancer is 23/167 (13.7%) and for detecting colon cancer is 40.8% (20/49). The false positive rate was 5/45 (89.9%) and a positive predictive value for cancer 23/28 (82.1%).

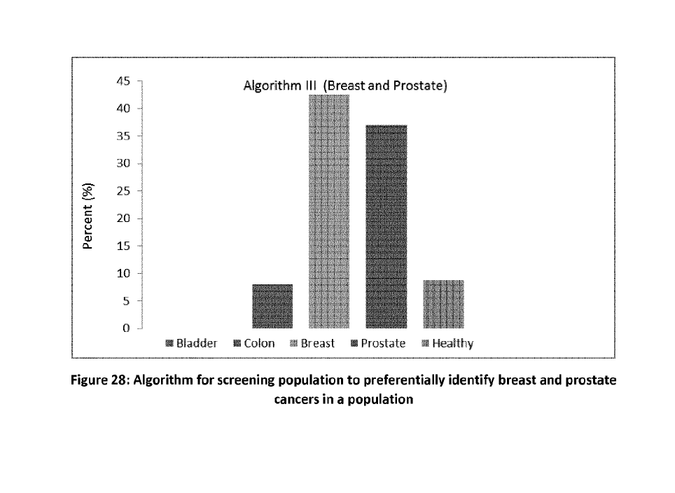

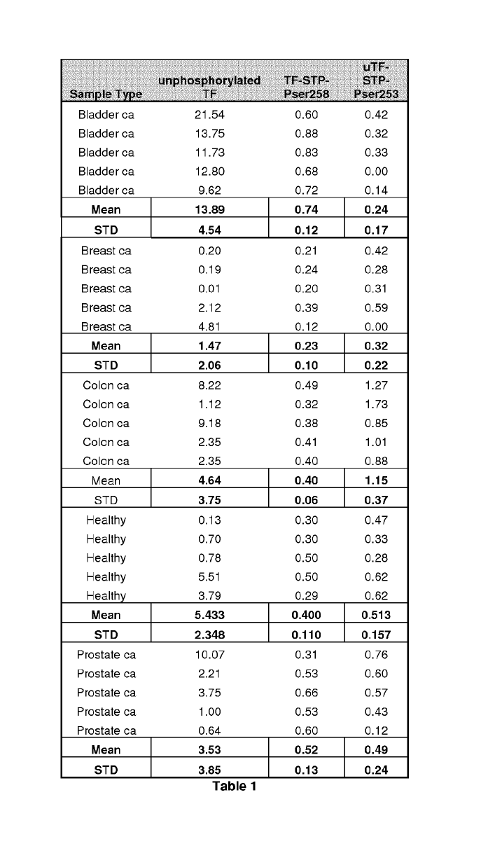

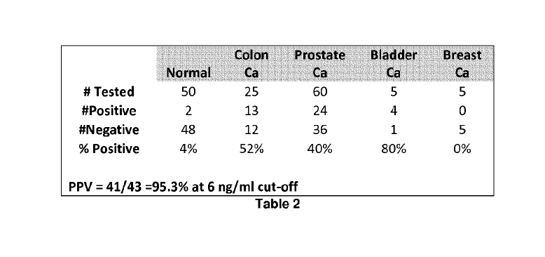

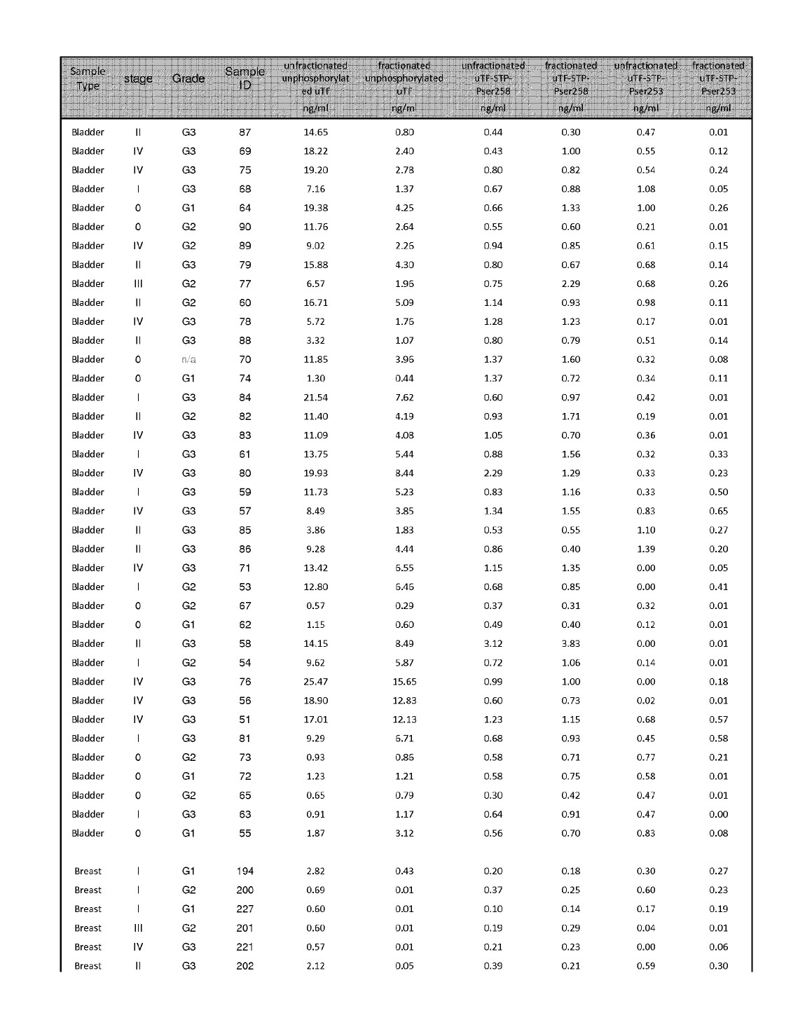

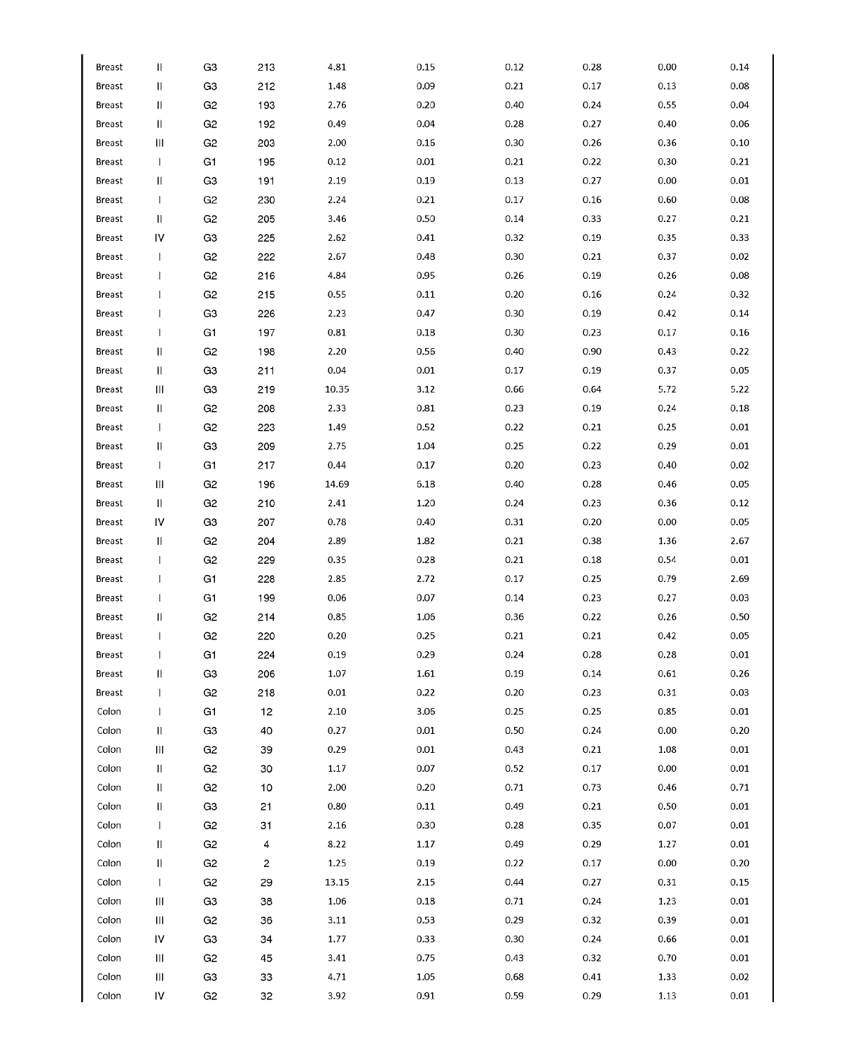

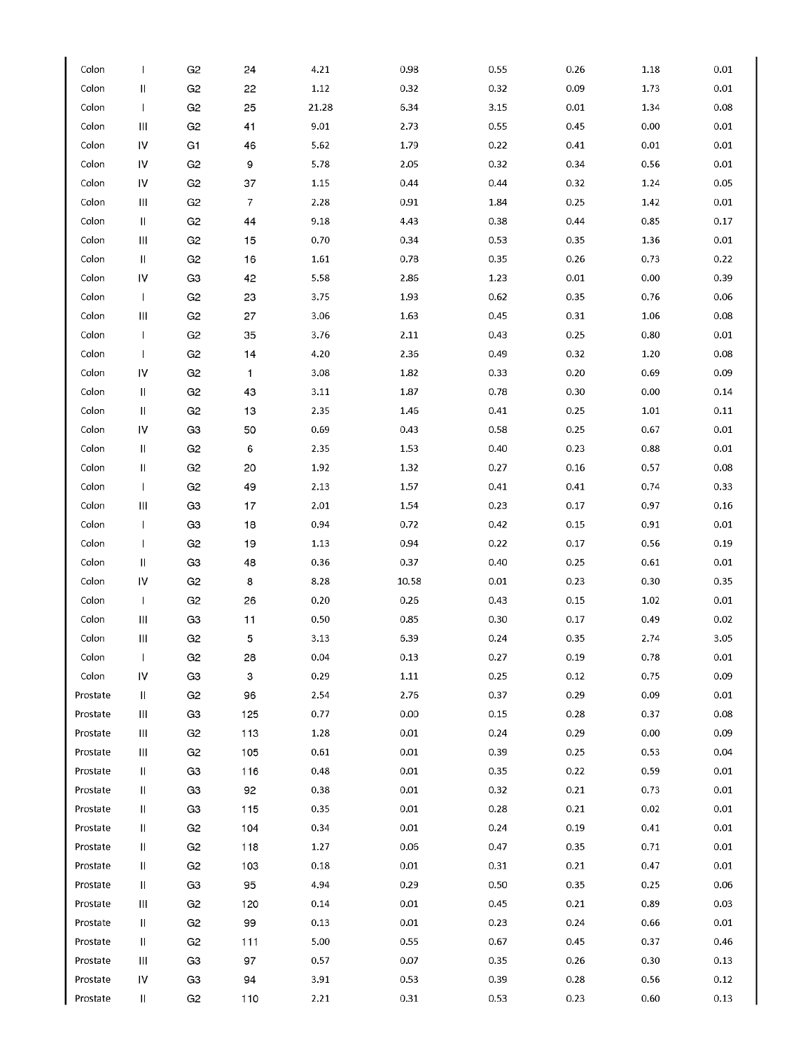

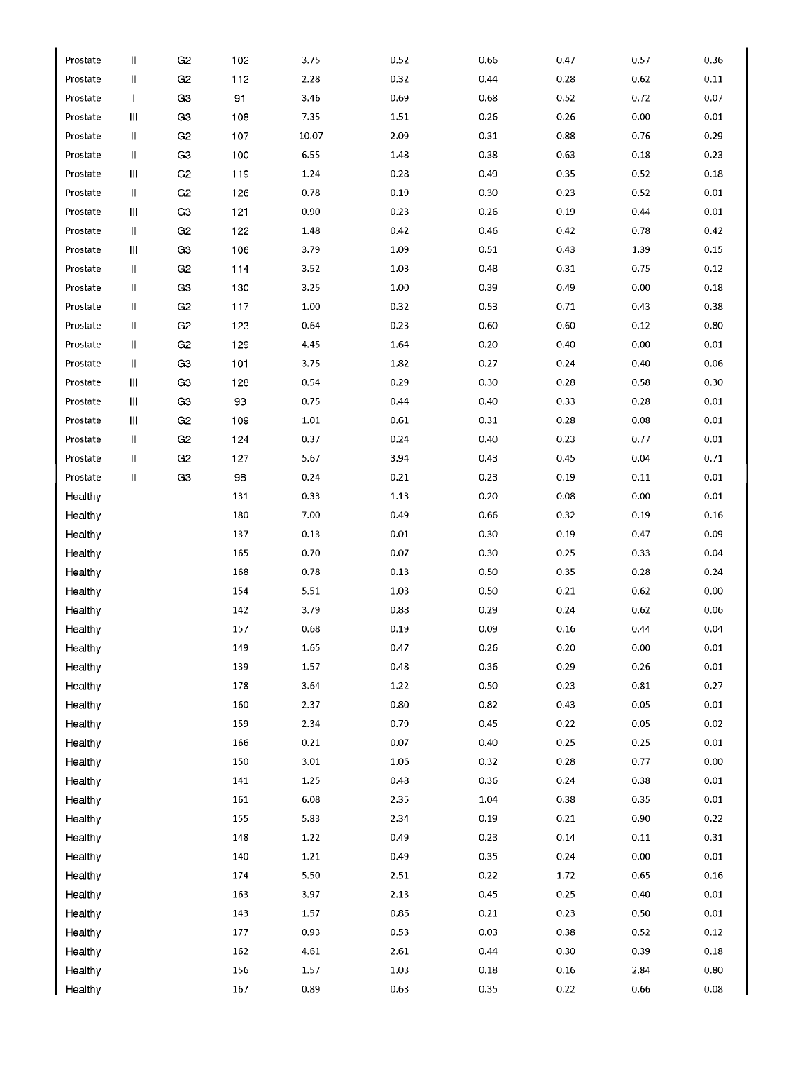

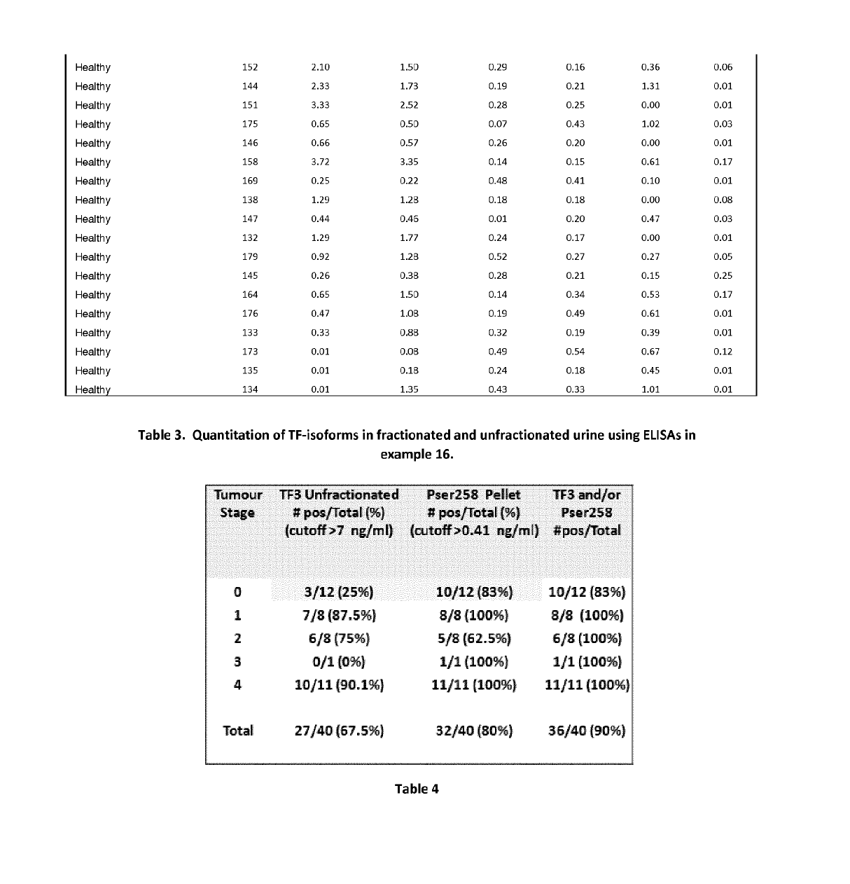

The algorithm in FIG. 28 is an example of applying algorithms to preferentially select prostate and breast cancers from population screened is as follows. An algorithm is applied using the study population consisting of 45 healthy individuals and 167 patients in the following way. Samples are selected if three criteria are met: 1) select all samples where the ratio of unphosphorylated uTF-STP in unfractionated and fractionated urine >4.0; 2) ratio of fractionated unphosphorylated uTF-STP to fractionated uTF-STP-Pser253 <3.6. The resulting population comprises 4/45 (8%) healthy individuals, 0% (0/38) bladder cancers, 17/40 (42%) breast cancers, 4/49 (8%) colon cancers and 15/40 (37.5%) prostate cancers. In this analysis the sensitivity for detecting cancer is 36/167 (21.5%) and for detecting breast cancer is 42%, prostate cancer 37.5% and colon cancer 8%. The false positive rate was 8% and a positive predictive value for cancer 36/40 (90%).

### REFERENCES

Adamson, 1992

Adamson 1993;

Ahamed J et al. PNAS 2006;103:13932;

Altschul et al., J. Mol. Bio1.215:403, 1990

Altschul et al., Nature Genet. 6:119, 1994,

Amirkhosravi et al., 1996;

Ausubel et al., Current Protocols in Molecular Biology eds 1995 (supplement)

Barany & Merrifield, The Peptides: Analysis, Synthesis, Biology. Vol. 2: Special Methods in Peptide Synthesis, Part A. pp. 3-284; 1979

Beaucage et al., Tetra. Lett. 22:1859-1862, 1981;

Bogdanov et al.,2003;

Bogdanov et al.,2006;

Brown et al., Meth. Enzymol. 68:109-151, 1979;

Buchner et al., Anal. Biochem. 205:263-270, 1992;

Bukovsky et al, 1992,

Caldwell et a1,1963

Carson and Yoder,Blood Coagul Fibrinolysis (1992) 3: 779-787

Carty et al, 1990

Chen V M et al. Biochem 2006;45:12020-8;

Collier and Ettelaie, J. Biol. Chem. (2011) 286:11977-11984; Dorfleutner and Ruf, Blood (2003) 102:3998-4005)

Colluci, 1991;

Corpet et al., Nucleic Acids Research 16:10881, 1988;

del Conde,

Edwards et al.

Gluzman, Eukaryotic Viral Vectors, Cold Spring Harbor Laboratory, ed., 1982

Guo et al., 2001

Higgins and Sharp, Gene 73:237, 1988;

Higgins and Sharp, CABIOS 5:151, 1989;

Huse et al., Science 246:1275, 1989

Joist et al, 1967

Kabat et al., Sequences of Proteins of Immunological Interest, U.S. Department of Health and Human Services, 1991

Koyama et al., 1994 (British J Haematology 87:343-347)

Kurosawa et al, 1984

Lwaleed 1996,

Lwaleed et al, 1997a

Lwaleed et al, 1997b

Lwaleed et al, 1999

Lwaleed et al, 1998

Lwaleed et al, 2007;

Matsuda et al, 1979;

Matsumura and von Kaulla, 1968;

Matsumura et al, 1968;

Matsumura et al, 1970,

Merrifield et al., J. Am. Chern. Soc. 85:2149-2156, 1963,

Napoleone, Di Santo & Lorenzet, 1997

Narang et al., Meth. Enzymol. 68:90-99, 1979;

Needham, VanDevanter et al., Nucl. Acids Res. 12:6159-6168, 1984;

Needleman and Wunsch, J. Mol. Bioi. 48:443, 1970;

Østerud, Olsen & Wilsgard, 1990;

Pearson and Lipman, Proc. Natl. Acad. Sci. U.S.A. 85:2444, 1988;

Pearson and Lipman, Proc. Natl. Acad. Sci. U.S.A. 85:2444, 1988.

Pendurthi et al Blood 2007;3900-8

Pluckthun, Biotechnology 9:545, 1991;

Qi H et al, 1996

Roth, 1994

Ryan et al., 1992

Ryden et al. (International Journal of Cancer 126:2330-2340, 2010)

Sambrook et al., Molecular Cloning: A Laboratory Manual, 2nd ed., vol. 1-3, ed. Cold Spring Harbor

Laboratory Press, Cold Spring Harbor, N.Y., 1989;

Sanchez-Nino et. al

Saxena et al., Biochemistry 9: 5015-5021, 1970,

Scopes, R., PROTEIN PURIFICATION, Springer-Verlag, N.Y., 1982

Shrimpton,

Smith and Waterman, Adv. Appl. Math. 2:482, 1981;

Spillert and Lazaro,

Stewart et al., Solid Phase Peptide Synthesis, 2nd ed., Pierce Chern. Co., Rockford, III., 1984.

Thiagarajan & Lopez, 2005

Uby, J., Immunology, 3rd Ed., W.H. Freeman & Co., New York, 1997

von Kaula et al, 1954

von Kaula et al, 1965

von Kaula et a1,1963

Ward et al., Nature 341:544,1989

Wiggins et al, 1987

Zacharaski et al, 1974

1.-50. (canceled)

51. A method of quantifying one or more tissue factor signal transduction peptide (TF-STP) isoforms in a test sample obtained from a subject, the method comprising:
(i) contacting and incubating the sample with a capture reagent immobilized to a solid support, wherein the capture reagent is a first polyclonal or monoclonal antibody that recognizes one antigen site on the TF-STP isoform;
(ii) separating the biological sample from the immobilized capture reagent-target TF-STP isoform complex;
(iii) contacting the immobilized capture reagent-target TF-STP isoform complex with a second detectable polyclonal or monoclonal antibody that binds directly or indirectly to a second antigen site on the captured TF-STP isoform; and
(iv) measuring the level of TF-STP isoform bound to the capture reagents using a detection means for the detectable antibody,
wherein said method is repeated using different antibodies in order to quantify one or more different TP isoforms in the same sample.

52. The method of claim 51 wherein said test sample is fractionated or unfractionated urine.

53. The method of claim 51 wherein said polyclonal antibody or monoclonal antibody comprises SEQ ID NO: 11 or SEQ ID NO: 12 or a functional fragment thereof.

54. The method of claim 53, wherein the antibody comprises (i) a heavy chain comprising SEQ ID NO: 11, or a functional fragment or variant thereof; and/or (ii) a light chain comprising the amino acid sequence as defined in SEQ ID NO: 12, or a functional fragment or variant thereof.

55. A method according to claim 51 wherein the one or more TF-STP isoform is selected from the group consisting of phosphorylated TF-STP and unphosphorylated TF-STP.

56. The method of claim 55, wherein the phosphorylated TF-STP isoform is selected from the group consisting of uTF-STP-Pser253, TF-STP-Pser253, TF-STP-Pser258, and uTF-STP-Pser258.

57. The method of claim 56, wherein the phosphorylated TF-STP isoform comprises one or more of SEQ ID NOs: 4, 5, 6, 7, 8 or 14.

58. A method according to claim 54 wherein the test sample is selected from the group consisting of fractionated or unfractionated urine, saliva, whole blood, plasma, platelet rich plasma (PRP), platelet poor plasma (PPP), and pooled normal plasma (PNP).

59. A method according to claim 54 wherein two or more TF-STP isoforms are quantified, further comprising the step of calculating the ratio of the two or more TF-STP isoforms and comparing the ratio of the two or more TF-STP isoforms in the test sample to the ratio of those isoforms in a normal or healthy control sample, wherein a ratio of the two or more TF-STP isoforms in the test sample which falls outside of a predetermined normal range is indicative of a characteristic of a disease.

60. A method according to claim 51 wherein two or more of the quantities and/or ratios falling outside of a predetermined normal range is indicative of a characteristic of a disease.

61. A method according to claim 60,wherein the disease is a cancer and the characteristic of the cancer is the cancer type, the presence of the cancer, the stage of an associated tumor, or the grade of an associated tumor.

62. A method according to claim 51 wherein at least one step, preferably all of the steps of said method is carried out in vitro.

63. A method according to claim 54 wherein the one or more TF-STP isoforms in a test sample is quantified by mass spectroscopy, HPLC, gas chromatography, or immunoassay.

64. A method according to claim 63 wherein the one or more TF-STP isoforms in a test sample is quantified by an Enzyme-Linked Immunosorbent Assay (ELISA).

65. A method according claim 51 wherein the one or more TF-STP isoforms are uTF-STP isoforms.

66. An antibody or functional fragment thereof comprising at least one of the following polypeptide sequences, or a polypeptide having at least 90% identity thereto:
SEQ ID NO: 11 or SEQ ID NO: 12.

67. A pharmaceutical composition comprising an antibody or functional fragment thereof as defined in claim 66, and a pharmaceutically acceptable carrier.


SEQUENCE LISTING (14 sequences)

SEQ ID NO: 1 - protein, 10 aa, Homo sapiens
CKENSPLNVS

SEQ ID NO: 2 - protein, 10 aa, Homo sapiens
CRKAGVGQSW

SEQ ID NO: 3 - protein, 19 aa, Homo sapiens
CRKAGVGQSWKENSPLNVS

SEQ ID NO: 4 - protein, 10 aa, Artificial Sequence
  Amino acids 255-263 of human TF with phosphorylation of serine
  MOD_RES (5)..(5): PHOSPHORYLATION
CKENSPLNVS

SEQ ID NO: 5 - protein, 11 aa, Artificial Sequence
  Amino acids 245-254 of human TF with phosphorylayed serine 253
  MOD_RES (9)..(9): PHOSPHORYLATION
CRKAGVGQPSW

SEQ ID NO: 6 - protein, 19 aa, Artificial Sequence
  Amino acids 245-263 of human TF with phosphorylated serine 253
  MOD_RES (9)..(9): PHOSPHORYLATION
CRKAGVGQSWKENSPLNVS

SEQ ID NO: 7 - protein, 19 aa, Artificial Sequence
  Amino acids 245-263 of human TF with phosphorylated serine 258
  MOD_RES (14)..(14): PHOSPHORYLATION
CRKAGVGQSWKENSPLNVS

SEQ ID NO: 8 - protein, 19 aa, Artificial Sequence
  Amino acids 245-263 of human TF with phosphorylated serines at 253 and 258
  MOD_RES (9)..(9): PHOSPHORYLATION
  MOD_RES (14)..(14): PHOSPHORYLATION
CRKAGVGQSWKENSPLNVS

SEQ ID NO: 9 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 10 - protein, 263 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 11 - protein, 148 aa, Artificial Sequence
  Amino acid sequence of heavy chain variable region of anti SEQ ID NO5 antobody
MRVLILLWLFTAFPGILSDVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARIRGYLAMDYWGQGTSVTVSSESQSFPNVFPLV

SEQ ID NO: 12 - protein, 167 aa, Artificial Sequence
  Amino acid sequence of light chain variable region of anti SEQ ID N5 antibody.
MRSPAQFLGLLVLWIPGSTADIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPPTFGGGTKLEIKRADAAPTVSIFPPSSEQLTSGGASVVCFLNNFYPK

SEQ ID NO: 13 - protein, 9 aa, Homo sapiens
KENSPLNVS

SEQ ID NO: 14 - protein, 9 aa, Artificial Sequence
  Amino acids 255-265 of human TF with phosphorylated serine 258
  MOD_RES (4)..(4): PHOSPHORYLATION
KENSPLNVS